SEQUENCE LISTING (12 sequences)

SEQ ID NO: 1 - DNA, 868 bp, Homo sapiens
  misc_feature (87)..(87): n is a, c, g, or t
  misc_feature (122)..(122): n is a, c, g, or t
  misc_feature (188)..(188): n is a, c, g, or t
  misc_feature (198)..(198): n is a, c, g, or t
  misc_feature (206)..(206): n is a, c, g, or t
  misc_feature (217)..(217): n is a, c, g, or t
  misc_feature (334)..(334): n is a, c, g, or t
  misc_feature (421)..(421): n is a, c, g, or t
  misc_feature (510)..(510): n is a, c, g, or t
  misc_feature (568)..(568): n is a, c, g, or t
  misc_feature (637)..(637): n is a, c, g, or t
  misc_feature (705)..(705): n is a, c, g, or t
cttgtatccagaatatataaagaacgcctacaactcaacaataaaaaaacgaatttcccaacaaaaaaacggacaaaggacacgaanagaccgtttacaaaagaagaaatggaaataactancgaacatgaaaaatgttcaacctcactaataatcaaagaaatgcaaattaaaacaacaatgagatnccgttcttcntcgtctancaaactggcanagatataaaaagataatakccagtgttggtgaggatgtggagaaacgggcactctcatacactgctggtgggagtataaattggtacaacctttctggaaggcaatttggcaatatntatcaaaagccttaaaaatgttcataccctttgacccagcaattccacttctaggaatctatcctaaggaaataatcagaaatgtgnacaaagatttacgtacaaagatgttcaccgcagtattatttataatagcaaaaaattggaaacaacctaaatgtccaataataggggantggttaaataaattatggtacatccatacaatggaatattatgcagccattaaaaatnatgttttcgaagaatatttaatgacatgggaaaatgctcatgatataatgttaagtgaaaaaagcaggntacaaaactgtatatacagtatgatctcaactttgttataaaattacatatataaatgtatacgtatntacatagaaaaaagactggaaggaaatacaccaaaatgttaacagtggttatctctgggtggtgggattatgggtgatttttattttcttttttctttgtattttctgtattttccaaattttctacaatgaacatgtattacttttataatcagaaaaaaaa

SEQ ID NO: 2 - DNA, 5024 bp, Homo sapiens
  misc_feature (41)..(41): n is a, c, g, or t
  misc_feature (48)..(48): n is a, c, g, or t
  misc_feature (65)..(65): n is a, c, g, or t
  misc_feature (250)..(250): n is a, c, g, or t
  misc_feature (325)..(325): n is a, c, g, or t
  misc_feature (337)..(337): n is a, c, g, or t
  misc_feature (383)..(383): n is a, c, g, or t
  misc_feature (705)..(705): n is a, c, g, or t
  misc_feature (1096)..(1097): n is a, c, g, or t
  misc_feature (1236)..(1236): n is a, c, g, or t
  misc_feature (1302)..(1302): n is a, c, g, or t
  misc_feature (1306)..(1306): n is a, c, g, or t
  misc_feature (1308)..(1308): n is a, c, g, or t
  misc_feature (1319)..(1319): n is a, c, g, or t
  misc_feature (1590)..(1590): n is a, c, g, or t
  misc_feature (1617)..(1617): n is a, c, g, or t
  misc_feature (1702)..(1702): n is a, c, g, or t
  misc_feature (2136)..(2138): n is a, c, g, or t
  misc_feature (2257)..(2257): n is a, c, g, or t
  misc_feature (2271)..(2271): n is a, c, g, or t
  misc_feature (2493)..(2494): n is a, c, g, or t
  misc_feature (2518)..(2518): n is a, c, g, or t
  misc_feature (2529)..(2529): n is a, c, g, or t
  misc_feature (2538)..(2538): n is a, c, g, or t
  misc_feature (2904)..(2904): n is a, c, g, or t
  misc_feature (2906)..(2908): n is a, c, g, or t
  misc_feature (2952)..(2953): n is a, c, g, or t
  misc_feature (3077)..(3077): n is a, c, g, or t
  misc_feature (3247)..(3247): n is a, c, g, or t
  misc_feature (3349)..(3349): n is a, c, g, or t
  misc_feature (3352)..(3352): n is a, c, g, or t
  misc_feature (3361)..(3361): n is a, c, g, or t
  misc_feature (3363)..(3363): n is a, c, g, or t
  misc_feature (3377)..(3377): n is a, c, g, or t
  misc_feature (3379)..(3381): n is a, c, g, or t
  misc_feature (3383)..(3383): n is a, c, g, or t
  misc_feature (3387)..(3387): n is a, c, g, or t
  misc_feature (3389)..(3389): n is a, c, g, or t
  misc_feature (3393)..(3393): n is a, c, g, or t
  misc_feature (3395)..(3395): n is a, c, g, or t
  misc_feature (3400)..(3400): n is a, c, g, or t
  misc_feature (3402)..(3402): n is a, c, g, or t
  misc_feature (3407)..(3407): n is a, c, g, or t
  misc_feature (3762)..(3762): n is a, c, g, or t
  misc_feature (3905)..(3906): n is a, c, g, or t
  misc_feature (3911)..(3911): n is a, c, g, or t
  misc_feature (4704)..(4704): n is a, c, g, or t
  misc_feature (4740)..(4740): n is a, c, g, or t
  misc_feature (4783)..(4783): n is a, c, g, or t
  misc_feature (4805)..(4805): n is a, c, g, or t
  misc_feature (4807)..(4807): n is a, c, g, or t
  misc_feature (4827)..(4827): n is a, c, g, or t
  misc_feature (4836)..(4836): n is a, c, g, or t
  misc_feature (4855)..(4855): n is a, c, g, or t
  misc_feature (4887)..(4887): n is a, c, g, or t
  misc_feature (4896)..(4896): n is a, c, g, or t
  misc_feature (4921)..(4921): n is a, c, g, or t
  misc_feature (4944)..(4944): n is a, c, g, or t
  misc_feature (4951)..(4951): n is a, c, g, or t
  misc_feature (4954)..(4954): n is a, c, g, or t
  misc_feature (4959)..(4959): n is a, c, g, or t
  misc_feature (4998)..(4998): n is a, c, g, or t
  misc_feature (5008)..(5008): n is a, c, g, or t
aaggagtttcacttctggaatggcagcatgaggagctccgnagacccnctccccagcgaaacaancataactggtgaaaattatttttaaaaaaacaaccatttaaagtctctggaaattgtcctaagggcatacagcaaatgaagaaacatttattcaagaaaatctactaaatctcagtaagaacagtgagagtctgtggcacttgagccacgacccgctcccaccctcccccctccccagctcagcntgacagaagctccactccgggcgggtgcggccaagaagacggggctccctctcccctcagctcccagtcaagggntacggtatctcnccgggaggggcaggccgccagcatttctcatcccctccagctccgngttgcagaggctaaattccaggtgagtgtagctgagaggtcgggggctcccttcctccacccagcccccactcatagggcggaggctctaccccaggcgcggcaggccgagaatactggggccctgattgccctcaccccagctcgctcatagggcggaggttccacgccgggagaggcaagccgagaagaccagaggctaccgcccccgcccagcgccctgctcataaagcaggggtgtcactccgagagaagcgggccactgtccccgcccccagctccggagcagtggctcagagattttgcccagggggagaggcagnccataagaacagagagctccgaagctctccccaaaggaactgactttatttgaaacagagtgtggggaagttcaagcctaagggtactctcgaaaacaatggagattttggtggtaagcaattaagaggaggctggtagctccatgagagcaacaagctaaaccataggccagctagtttaccagagagaaccagggaaagagacagctaagaagagccctcctggggtcagaacaaacctcaaagactggcctcaaaaactacccctrcaaaggggcccgaatttaattggatcagactgtggagcaatttatgccccagggcattgtcgaaaacaatagagcaatcagccggcaattagtggagcctaacagctgggtgtgataccaanngaggcagacagcttaacagagagatcagggaaagagacagtcaaagagagccctgctaaaaccactgtcatcccagggtgactgtgcgcatgcccaaggctgcgccctctgaggagcgacatcagaggcttcacactgngggggaaatagacttcactaaaatagtccagccaagtcactaaacaaataaacaagcaaaaacaancacnangagccgggggnggggaatcagtatccagagttgctacaatatattacctaaaatgtccagttttcaacaaaaaattatgagacatgcaaagaaacaggaaagtgtgacccatacacaggaaaaaaagcaggcaacagaaactgcctgtgagagggcccagatgtcggatttagcagacaaagacttcaaagcagccattataaatatgttcaaagaactaaaggaaaccatgcttaaagaagtaaaggaaggtatgatgacaatgtctcatcaaatagagantatcaataaagagatagaaattataanaaaaaaccaaatggaaattctggagttgaaaagtacaataactgaaatgaaaaattcactagaggggctcaacagtagatttganctggcagaagaaaagaatcagtraacttgaagatagatcaatagagattatgcaatctgaagaacagaaagaaaaaaaagaatgaagaaaaatgaacagagcctcagagaaatgtgggacaccatyaagcataccaacatatacatacatggacagacaaacaacatatacataatgggagtaccagaaggagaggagaagagagagaaaggagcagaaaaaatatttgaagaaataatggctaaaaacttcccaaatttgatgaaaaacattaatattaatctacacatccaagaagctcaataaactccaagtaggataaactcaaagagatccacacctagacacatcatagtcaaaatgttgaaagacaaagacaaagagaaaatcttgaaagcagcaagagaaaaatgactcatcacatacaagggaannnacctcaataagattaacagctgacttctcatcagaaacaatggaggccagaaggcagtgggatgacatattcaaagtgctgaaagaaaaaaaaaaaaaaaaaaaaacaaaaaaahacanaaacaaatacaacnytacctgtcaaccaagaattctatatccagcaaaactatctttcaaaaatgaaggtgaaataaagacattcccagataaacaaaaactgagagaatttgttgctagcagacctaccttacaagaaatactaaaggaagagttcttcaggctgaaaggcaagtgacaccagatagtaattcaaatccacataaaaaaataaagagacacacactaagtaaagnncactagtaaaggtaattatgtagnaagacagtaanttaattatnaaagrcatgtakgtaattataaaagacagtataaatgcatatttcttctttcttctcttaactgatttaaaaagcaattgtataaaacaatatgtatataattgtattgttgggcctataacatatagaaatgtaatatatttgacaataacagcacaaaggaggtgggtgggagcaaagctgtattggagtaaggaaatgacaccagatggtaacttgaatccacaggaacaaatgaagagaaccagaaatggtaaataagaaggttaatataacaaactctataaatatatacttgttctcctttcttctcttctttaaaagacataaaattatataaagtaataattataacaaatgtatttntnnnataataatgttgggtttgtaacatatatagatgtatatatattnntattgtaatatgtataacaataatagcacaaaaaaggagaaaaaggaatagagctatataggagtaacatttctatatctcactggaattaagttagtataaatctgaagtagattctgataangttaagatgtatatggtaagccctagagcaaccactaagaaaataacttaaaaaaatatagtaaaaaaaatcattaaagaaattaaaatgttacactagaaaatattcacttaatgcaaaagaaagcagtaaaggaggaatagaggaacaaaaaagacatgagacatatnacatatagaaaacaaaaagtaaaatggcagatataaatccaactatatcaatataacattaaatgtgattatggattaaryaaaatggcaraagctgtcagnctngagatttantntatataaatccaantnnntngttnanatgntnagacngntaatncaaatatcaataataacattaaatgtgaatggattaaacaatccaatcaaaaggcagagattgtcagactggataaaaaaaaaaaaacaagatccaactatatgctgtctacaggagacacactttagattcaaagatacaaatagrttgaaagtaaaaggatggaaaaagatatatcatgcaaacagcaaccataagaaagctggagtggctatactaatatcagacaaaatagactttaaaacaaaaaatgttactagagataaagagggacattttattatataatgataaaagggtcaaaagggtcaatccatcaggaagatataacaattataaacatatatgcatatanatatatgcacctaacaacagagcccccaaaatacatgaagcaaaaactgacagaaatgaagggagaaatagacaattcaacaataatagttggagacttcaataycccactttcaataatggatagaacaactaggcagaagnnaatangatcaacaaggaaatagaagacttgaacaacactataaaccaactagacctaacagacatctatagaacatttatagaacactcyatccaacaacagcagaatatacattcttctcaagtgcacatggaacattctccaggatagaccatatgctaggccataaaacaagyctcaataaatttatttaaaggattgaaataatacaaagtatgttctctgaccacaatggaatgaaattagaaatcaataacaaaaaatttgggaaatttacaaatatgtggaaattaaacaacacactcctaaataaccaatgggtcaaagaagaaatcacaagagaaattagaaaatactttgagatgaatgaaaatgaagacacaacataccaaaatttatgggatgcagctaaagcagtgyttagaggaaaatttatagctgtaaatgcctatattaaaaaagaagaaagatctcaaatcaataacctaaccttctaccttaagacactaaaaaaagaagagcaaactaaacctaaagcaagcagaaggaaggaaataataaagattagagcagaaattaatgaaatagaagaaaaacaatagagaaaatcaatgaaaccaaaagctggttctttgaaaagatcaacaaaattgacaaacctttagctagactgaccaagaaaaagagaagactcaaattactaaaatcagaaatgaaagagggaacattactactaaccttacagaaataaaaaggattataaaggaatactatgaacaattgtatgccaataaattnagataacttagatgaaatggacaaattcctagaaanyaagacacacaaactacyaaaactgactcaagaagaaataganaatctgaatagacctataaaantnaagagattgaattagtaatntaaaaactnccyacaaaaaaagcccagncccagatggcttcactggtgaattctccaaanatttaaaanagaattaataccaattattcacctnttccaaaaaatagaagaggaggnaayactnccnaactnattctatgaggccagtattatcctgataccaaaaccagncaaagacatnacaaaagaaaagaaaa

SEQ ID NO: 3 - DNA, 2761 bp, Homo sapiens
  misc_feature (49)..(49): n is a, c, g, or t
  misc_feature (58)..(58): n is a, c, g, or t
  misc_feature (63)..(64): n is a, c, g, or t
  misc_feature (73)..(73): n is a, c, g, or t
  misc_feature (98)..(98): n is a, c, g, or t
  misc_feature (105)..(105): n is a, c, g, or t
  misc_feature (141)..(141): n is a, c, g, or t
  misc_feature (148)..(148): n is a, c, g, or t
  misc_feature (202)..(202): n is a, c, g, or t
  misc_feature (237)..(238): n is a, c, g, or t
  misc_feature (259)..(259): n is a, c, g, or t
  misc_feature (263)..(263): n is a, c, g, or t
  misc_feature (266)..(266): n is a, c, g, or t
  misc_feature (308)..(308): n is a, c, g, or t
  misc_feature (319)..(319): n is a, c, g, or t
  misc_feature (331)..(331): n is a, c, g, or t
  misc_feature (348)..(348): n is a, c, g, or t
  misc_feature (352)..(352): n is a, c, g, or t
  misc_feature (397)..(397): n is a, c, g, or t
  misc_feature (415)..(415): n is a, c, g, or t
  misc_feature (452)..(452): n is a, c, g, or t
  misc_feature (458)..(459): n is a, c, g, or t
  misc_feature (464)..(464): n is a, c, g, or t
  misc_feature (471)..(471): n is a, c, g, or t
  misc_feature (485)..(485): n is a, c, g, or t
  misc_feature (510)..(510): n is a, c, g, or t
  misc_feature (526)..(526): n is a, c, g, or t
  misc_feature (549)..(549): n is a, c, g, or t
  misc_feature (594)..(594): n is a, c, g, or t
  misc_feature (608)..(609): n is a, c, g, or t
  misc_feature (611)..(611): n is a, c, g, or t
  misc_feature (614)..(614): n is a, c, g, or t
  misc_feature (627)..(627): n is a, c, g, or t
  misc_feature (629)..(629): n is a, c, g, or t
  misc_feature (649)..(649): n is a, c, g, or t
  misc_feature (740)..(740): n is a, c, g, or t
  misc_feature (745)..(745): n is a, c, g, or t
  misc_feature (774)..(775): n is a, c, g, or t
  misc_feature (861)..(861): n is a, c, g, or t
  misc_feature (900)..(900): n is a, c, g, or t
  misc_feature (923)..(923): n is a, c, g, or t
  misc_feature (1003)..(1003): n is a, c, g, or t
  misc_feature (1084)..(1084): n is a, c, g, or t
  misc_feature (1128)..(1128): n is a, c, g, or t
  misc_feature (1198)..(1198): n is a, c, g, or t
  misc_feature (1331)..(1331): n is a, c, g, or t
  misc_feature (1382)..(1382): n is a, c, g, or t
  misc_feature (1964)..(1964): n is a, c, g, or t
  misc_feature (2390)..(2390): n is a, c, g, or t
  misc_feature (2495)..(2495): n is a, c, g, or t
  misc_feature (2565)..(2565): n is a, c, g, or t
  misc_feature (2623)..(2623): n is a, c, g, or t
  misc_feature (2625)..(2625): n is a, c, g, or t
ctaatatacctaatatacaaaaaactcttaaaattgaaggataaaaagncaaaaaccnaatannaaaatgggnaaaagacatgaacagacaattcacnaaaaatnataaaatggcccttaagcatataaaaagatgttcancctcacntataattagagaaacgcaaattaaaactacaccgagataccatttctcacccancagatcggcaaaaattaaaaagtatggcaatatannctgttggcgaggctgtggggnaacnggnactctcatacactgctggtgggagtgcaaattggtacaactnctttggaaganaatttggcagtntctaataaaactacacntgcntttacactttgacccattagtcccacttctagaaatttaccctanagaaatacttctaacagntcaaaaatacacatgtacagggatgttcatagcagtnttattnntaatngtaaaanattggaaacaatcnaaatgtccatcagcaggagaatggntgaataaactatggtncatccacacaatggaatactatncagctgtaaaaaagaatgaggaagatctctgtaataatgtggagngatttcggaacatnntnttnagttgaaaaagcnangcgcaaaagagtatatatantatgctacccttcatataagaaagaaggggatatgagaaaatatacatatatctgctcatttgtgcaaaaagaaacacagaaaagataaancaganactaatgagattggttacccacagggaannggtgggaatggggaggaaaggacggaaggaatggggggcagtgacacttttctgagtatacctttttgtatagttctaacttttgnaaccatgttaatgtttcacatactcaagaaatgaataantaaaatcaacaaggatggggganaactcaaaatgaaatacaaacagaaacaaatgaaccwaactgtatttcaaatgaataacataaccacactgaagggggtnaggaagaaaagaactaacccaagtaacttttgaacacagtattttgactatatgccctcaggctaaagacaaaaagaactntaaacaaatattgaactctagttagtaggcttattttccgcagnggcatgggttagcaattctgaaactactttctgtatattctaggactgagcaaataagtaaatatattgnggataatgggagccaggtttctcactgtcggagaagggagttacaaatatggaaagggggaagactagaatgaaccctgtggtgttggattggaattggaggtatcagtgtgaactcatggtttttaatatanatagatatacagacagacagatatagaaatagatatagatatatatgtgtntgtgtatatgtgtatgtatatacgtacatatatttcctagctctgtccactgagagggcctagaagcaatgacaccccagtagcaatgagcacacctagcgcccagatcttggtttctaaataccattctccactaaaaggaaccagggctccttggagaaatggctgattccagggctggggcagggaaagtacaagatgagcctggaacatcttgttgtgccagaaagtaaggaagtgctcaaagaatgatggggacatgtcaaaaggacacaggagccagcttgaaggggctcccactggccaaatctgggacaatttgagcatcaaaataaataatgatagtaatggattataacccattgaataaaataagaatccatgagtccatactgatataaataaataaataaataaatgggggagaagggaaagctcttccttacagtagaatgccaactaataaatgtagaaggaatgatggaattagaaaatcaccatttggcaaccatcatagtaataattgattcaggcaagaatcatcaatggatgctaaaactagtgggtgaaagtttgatgagnaacaggatatttacatagtctcaaagtatctccccacaaaatacttattaattacaaaggggaaaatagtaactttacagtggagaaacctggcagacaccaccttaaccaagtgatcaaagttaacatcaccagtaatgggacaaatcgacatcatgtgcctcctgatatgatgcactgagaaggacacaacatcacttctgtggtattcctgccaaaaatgcataacctgaatctaatcatgaggaaacatcagacaaacccaaattgagggacattctacaaaataactggcctgtactcttcaaaaatgtcaaggtcatgaaagacaaagaaagactgaggaactgttccagattaaaggagactaaagagacatgacaactaaatgcaacgcgtgatcctggattggatcctggaccaganttttttttgctataaaggacattattgggacaactggcgaaatttgaataaggtctgtagattagataatagtattgtatcaatgttaatttcctgattttgatnattgtactgtggttatgtaagagaatgtccttgtttttaggaaatacacactgaagtatttaggggtaanggggcatcatgtctgcaacttactctcaaatggttcagaaaaaaaaatatgtatatgnanacagagaatgataaagcaaatgtggcaaaatgttaacatttggggaatctgggtgaagggtatacgggaattctttgtactattcttgcaacttttctgtaagtctgaaattatttcaaaataaaaagttaaaaaa

SEQ ID NO: 4 - DNA, 21 bp, Homo sapiens
gcactaaatgcctacaagaga

SEQ ID NO: 5 - DNA, 21 bp, Homo sapiens
gatagaccgctagcaagacta

SEQ ID NO: 6 - DNA, 21 bp, Homo sapiens
gaagttgaatctctgaataga

SEQ ID NO: 7 - DNA, 21 bp, Homo sapiens
ggacctcttcaaggagaacta

SEQ ID NO: 8 - DNA, 21 bp, Homo sapiens
ggagaggatgcggagaaatag

SEQ ID NO: 9 - DNA, 23 bp, Homo sapiens
actgatcgtgagaggcttcgtgg

SEQ ID NO: 10 - DNA, 23 bp, Homo sapiens
ggtcatttagggtgacaggcagg

SEQ ID NO: 11 - DNA, 23 bp, Homo sapiens
gctgtcatgggaatcacgaaggg

SEQ ID NO: 12 - DNA, 23 bp, Homo sapiens
aaggaagaccacttttaaggagg

(19) United States
(12) Patent Application Publication
(10) Pub. No.: US 2023/0272399 A1
(43) Pub. Date: Aug. 31, 2023

(54) INHIBITORS OF LINE1 AND USES THEREOF

(71) Applicant: T-ONE THERAPEUTICS S.R.L., Milano (IT)

(72) Inventors: Beatrice Bodega, Milano (IT); Federica Marasca, Milano (IT)

(21) Appl. No.: 18/016,526

(22) PCT Filed: Jul. 19, 2021
(86) PCT No.: PCT/EP2021/070181

§ 371 Date: Jan. 17, 2023

(30) Foreign Application Priority Data
Jul. 17, 2020 (EP) 20186492.3

(51) Int. Cl. C12N 15/113; A61K 45/06

(52) U.S. Cl. CPC C12N 15/1131; A61K 45/06; C12N 2310/11

(57) ABSTRACT

The present invention relates to a suppressor or inhibitor of (long interspersed element 1) LINE1 (L1) expression for medical use.

### FIELD OF THE INVENTION

The present invention relates to a suppressor or inhibitor of (long interspersed element 1) LINE1 (L1) expression for medical use, particularly for use in the treatment and/or prevention of primary or secondary immunodeficiency, or of pathologies that display an immunosuppressed phenotype, preferably of cancers and/or metastasis, more preferably of lung cancer, even more preferably non-small cells lung carcinoma (NSCLC), or colorectal cancer (CRC), or of viral diseases.

### BACKGROUND TO THE INVENTION

Transposable Elements (TEs) Account for Genome Evolution and Inter-Individual Genetic Variability.

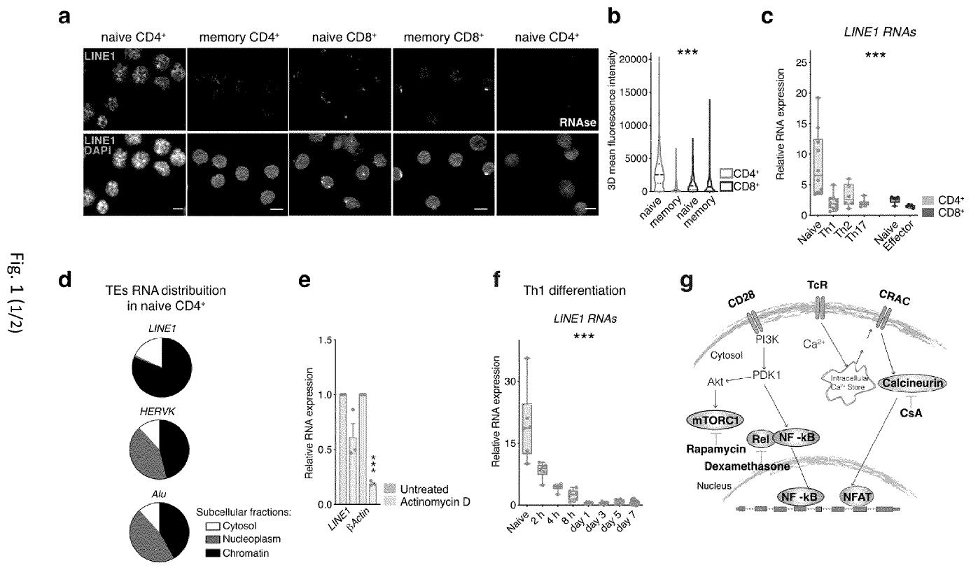

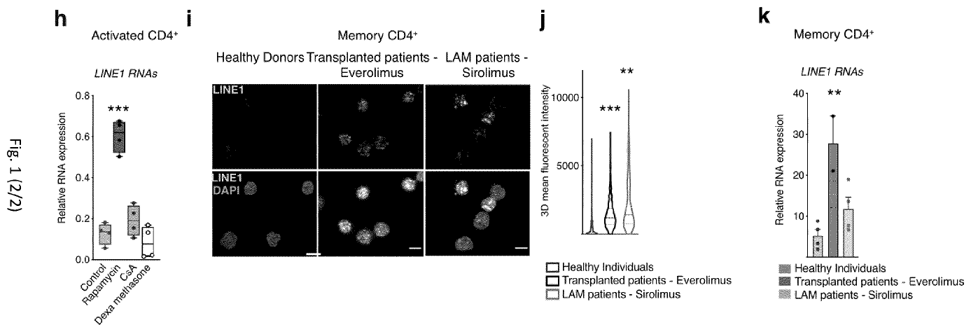

Two thirds of the human genome are constituted of repetitive elements (66%), among which Transposable Elements (TEs) accounts alone for the 40-45% of human genome composition^{1,2}. One fascinating question for genome biologists is to untangle the functions of this “dark side” of the genome, that still represents an “alive matter” on which evolution can play to generate novel functions. It is clear nowadays that TEs capability of regulating the genome resides mainly in generating a sophisticated plethora of RNA regulatory networks, which in turn influence the transcriptional output of the cell³⁻⁵. TEs are organized in four different classes and, with the exception of DNA Transposons, are mainly retrotransposons, which have acquired the ability by using RNA as intermediate to move via a ‘copy and paste’ mechanism. Retrotransposons include long interspersed elements (LINEs), short interspersed elements (SINEs) and long terminal repeats (LTR) retrotransposons. They are further classified as autonomous or non-autonomous depending on whether they have ORFs that encode for the machinery required for the retrotransposition⁶. LINE is a class of transposons very ancient and evolutionary successful. Three LINE superfamilies are found in the human genome: LINE1, LINE2 and LINE3, of which only LINE1 is still active. Full-length LINE1 (L1) elements are approximately 6 kb long and constitute an autonomous component of the genome. A LINE1 element has an internal polymerase II promoter and encodes for two open reading frames, ORF1 and ORF2 (FIG. 1)⁷. Once the L1 RNA is transcribed, it is exported to cytoplasm for translation, and subsequently assembled with the chaperone RNA-binding proteins ORF1 and the endonuclease and reverse transcriptase ORF2. These ribonucleoparticles are then reimported into the nucleus, where ORF2 makes a single-stranded nick and primes reverse transcription from the 3′ end of the L1 RNA. Reverse transcription frequently results in many truncated, nonfunctional insertions and for this reason most of the LINE-derived repeats are short, with an average size around 900-1000 bp. The LIs are estimated to be present in more than 500,000 copies in the human genome.

The L1 machinery is also responsible for the retrotransposition of the SINEs (which can be classified into three superfamilies: Alu, MIR, MIR3), non-autonomous retroelements without any coding potential, short in length (around 300 bp) and transcribed from polymerase III promoter (FIG. 1). The most represented human specific SINE superfamily, the Alu, is represented in 1,090,000 copies in the human genome⁸.

The LTR retrotransposons are initiated and terminated by long terminal direct repeats embedded by transcriptional regulatory elements. The autonomous LTR retrotransposons contain gag and pol genes, which encode a reverse transcriptase, integrase, protease and RNAse H (FIG. 1). Four superfamilies of LTR exist: ERV—class I, ERV(K) class II, ERV(L) class III, and MalR. MalR is the most represented superfamily of LTR, present in 240,000 copies⁹.

Evolutionary biologists hypothesize that self-replicating RNA genomes were the basis of early life on earth, and that the advent of reverse transcription had a pivotal function in the evolution of the first DNA genomes, the more stable deoxyribose-based polymers^{6,10}. From this perspective, multiple rounds of reverse transcription could have helped to expand both the size and the complexity of the human genome. It is particularly evident in both mammals and plants that retrotransposons have massively accumulated, driving genome evolution. It is reported that L1 and Alu represent the most prominent catalysts of the human genome evolution¹¹ and that homologous recombination between TEs could have driven/drives mutations, chromosome rearrangement, deletions, inversions and translocations¹². TEs are a major source of somatic genomic diversity and interindividual variability¹³ and TEs insertions have been documented as physiologically occurring¹⁴⁻¹⁶. In particular L1 retrotransposition has been extensively described to take place in neurons, from fly to man¹⁷⁻¹⁹ a mechanism that is fine-tuned and epigenetically regulated in neural progenitor development and differentiation, contributing to the somatic diversification of neurons in the brain^{13,20} The deregulation of TEs activity is nowadays emerging as an important contributor to many different diseases, as it occurs in neurological, inflammatory and cancer diseases²¹⁻²³.

The hosts have developed many systems to control TEs expression and expansion²⁴ (thus, epigenetic modification and noncoding RNAs such Piwi interacting-RNAs) to contain the possible detrimental effects of their retrotransposition. This expansion has achieved a balance between detrimental and beneficial effects, possibly becoming a novel regulatory mechanism to promote genomic functions acquired through evolution³. It is nowadays accepted, both in mouse and in human, that TEs have been co-opted into multiple regulatory functions for the accommodation of the host genomes metabolisms and transcription, mediated both by their DNA elements and by their transcribed RNAs counterparts.

Not Just Transposition: TEs RNAs are a Prolific Source for Novel Regulatory Functions.

TEs were first discovered in maize by Barbara McClintock almost 80 years ago. She suggested these elements as “controlling elements” able to regulate the genes activity^{25,26}. Her theories, even if dismissed for a long time, were pioneering and with the advent of Next Generation Sequencing (NGS) technologies have been thoroughly revised. Currently emerging concept is that TEs interact with the transcriptional regulatory functions of the hosts genomes^{3,4,27,28}.

Although a massive portion of the literature has been centered on the study of the retrotransposition and the effects of the de novo insertions, it is worth to notice that TEs can have RNA regulatory functions decoupled from their retrotransposition.

International decade long projects as ENCODE (Encyclopedia of DNA Elements) and FANTOM (Functional Annotation of the Mammalian Genome) have produced and bioinformatically analyzed a vast number of datasets opening the way for studying TEs. These results revealed that TEs have precise functions in establishing and influencing the cell type specific transcriptional programs, creating regulatory networks that are fostered both by their genomic elements and the derived transcripts^{3,28}, revealing that the RNAs transcribed from this elements could have a myriad of functions, definitely changing the way in which many genomic concepts were written in textbooks²⁹.

These studies clarified that TEs can create novel or alternative promoters³⁰, promote the assembly of transcription factors³¹ and epigenetic modifiers and favor their spreading³² and the regulation of gene expression. Further, TEs in particular SINEs and HERVs, have been demonstrated to have function in 3D genome folding, as the binding sites for chromatin organizers³³⁻³⁵.

In the 2009 Faulkner et al.³⁶, demonstrated for the first time that TEs are widely expressed in human and mouse cell types with tissue-specific patterns of expression, suggesting a specific spatiotemporal activation of retrotransposons. Faulkner et al. further demonstrated that up to the 30% of the transcripts initiate within repetitive elements³⁶. It is interesting to notice that tissues of embryonic origin contain the highest proportion of transposable element-derived sequences in their transcriptomes, with specific expression of LTR in placenta and oocytes³⁷. In accordance, it was recently found that different classes of repeats are specifically enriched in genes with a definite spatiotemporal expression, further dictating their timing and magnitude of expression in development³⁸.

Within this scenario, TEs magnify the transcriptome complexity in different ways: generating antisense transcripts, usually in proximity to gene promoters³⁶; acting on the maturation of mRNAs via nursing alternative splicing sites for tissue specific exonization^{39,40} and providing alternative polyadenylation signals^{41,42} and sites for the RNA-mediated decoy⁴³. Furthermore, TEs contribute to RNA regulatory sequences within introns and untranslated regions (UTRs)³⁶. I is important to notice that TEs are major contributors to long noncoding RNAs (lncRNAs)⁴⁴⁻⁴⁵. In this scenario, an enhancer RNAs function was proposed for LTR derived transcripts, as required for pluripotency maintenance in mouse and human embryonic stem (ES) cells^{46,47}. Further, it has been demonstrated that LINEs and SINEs are expressed as RNAs tightly associated to the chromatin compartment, where they localized at euchromatin, suggesting a possible function of these RNAs in 3D genome folding⁴⁸. LIs have been described also as chromatin associated RNAs both in embryogenesis, regulating open chromatin accessibility^{49,50}, and in mouse ES cells, where they are involved in the regulation of genes required for cell identity maintenance and 2-cell stage differentiation⁵¹.

Although these seminal papers have increased the consciousness and the knowledge on functions of TEs, highlighting important epigenetic roles for transposons in embryogenesis and development, contribution of TEs to adult cell plasticity and diseases occurrence and progression is still poorly investigated. This as a result of the intrinsic difficulties in studying TEs, which due to their repetitive nature, high degree of homology, sequence divergence and degeneration render almost unfeasible the application of the technologies established for biallelic genes, in particular in bioinformatic.

Relevance of Studying T Cell Transcriptional Plasticity within Tumor Microenvironment

It is nowadays well demonstrated that innate and adaptive immune responses play a fundamental role in tumorigenesis; the interplay between tumor cells and immune system is defined as cancer immunoediting. Indeed, the most complex form of immunoediting is represented by the crosstalk between tumor infiltrating T lymphocytes (TILs) and tumor cells, that expose neo-antigens on their surface within the tumor microenvironment; this could result in either tumor elimination, equilibrium between immune response and residual tumor cell growth, or tumor escape from immune control⁵².

Tumor microenvironment can be very heterogenous in terms of the immune infiltrate abundance, composition and response⁵³; in particular, the relative abundance and effector functions of TILs can be inhibited by the development of a tumor specific transcriptional program able to disempower, exclude and evade the immune system⁵⁴. The tumor-dependent immunosuppressive mechanisms rely on a complex network that establishes within the tumor microenvironment and is based on the upregulation of modulatory molecules, collectively called immune checkpoints, whose function is only partially characterized⁵⁵. Nevertheless, these molecules (e.g. CTLA-4, PD-1, PDL-1) are target of immune checkpoint inhibitors (ICIs) therapy (immunotherapy), that unleashes the spontaneous anti-tumor immune responses in such a powerful way that it has created a paradigm shift in cancer therapy⁵⁶⁻⁵⁸. However, while it is quite clear that tumor types that are more antigenic because of the high mutational load (e.g., melanoma, lung, kidney, bladder) are more likely to respond to immunotherapy, it is less clear as to why most patients with these highly antigenic tumors do not have a durable response or do not respond at all to immunotherapy; indeed, the fraction of patients that do not respond remains high, and the efforts in the field are mainly focused on searching specific ICIs against novel surface markers expressed in T cells subsets, also defined at single cell level^{54,59-62}. Almost nothing is reported regarding the genomic and epigenetic mechanisms that govern the intratumoral dysfunctional state of TILs, with the aim to reestablish their function, acting on reversible mechanisms of transcriptional plasticity.

### BRIEF DESCRIPTION OF THE INVENTION

Inventors have characterize two most frequent types of human cancer where immunotherapy is more (non small cells lung carcinoma, NSCLC) or less (colorectal cancer, CRC) frequently used and effective. They are the first and second causes of death worldwide, respectively. Lung cancer is the most common cancer in terms of incidence (2.09 million cases estimated in 2018)⁶³, with NSCLC accounting for 84% of lung tumor cases⁶⁴, with an overall survival at five years up to 19%. Colorectal cancer (CRC) is the third most common cancer, accounting for 1.84 million estimated new cases in 2018, with a 60% overall survival at five years^{63,65}.

Inventors have found that these TEs containing transcripts represent novel therapeutic targets, unpredictable with another strategies, for promoting TILs transcriptional reshape leading to unleashed effector immune response.

It is therefore an object of the invention a suppressor or inhibitor of LINE1 (long interspersed element 1) (L1) expression for use in the treatment and/or prevention of primary or secondary immunodeficiency, or of pathologies that display an immunosuppressed phenotype, preferably of cancers and/or metastasis, more preferably of lung cancer, even more preferably non-small cells lung carcinoma (NSCLC), or colorectal cancer (CRC), or of viral diseases such as immunodeficiencies due to Human Immunodeficiency Virus (HIV) or Lymphocytic choriomeningitis virus (LCMV)

wherein L1 comprises or consists of a sequence having 100, 99, 98, 97, 96, 95, 90, 85, 80% of identity with SEQ ID NO: 1 and/or 2 and/or 3.

Preferably, L1 comprises or consists of a sequence having 100, 99, 98, 97, 96, 95, 90, 85, 80% of identity with SEQ ID NO: 1 or 2 or 3.

Preferably the L1 comprises or consists of SEQ ID NO:1, 2 and/or 3.

Preferably the L1 comprises or consists of SEQ ID NO:1, 2 or 3.

Another object of the invention is a suppressor or inhibitor of LINE1 (L1) expression for medical use wherein L1 comprises or consists of a sequence having 100, 99, 98, 97, 96, 95, 90, 85, 80% of identity with SEQ ID NO: 1 and/or 2 and/or 3.

Preferably, L1 comprises or consists of a sequence having 100, 99, 98, 97, 96, 95, 90, 85, 80% of identity with SEQ ID NO: 1 or 2 or 3.

Preferably the L1 comprises or consists of SEQ ID NO:1, 2 and/or 3.

Preferably the L1 comprises or consists of SEQ ID NO:1 or 2 or 3.

Preferably the suppressor or inhibitor is at least one molecule selected from the group consisting of:
a) a polynucleotide, such as antisense construct, antisense oligonucleotide, RNA interference construct or siRNA or a polynucleotide coding for it,
b) an antibody or a fragment thereof,
c) a polypeptide;
d) a small molecule;
e) a polynucleotide coding for said antibody or polypeptide or a functional derivative thereof,
f) a vector comprising or expressing the polynucleotide as defined in a) or e);
g) a CRISPR/Cas9 component, e.g. a sgRNA;
h) a host cell genetically engineered expressing said polypeptide or antibody or comprising the polynucleotide as defined in a) or e) or at least one component of g).

Preferably the polynucleotide is an isolated inhibitory nucleic acid targeting LINE1.

Preferably the inhibitory nucleic acid comprises a sequence of nucleotides that is complementary to 10 to 50 consecutive nucleotides of SEQ ID NO: 1, 2 or 3.

Preferably said inhibitory nucleic acid is at least one RNA inhibitor, preferably selected from the group consisting of: antisense oligo (ASO), gapmer, mixmer, shRNA, siRNA, stRNA, snRNA, sgRNA, more preferably said inhibitory nucleic acid is modified, such as 2′-deoxy-2′-fluoro-β-D-arabinonucleid acid (FANA) ASO, and/or comprises one or more modified bonds or bases.

Preferably, the ASO or FANA ASO comprises a sequence capable of hybridizing or complementary to a sequence comprising or consisting of: SEQ ID NO: 1, 2 or 3.

Preferably, the suppressor or inhibitor is used in T cells, more preferably CD4+ T naïve cells or a CD8+ T cell, Tumor infiltrating Lymphocytes TILs both CD4+ and CD8+, B cells, Natural Killer cells or Tumor cells.

Preferably the suppressor or inhibitor is used in combination with an immunotherapy and/or with a radiotherapy and/or chemotherapeutic agent and/or with targeted therapies which promote raising of new antigens and immunity response and/or with immunity system adjuvants, preferably said immunotherapy comprises administration of an immune checkpoint inhibitor and/or chimeric antigen receptor (CAR)-expressing immune effector cells, preferably the immune checkpoint inhibitor is an or comprises one or more anti-CD137 antibodies; anti-PD-1 (programmed cell death 1) antibodies; anti-PDLI (programmed cell death ligand 1) antibodies; anti-PDL2 antibodies; or anti-CTLA-4 antibodies.

Preferably the suppressor or inhibitor is used in Adoptive cell transfer (ACT), cell therapy treatment, mismatched bone marrow transplantation, mismatched NK cell infusion or cytokine-induced killer (CIK) cell infusion, or wherein said suppressor or inhibitor is injected in the tumour site, e.g. in intestine tumour, melanoma, or delivered by nanoparticles specifically to the site of interest.

Another object of the invention is a pharmaceutical composition comprising the suppressor or inhibitor as defined above and at least one pharmaceutically acceptable carrier, and optionally further comprising a therapeutic agent.

A further object of the invention is a method to modulate the commitment of naïve CD4+ T naïve cells towards any effector lineage and to modulate the effector response in dysfunctional T cells comprising the step of inhibiting LINE1 expression in said cells, wherein the step of inhibiting LINE1 expression in said cells is performed by means of at least one suppressor or inhibitor as defined above.

Another object of the invention is an isolated human T cell, B cell, NK cell or Tumor cell, wherein said cell is stably or transiently affected in the expression of LINE1 (L1), preferably said cell is a CD4+ T naïve cell or a CD8+ T cell, or a dysfunctional T cell, e.g. a TIL.

Preferably L1 comprises or consists of a sequence having 100, 99, 98, 97, 96, 95, 90, 85, 80% of identity with SEQ ID NO: 1 and/or 2 and/or 3.

Preferably said cell is a CD4+ T naïve cell or a CD8+ T cell, or a dysfunctional T cell, e.g. a TIL.

Preferably the L1 comprises or consists of SEQ ID NO:1, 2 or 3.

A further object is a composition comprising at least one cell or combinations thereof as defined above, said composition preferably further comprising at least one physiologically acceptable carrier.

The cell, or the composition as defined above may be for use as a medicament, preferably for use in the treatment and/or prevention of primary or secondary immunodeficiency, or of pathologies that display an immunosuppressed phenotype, preferably of cancers and/or metastasis, more preferably of lung cancer, even more preferably non-small cells lung carcinoma (NSCLC), or colorectal cancer (CRC), or of viral diseases such as immunodeficiencies due to HIV, Lymphocytic choriomeningitis virus (LCMV).

Preferably said cell or composition being used in Adoptive cell transfer (ACT), cell therapy treatment, mismatched bone marrow transplantation, mismatched NK cell infusion or cytokine-induced killer (CIK) cell infusion, or wherein said cell or composition is injected in the tumour site, e.g. in intestine tumour, melanoma, or delivered by nanoparticles specifically to the site of interest.

Preferably the ASO comprises or consists of a nucleic acid sequence that targets or is complementary to one of the following sequences (LINE1 ASOs):

LINE1-a
(SEQ ID NO: 4)
GCACTAAATGCCTACAAGAGA
LINE1-b
(SEQ ID NO: 5)
GATAGACCGCTAGCAAGACTA
LINE1-c
(SEQ ID NO: 6)
GAAGTTGAATCTCTGAATAGA
LINE1-d
(SEQ ID NO: 7)
GGACCTCTTCAAGGAGAACTA
LINE1-e
(SEQ ID NO: 8)
GGAGAGGATGCGGAGAAATAG

or the corresponding RNA sequence.

Preferably the sgRNA comprises or consists of a nucleic acid sequence that targets or is complementary to one sequence which is a unique, non coding portion flanking LINE1 element which is removed.

Preferably the sgRNA comprises or consists of a nucleic acid sequence that targets or is complementary or is at least 75, 80, 85, 90, 95, 96, 97, 98, 99, 100% identical to one of the following sequence:

IFNGR2-F
(SEQ ID NO: 9)
ACTGATCGTGAGAGGCTTCGTGG
IFNGR2-R
(SEQ ID NO: 10)
GGTCATTTAGGGTGACAGGCAGG
ARCP2-F
(SEQ ID NO: 11)
GCTGTCATGGGAATCACGAAGGG
ARCP2-R
(SEQ ID NO: 12)
AAGGAAGACCACTTTTAAGGAGG

or to the corresponding RNA sequence.

The SEQ ID Nos 1-3 are retrotransposition incompetent and are those that the inventors have discovered as specifically expressed in T-lymphocytes (naïve and dysfunctional). Therefore, inhibiting the expression is novel and advantageous because it can provide a more specific targeting and effectiveness in modulating the immune response of T-cells.

### DETAILED DESCRIPTION OF THE INVENTION

By the term “suppressor or inhibitor” or a “molecule which (selectively) suppresses or inhibits” it is meant a molecule that effects a change in the expression of the target. The change is relative to the normal or baseline level of expression in the absence of the “suppressor or inhibitor” or of the molecule, but otherwise under similar conditions, and it represent a decrease in the normal/baseline expression. The suppression or inhibition of the expression of the target may be assessed by any means known to the skilled in the art. The assessment of the expression level or of the presence of the target is preferably performed using classical molecular biology techniques such as (real time Polymerase Chain Reaction) qPCR, microarrays, bead arrays, RNAse protection analysis or Northern blot analysis or cloning and sequencing. In the context of the present invention, the target is the gene, the mRNA, the cDNA, or the encoded protein thereof. The above described molecules also include salts, solvates or prodrugs thereof. The above described molecules may be or not solvated by H₂O. In the context of the present invention the term “targeting” or “complementary” may be intended as being fully or partly complementary to all of or part of the target sequence or as being capable of hybridizing to all or part of specific target sequence.

The polynucleotides as above described, as e.g. the siRNAs, may further comprise dTdT or UU 3′-overhangs, and/or nucleotide and/or polynucleotide backbone modifications as described elsewhere herein. In the context of the present invention, the term “polynucleotide” includes DNA molecules (e.g., cDNA or genomic DNA) and RNA molecules (e.g., mRNA, siRNA, shRNA) and analogs of the DNA or RNA generated using nucleotide analogs. The polynucleotide may be single-stranded or double-stranded. The RNA inhibitors as above defined are preferably capable of hybridizing to all or part of specific target sequence. Therefore, RNA inhibitors may be fully or partly complementary to all of or part of the target sequence. The RNA inhibitors may hybridize to the specified target sequence under conditions of medium to high stringency. An RNA inhibitor may be defined with reference to a specific sequence identity to the reverse complement of the sequence to which it is intended to target. The antisense sequences will typically have at least about 75%, preferably at least about 80%, at least about 85%, at least about 90%, at least about 95% or at least about 99% sequence identity with the reverse complements of their target sequences.

The term polynucleotide and polypeptide also include derivatives and functional fragments thereof. The polynucleotide may be synthesized using oligonucleotide analogs or derivatives (e.g., inosine or phosphorothioate nucleotides).

The molecule according to the invention may be an antibody or derivatives thereof.

The term gene herein also includes corresponding orthologous or homologous genes, isoforms, variants, allelic variants, functional derivatives, functional fragments thereof. The expression “protein” is intended to include also the corresponding protein encoded from a corresponding orthologous or homologous genes, functional mutants, functional derivatives, functional fragments or analogues, isoforms thereof.

In the context of the present invention, the term “polypeptide” or “protein” includes:

i. the whole protein, allelic variants and orthologs thereof;

ii. any synthetic, recombinant or proteolytic functional fragment;

iii. any functional equivalent, such as, for example, synthetic or recombinant functional analogues.

In the present invention “functional mutants” of the protein are mutants that may be generated by mutating one or more amino acids in their sequences and that maintain their activity. Indeed, the protein of the invention, if required, can be modified in vitro and/or in vivo, for example by glycosylation, myristoylation, amidation, carboxylation or phosphorylation, and may be obtained, for example, by synthetic or recombinant techniques known in the art. The term “derivative” as used herein in relation to a protein means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like. As used herein, the term “derivatives” also refers to longer or shorter polypeptides having e.g. a percentage of identity of at least 41%, preferably at least 41.5%, 50%, 54.9%, 60%, 61.2%, 64.1%, 65%, 70% or 75%, more preferably of at least 85%, as an example of at least 90%, and even more preferably of at least 95% with the herein disclosed genes and sequences, or with an amino acid sequence of the correspondent region encoded from orthologous or homologous gene thereof. The term “analogue” as used herein referring to a protein means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. A “derivative” may be a nucleic acid molecule, as a DNA molecule, coding the polynucleotide as above defined, or a nucleic acid molecule comprising the polynucleotide as above defined, or a polynucleotide of complementary sequence. In the context of the present invention the term “derivatives” also refers to longer or shorter polynucleotides and/or polynucleotides having e.g. a percentage of identity of at least 41%, 50%, 60%, 65%, 70% or 75%, more preferably of at least 85%, as an example of at least 90%, and even more preferably of at least 95% or 100% with e.g. SEQ ID NO: 1-12 or with their complementary sequence or with their DNA or RNA corresponding sequence. The term “derivatives” and the term “polynucleotide” also include modified synthetic oligonucleotides. The modified synthetic oligonucleotide are preferably LNA (Locked Nucleic Acid), phosphoro-thiolated oligos or methylated oligos, morpholinos, 2′-O-methyl, 2′-O-methoxyethyl oligonucleotides and cholesterol-conjugated 2′-O-methyl modified oligonucleotides (antagomirs). The term “derivative” may also include nucleotide analogues, i.e. a naturally occurring ribonucleotide or deoxyribonucleotide substituted by a non-naturally occurring nucleotide. The term “derivatives” also includes nucleic acids or polypeptides that may be generated by mutating one or more nucleotide or amino acid in their sequences, equivalents or precursor sequences. The term “derivatives” also includes at least one functional fragment of the polynucleotide. In the context of the present invention “functional” is intended for example as “maintaining their activity”. As used herein “fragments” refers to polynucleotides having preferably a length of at least 200, 400, 600, 800, 1000 nucleotides, 1100 nucleotide, 1200 nucleotides, 1300 nucleotides, 1400 nucleotides, 1500 nucleotides or to polypeptide having preferably a length of at least 50 aa, 100 aa, 150 aa, 200 aa, 250 aa, 300 aa. The term “polynucleotide” also refers to modified polynucleotides. As used herein, the term “vector” refers to an expression vector, and may be for example in the form of a plasmid, a viral particle, a phage, etc. Such vectors may include bacterial plasmids, phage DNA, baculovirus, yeast plasmids, vectors derived from combinations of plasmids and phage DNA, viral DNA such as vaccinia, adenovirus, lentivirus, fowl pox virus, and pseudorabies. Large numbers of suitable vectors are known to those of skill in the art and are commercially available. The polynucleotide sequence, preferably the DNA sequence in the vector is operatively linked to an appropriate expression control sequence(s) (promoter) to direct mRNA synthesis. As representative examples of such promoters, one can mention prokaryotic or eukaryotic promoters such as CMV immediate early, HSV thymidine kinase, early and late SV40, LTRs from retrovirus, and mouse metallothionein-I. The expression vector may also contain a ribosome binding site for translation initiation and a transcription vector. The vector may also include appropriate sequences for amplifying expression. In addition, the vectors preferably contain one or more selectable marker genes to provide a phenotypic trait for selection of transformed host cells such as dihydro folate reductase or neomycin resistance for eukaryotic cell culture, or such as tetracycline or ampicillin resistance in E. coli. As used herein, the term “host cell genetically engineered” relates to host cells which have been transduced, transformed or transfected with the polynucleotide or with the vector described previously. As representative examples of appropriate host cells, one can cite bacterial cells, such as E. coli, Streptomyces, Salmonella typhimurium, fungal cells such as yeast, insect cells such as Sf9, animal cells such as CHO or COS, plant cells, etc. The selection of an appropriate host is deemed to be within the scope of those skilled in the art from the teachings herein. Preferably, said host cell is an animal cell, and most preferably a human cell. The introduction of the polynucleotide or of the vector described previously into the host cell can be effected by method well known from one of skill in the art such as calcium phosphate transfection, DEAE-Dextran mediated transfection, electroporation, lipofection, microinjection, viral infection, thermal shock, transformation after chemical permeabilization of the membrane or cell fusion. The polynucleotide may be a vector such as for example a viral vector. The polynucleotides as above defined can be introduced into the body of the subject to be treated as a nucleic acid within a vector which replicates into the host cells and produces the polynucleotides. Suitable administration routes of the pharmaceutical composition of the invention include, but are not limited to, oral, rectal, transmucosal, intestinal, enteral, topical, suppository, through inhalation, intrathecal, intraventricular, intraperitoneal, intranasal, intraocular, parenteral (e.g., intravenous, intramuscular, intramedullary, and subcutaneous), chemoembolization. Other suitable administration methods include injection, viral transfer, use of liposomes, e.g. cationic liposomes, oral intake and/or dermal application. In certain embodiments, a pharmaceutical composition of the present invention is administered in the form of a dosage unit (e.g., tablet, capsule, bolus, etc.). For pharmaceutical applications, the composition may be in the form of a solution, e.g. an injectable solution, emulsion, suspension or the like. The carrier may be any suitable pharmaceutical carrier. Preferably, a carrier is used which is capable of increasing the efficacy of the molecules to enter the target cells. Suitable examples of such carriers are liposomes. In the pharmaceutical composition according to the invention, the suppressor or inhibitor may be associated with other therapeutic agents. The pharmaceutical composition can be chosen on the basis of the treatment requirements. Such pharmaceutical compositions according to the invention can be administered in the form of tablets, capsules, oral preparations, powders, granules, pills, injectable, or infusible liquid solutions, suspensions, suppositories, preparation for inhalation. A reference for the formulations is the book by Remington (“Remington: The Science and Practice of Pharmacy”, Lippincott Williams & Wilkins, 2000). The expert in the art will select the form of administration and effective dosages by selecting suitable diluents, adjuvants and/or excipients. Pharmaceutical compositions of the present invention may be manufactured by processes well known in the art, e.g., using a variety of well-known mixing, dissolving, granulating, levigating, emulsifying, encapsulating, entrapping or lyophilizing processes. The compositions may be formulated in conjunction with one or more physiologically acceptable carriers comprising excipients and auxiliaries which facilitate processing of the active compounds into preparations which can be used pharmaceutically. Proper formulation is dependent upon the route of administration chosen. Parenteral routes are preferred in many aspects of the invention. For injection, including, without limitation, intravenous, intramusclular and subcutaneous injection, the compounds of the invention may be formulated in aqueous solutions, preferably in physiologically compatible buffers such as physiological saline buffer or polar solvents including, without limitation, a pyrrolidone or dimethylsulfoxide. The compounds are preferably formulated for parenteral administration, e.g., by bolus injection or continuous infusion. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water-soluble form, such as, without limitation, a salt of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxyl ethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use. For oral administration, the compounds can be formulated by combining the active compounds with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl-methylcellulose, sodium carboxy-methylcellulose, and/or polyvinylpyrrolidone (PVP). For administration by inhalation, the molecules of the present invention can conveniently be delivered in the form of an aerosol spray using a pressurized pack or a nebulizer and a suitable propellant. The molecules may also be formulated in rectal compositions such as suppositories or retention enemas, using, e.g., conventional suppository bases such as cocoa butter or other glycerides. In addition to the formulations described previously, the compounds may also be formulated as depot preparations. Such long acting formulations may be administered by implantation (for example, subcutaneously or intramuscularly) or by intramuscular injection. The compounds of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt. Additionally, the compounds may be delivered using a sustained-release system, such as semi-permeable matrices of solid hydrophobic polymers containing the therapeutic agent. Various sustained-release materials have been established and are well known by those skilled in the art. A therapeutically effective amount refers to an amount of compound effective to prevent, alleviate or ameliorate the protein conformational disease. Determination of a therapeutically effective amount is well within the capability of those skilled in the art, especially in light of the disclosure herein. Generally, the amount used in the treatment methods is that amount which effectively achieves the desired therapeutic result in mammals. In particular, the molecules administration should follow the current clinical guidelines. A suitable daily dosage will range from 0.001 to 10 mg/kg body weight, in particular 0.1 to 5 mg/kg. In the case of polynucleotides, a suitable daily dosage may be in the range of 0.001 pg/kg body weight to 10 mg/kg body weight. Typically, the patient doses for parenteral administration of the molecules described herein range from about 1 mg/day to about 10,000 mg/day, more typically from about 10 mg/day to about 1,000 mg/day, and most typically from about 50 mg/day to about 500 mg/day. The range set forth above is illustrative and those skilled in the art will determine the optimal dosing of the compound selected based on clinical experience and the treatment indication.

As used herein, “oligonucleotides” or “polynucleotide” shall mean single or double stranded RNA or DNA, including ASOs, sgRNAs and siRNA capable of binding to complementary single or double stranded RNA or DNA target sequences. The sequence-specific portion of the therapeutic oligonucleotides that are ASOs or sgRNAs or siRNA of the invention comprise nucleotide sequences of from about 7 bases to about 45 bases in length. Additional bases that are not sequence-specific may be included in the oligonucleotides, such as for example linker sequence. By sequence-specific is meant the portion of the oligonucleotide that is complementary to the target RNA or DNA and/or directs cleavage of the target RNA or DNA.

As used herein, “ASOs” shall mean short stretches (about 7 to about 45 sequence-specific nucleotides) of DNA or derivatized DNA (e.g., phosphorothioated DNA) that contains sequence which is complementary to a target DNA or RNA. The complementary portion of the ASOs will typically range from about 30% to about 100% of the oligonucleotide.

As used herein, “siRNA” shall mean an RNA duplex in which each strand of the duplex contains between about 15 and about 30 bases in length, and wherein at least one of the strands shares at least about 90%, more preferably up to about 100% homology with a DNA or RNA target.

As used herein, “gene expression” shall mean mRNA synthesis or mRNA translation.

In one embodiment of the invention, therapeutic oligonucleotides of the invention are ASOs. ASOs encompass single-stranded DNA or RNA that is complementary to a portion of a specific RNA sequence, or alternatively the complementary gene sequence, and reduce or inhibit gene expression. Non-limiting examples of ASOs include RNA sequences complementary to an mRNA transcript, thereby forming an RNA duplex resulting in reduced levels of translation. Alternatively, an ASO may encompass a DNA sequence complementary to an mRNA transcript, which hybridizes with the mRNA transcript and serves as a substrate for RNaseH.

The technology of antisense oligonucleotides has been known in the art as a promising source of therapeutics. Antisense oligonucleotides rely upon Watson-Crick base pairing between a known nucleic acid sequence and its reverse complement to inhibit gene expression (Jen, K., et al, Stem Cells, 18:307-19 (2000)). Antisense oligonucleotide therapy can be utilized to combat a wide range of disorders, for example the expression of human genes involved in diseases or disorders, or alternatively by targeting the replication of infectious agents (Tanaka, M., et al, Respir. Res., 2:5-9 (2000); Bunnell, B. A., et al, Clin. Micro. Rev., 11:42-56 (1998)). Crucial considerations which must be addressed when designing antisense oligonucleotide therapies include antisense stability in vivo, effective delivery of the antisense oligonucleotide therapeutic, and efficient intracellular localization of the antisense oligonucleotide (Jen, K., et al, Stem Cells, 18:307-19 (2000)).

It is well known that, depending on the target gene, ASOs which hybridize to any part of the target gene, such as coding regions, introns, the 5′ untranslated region (5′UTR), translation initiation site, or 3′UTR may have therapeutic utility. Therefore, the sequences listed herein are merely exemplary of the possible therapeutic oligonucleotides that may be used with the invention, which include all of the ASOs known in the art. Furthermore, all of the alternative nucleic acid chemistries proposed in the art can be used with the invention although the degree of effectiveness may vary. Chemistries applicable with the therapeutic oligonucleotides of the invention are discussed in further detail in the section entitled “Conjugation Chemistry and Carrier Molecules” provided infra. In short, the compounds listed herein represent the broad class of therapeutic oligonucleotides of various chemistries which are useful with this invention. In one embodiment of the invention, the sequence-binding portion of ASO and siRNA therapeutic oligonucleotides of the invention is about 7 to about 45 bases in length. In a preferred embodiment of the invention, the sequence-binding portion of ASO and siRNA therapeutic oligonucleotides of the invention is about 10 to about 30 nucleotides in length. In a particularly preferred embodiment of the invention, the sequence-binding portion of ASO and siRNA therapeutic oligonucleotides of the invention is about 15 to about 25 nucleotides in length. Additional oligonucleotides which are useful in the invention include oligonucleotides previously demonstrating efficacy in free form in the art.

Therapeutic oligonucleotides of the invention also encompass siRNA. siRNA derive from RNA interference, which is a natural cellular process for silencing the transcription of certain genes (Sharp, P. A., Genes & Dev., 15:485-490 (2001); Carmichael, G. G., Nature, 418:379-380 (2002)). siRNA associate with cellular protein complexes and direct cleavage of complementary target RNAs by those protein complexes.

In the present invention, siRNA encompass duplex RNAs of approximately 15-30 bases in length, one strand of the duplex RNA preferably having at least about 90% homology with a RNA target, more preferably having up to about 100% homology with a RNA target. Alternatively, siRNAs share enough homology with a RNA target to direct cleavage of complementary target RNA by protein complexes. Homology between two nucleotide sequences can be determined by one of ordinary skill in the art using search-based computer programs, such as the BLAST or FASTA programs. Alternatively, one of ordinary skill in the art can determine sequence homology using sequence alignment programs such as MegAlign (contained within the DNASTAR suite of computer programs).

siRNAs are modified with chemical reactive groups described infra, enabling the formation of covalent bonds with mobile proteins, preferably human serum albumin, in a preferred embodiment of the invention, modification of the siRNA duplex through addition of a chemical reactive group occurs at a terminus. Chemical modification of the RNA duplex with a chemical reactive group may occur at any of the 4 termini of the RNA duplex, either the 5′ or 3′ termini of either of the two RNA strands of the RNA duplex.

Preferably, the inhibitory nucleic acid comprises one or more peptide nucleic acid (PNA) or locked nucleic acid (LNA) molecules or the inhibitory nucleic acid is a ribonucleic acid analogue comprising a ribose ring having a bridge between its 2′-oxygen and 4′-carbon.

Preferably, the ribonucleic acid analogue comprises a methylene bridge between the 2′-oxygen and the 4′-carbon.

Preferably, at least one nucleotide of the inhibitory nucleic acid comprises a modified sugar moiety selected from a 2′-O-methoxyethyl modified sugar moiety, a 2′-methoxy modified sugar moiety, a 2′-O-alkyl modified sugar moiety, and a bicyclic sugar moiety.

Preferably the inhibitory nucleic comprises at least one modified internucleoside linkage selected from phosphorothioate, phosphorodithioate, alkylphosphonothioate, phosphoramidate, carbamate, carbonate, phosphate triester, acetamidate, carboxymethyl ester, and combinations thereof.

L1 subfamily comprises: HAL1, HAL1B, HAL1M8, IN25, L1, L1HS, L1M1_5, L1M1B_5, L1M2A_5, L1M3A_5, L1M3B_5, L1M3C_5, L1M3D_5, L1M3DE_5, L1M4B, L1M6_5end, L1M6B_5end, L1M7_5end, L1MA1, L1MA10, L1MA2, L1MA3, L1MA4, L1MA4A, L1MA5 L1MA5A, L1MA6, L1MA7, L1MA8, L1MA9, L1MB1, L1MB2, L1MB3, L1MB3_5, L1MB4 L1MB5, L1MB6_5, L1MB7, L1MB8, L1MC1, L1MC2, L1MC4, L1MCB_5, L1MD1, L1MD2, L1MDB_5, L1ME_ORF2, L1ME1, L1ME2, L1ME3, L1ME3A, L1ME4A, L1MEA_5, L1MEC_5, L1MED5, L1MEf_5end, L1PA10, L1PA11, L1PA12, L1PA12_5, L1PA13, L1PA13_5, L1PA14, L1PA15, L1PA16, L1PA2, L1PA3, L1PA4, L1PA5, L1PA6, L1PA7, L1PA8, L1PB1, L1PB2, L1PB2c, L1PB3, L1PB4, L1PREC1, L1PREC2. (https://www.girinst.org/repbase/, Kenji K. Kojima, Human transposable elements in Repbase: genomic footprints from fish to humans, Mob DNA. 2018; 9: 2).

In the context of the present invention, cancer or tumour may include any time of cancer or tumours, e.g. lung cancer, preferably non-small cells lung carcinoma (NSCLC), colorectal cancer (CRC), intestine tumour or melanoma.

The invention will be now illustrated by means of non-limiting examples referring to the following figures.

FIG. 1. Quiescent naïve CD4⁺ T-cells are enriched by LINE1 RNAs that are downregulated upon TCR activation by mTORC1 in vitro and in vivo.

(a) Representative confocal fluorescence microscopy images of LINE1 RNA FISH (red) performed on quiescent naïve and memory CD4+ and CD8+ T-cells. As control, naïve CD4+ T-cells were treated with RNAse. Original magnification 63×. Scale bar 5 μm. (b) Violin plot representation of LINE1 RNA FISH signal from four individuals; at least 246 nuclei per group were assessed. *** P<0.0001 Ordinary One-way ANOVA. (c) LINE1 expression levels by qRT-PCR in quiescent naïve and memory Th1, Th2, Th17 CD4+ T-cells and in quiescent naïve and memory CD8+ T-cells; each dot represents a different donor. *** P<0.0001 Ordinary One-way ANOVA. (d) Abundance of LINE1, HERV and Alu RNAs, in the cytoplasm, nucleoplasm and chromatin of quiescent naïve CD4+ T-cells from three individuals. Data are represented as mean. *P=0.0217 Ordinary One-way ANOVA. (e) LINE1 and 3 Actin expression levels by qRT-PCR in quiescent naïve CD4+ T-cells that have been treated with or without Actinomycin D (n=3 individuals). Data are represented as mean and ±s.e.m. 3 Actin Untreated vs Actinomycin D treated *P=0.046 One-tailed paired t test. (f) LINE1 expression levels by qRT-PCR in naïve CD4+ T-cells and activated at 2, 4 and 8 hours, 1, 3, 5, 7 days with TCR engagement and Th1 cytokines cocktail (n=6 individuals). *** P<0.0001 Ordinary One-way ANOVA. (g) Schematic representation of the signaling pathways downstream TCR activation. Drugs used to inhibit the pathways and their molecular targets are indicated. (h) LINE1 expression levels by qRT-PCRs in quiescent naïve CD4+ T-cells that were activated with TCR engagement and treated with different signaling pathway inhibitors for 8 hours after activation (n=4 individuals); each dot represents a different donor. Control vs Rapamycin *** P=0.0003 Two-tailed paired t test. (i) Representative confocal fluorescence microscopy images of LINE1 RNA FISH (red) performed on memory Th1 CD4⁺ T-cells isolated from healthy individuals, transplanted patients treated with Everolimus and LAM patients treated with Sirolimus. Original magnification 63×. Scale bar 5 μm. (j) Violin plot representation of LINE1 RNA FISH signal from memory Th1 CD4⁺ T-cells of two individuals per group; at least 138 nuclei per group were assessed. *** P<0.0001 Mann Whitney t test, ** P=0.0058 Mann Whitney t test. (k) LINE1 expression levels by qRT-PCRs in memory CD4⁺ T-cells isolated from four healthy individuals, two transplanted patients treated with Everolimus and four LAM patients treated with Sirolimus. Data are represented as mean and ±s.e.m. ** P=0.007, Ordinary One-way ANOVA.

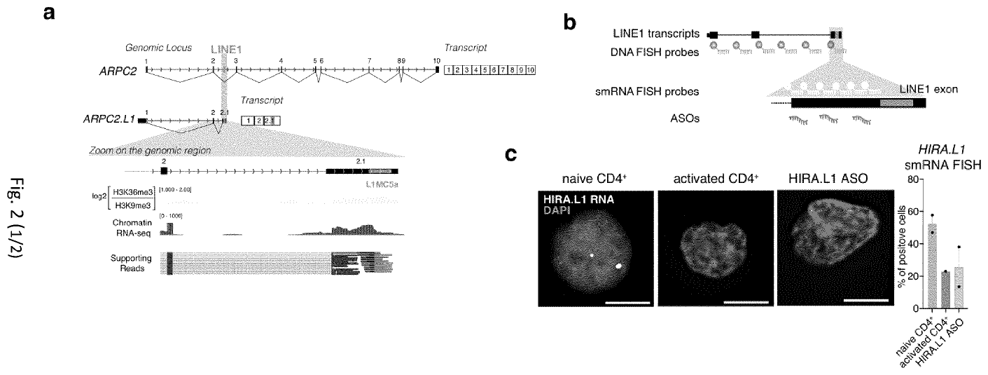

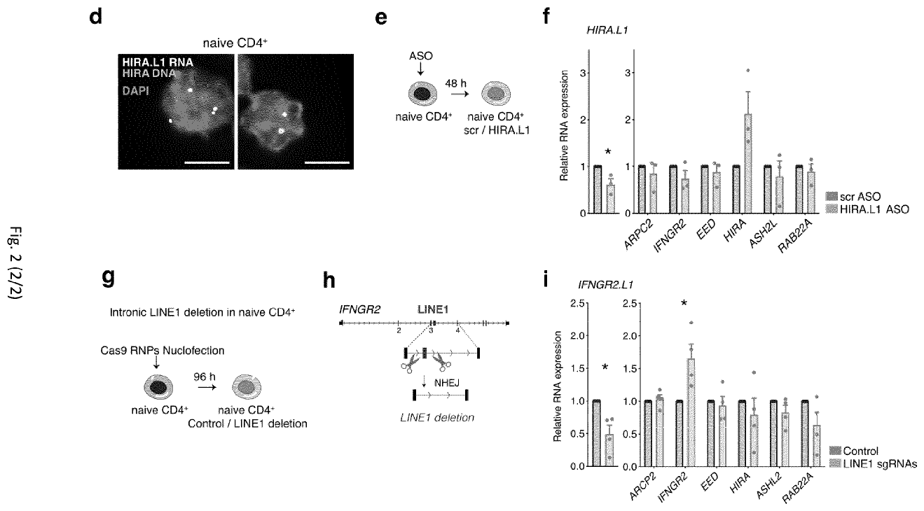

FIG. 2. LINE1 are spliced in non-canonical transcripts variants of cellular activation genes that regulate the transcription of the corresponding genes loci.

(a) ARCP2.L1 are shown as an example of LINE1 containing transcript: the novel exon containing LINE1 element is zoomed. Tracks for H3K36me3 vs H3K9me3 log fold change (dark red), coverage tracks of naïve CD4⁺ T-cells chromatin RNAseq (blue), split and supporting reads for novel exon are shown. (b-c) Schematic representation of DNA FISH probes (green), smRNA FISH probes (pink) and ASOs (blue) position in LINE1 transcripts sequence. Representative widefield fluorescence microscopy images of smRNA FISH for (c) HIRA.L1 performed on quiescent and activated naïve CD4⁺ T-cells and on naïve CD4⁺ T-cells that have been treated for 48 hours with HIRA.L1 ASOs or control (Scr) ASOs. Original magnification 100×. Scale bar 5 μm. Right, bar plot representing number of dots per nuclei. (d) Left, representative widefield fluorescence microscopy images of TSA RNA FISH on HIRA.L1 (red) combined with DNA FISH for HIRA genomic locus (green) performed on quiescent naïve CD4⁺ T-cells. Original magnification 100×. Scale bar 5 μm. (e) Schematic representation of HIRA.L1 knock down in quiescent naïve CD4+ T-cells. Naïve CD4⁺ T-cells were treated with HIRA.L1 or control (Scr) ASOs for 48 hours. (f) HIRA.L1 and Canonical transcripts expression levels by qRT-PCR in quiescent naïve CD4⁺ T-cells that have been treated for 48 hours with HIRA.L1 or control (Scr) ASOs (n=3 individuals). * P=0.04, Two-tailed paired t test. (g) Schematic representation of LINE1 transcripts deletion with Cas9/RNP in quiescent naïve CD4⁺ T cells. Naïve CD4⁺ T cells were nucleofected with Cas9/RNP and kept in culture for 96 hours. (h-i) Schematic representation of IFNGR2 (h) sequence depicting the location of sgRNA targeting IFNGR2 locus. (i) IFNGR2.L1 and Canonical transcripts expression level by qRT-PCRs in quiescent naïve CD4⁺ T cells 96 hours after nucleofection with Cas9/RNP for IFNGR2.L1 or control (n=4 individuals). LINE1 transcripts, IFNGR2.L1 * P=0.04 Two-tailed paired t test, Canonical transcripts * P=0.0275 One-tailed paired t test.

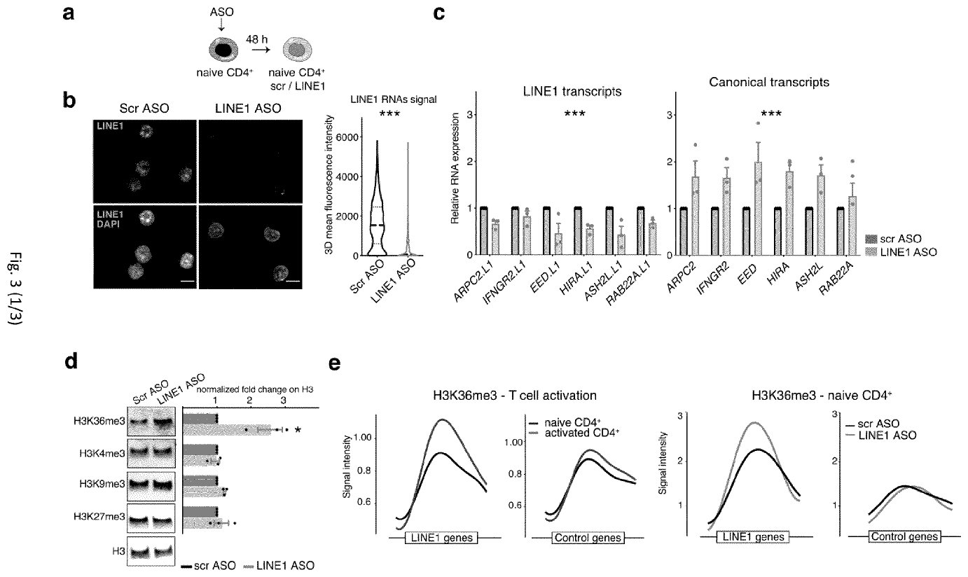

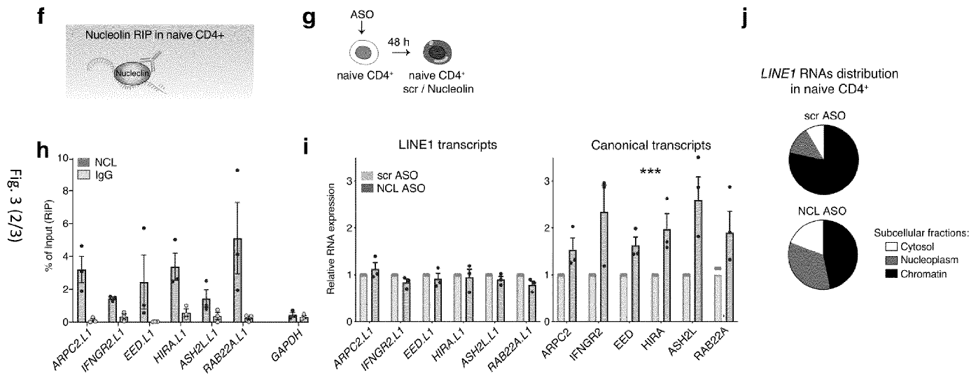

FIG. 3. LINE1 transcripts in complex with Nucleolin keep paused expression of cell activation genes hampering H3K36me3 deposition in quiescent naïve CD4+ T-cells.

(a) Schematic representation of LINE1 RNAs knock down in quiescent naïve CD4⁺ T-cells. Naïve CD4⁺ T-cells were treated with LINE1 or control (Scr) ASOs for 48 hours. (b) Left, representative confocal fluorescence microscopy images of LINE1 RNA FISH (red) performed on quiescent naïve CD4⁺ T-cells that have been treated for 48 hours with LINE1 or control (Scr) ASOs. Original magnification 63×. Scale bar 5 μm. Right, violin plot representation of LINE1 RNA FISH signal from two healthy individuals; at least 500 nuclei per group were assessed *** P<0.001 Mann Whitney t test (c) LINE1 transcripts and Canonical transcripts expression levels by qRT-PCRs in quiescent naïve CD4⁺ T-cells that have been treated for 48 hours with LINE1 or control (Scr) ASOs (n=3 individuals). Data are represented as mean and ±s.e.m. LINE1 transcripts *** P<0.0001, F=68.60 Two-way ANOVA; Canonical transcripts*** P<0.0001, F=39.39. Two-way ANOVA. (d) H3K36me3, H3K4me3, H3K9me3, H3K27me3 levels were assessed by quantitative western blot in quiescent naïve CD4⁺ T-cells that have been treated for 48 hours with LINE1 or control (Scr) ASOs (n=3 individuals). H3 was used as loading control. Data are represented as mean and ±s.e.m. H3K36me3 Scr vs LINE1 *P=0.0495 Two-tailed paired t test. (e) Positional distribution of H3K36me3 ChTP seq signal plotted on LINE1 containing genes or control genes bodies compared between naïve and activated CD4⁺ T-cells and quiescent naïve CD4+ T-cells that have been treated for 48 hours with LINE1 or control (Scr) ASOs. The median of H3K36me3 signal (ChIP/Input fold enrichment) across the genes is plotted. (f-g) LINE1 transcripts and control gene (GAPDH) were amplified by qRT-PCRs in Nucleolin RIP experiments performed in quiescent naïve CD4⁺ T-cells (n=3 individuals). Data are represented as mean % of input±s.e.m. (h) Schematic representation of Nucleolin knock down in quiescent naïve CD4⁺ T-cells. Naïve CD4⁺ T-cells were treated with Nucleolin or control (Scr) ASOs for 48 hours. (i) LINE1 transcripts and Canonical transcripts expression levels by qRT-PCRs in quiescent naïve CD4⁺ T-cells that have been treated for 48 hours with Nucleolin ASOs or control (Scr) ASOs (n=3 individuals). Canonical transcripts, *** P<0.0001, F=42.57.60 Two-way ANOVA. (j) Abundance of LINE1 RNAs in the cytoplasm, nucleoplasm and chromatin of quiescent naïve CD4⁺ T-cells that have been treated for 48 hours with Nucleolin or control (Scr) ASOs (n=3 individuals). Data are represented as mean and ±s.e.m. * P=0.0345, F=8.772 Two-way ANOVA. (k) Schematic representation of LINE1 RNAs knock down in activated CD4⁺ T-cells. Naïve CD4+ T-cells were treated with LINE1 or control (Scr) ASOs for 48 hours and and then activated for 7 days via TCR engagement in the presence of Th1 cytokine cocktail. (1) T-bet and IFNγ positive cells measured by intracellular staining in naïve CD4⁺ T-cells that have been treated with LINE1 or control (Scr) ASOs (n=8 individuals). Data are represented as mean and ±s.e.m. Tbet *** P=0.0009 Two-tailed paired t test; IFNγ *** P=0.0002 Two-tailed paired t test. (m) Schematic representation of Nucleolin knock down in activated CD4⁺ T-cells. Naïve CD4⁺ T-cells were treated with Nucleolin or control (Scr) ASOs for 48 hours and then activated for 7 days via TCR engagement in the presence of Th1 cytokine cocktail. (n) T-bet and IFNγ positive cells measured by intracellular staining in naïve CD4⁺ T-cells that have been treated with Nucleolin or control (Scr) ASOs for 48 hours, and then activated for 7 days via TCR engagement in the presence of Th1 cytokine cocktail (n=4 individuals). Data are represented as mean and ±s.e.m. Tbet * P=0.0142 Two-tailed paired t test; IFNγ ** P=0.0041 Two-tailed paired t test.

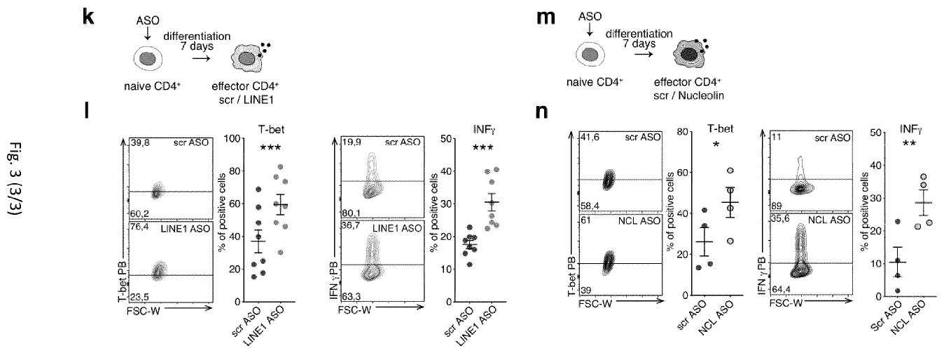

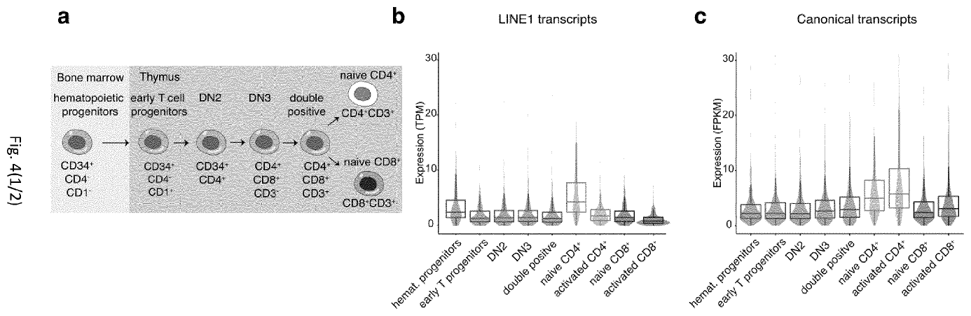

FIG. 4. LINE1 transcripts are under the control of the transcription factor IRF4.

(a) Schematic representation of CD4⁺ and CD8⁺ T-cell development in the Thymus. Surface markers specific for hematopoietic progenitors, early T cell progenitors, DN2, DN3, double positive, naïve CD4⁺ and naïve CD8⁺ are described. (b-c) Expression level both of the LINE1 transcripts and of the Canonical transcripts in RNA-seq datasets of progenitors, naïve and activated CD4⁺ T-cells and naïve and activated CD8⁺ T-cells. *** P<0.001 Wilcoxon rank sum test was done for every cell type in comparison to naïve CD4⁺ T-cells (paired option only in comparison with CD4+ activated T). (d) IRF4 levels were assessed by quantitative western blot in naïve CD4⁺ and naïve CD8⁺ T-cells. H3 is used as loading control. Data are represented as mean and ±s.e.m. n=3 individuals **P=0.0085 Two-tailed paired t test. (e) LINE1 containing genes and control gene (HECW1) promoters were amplified by qRT-PCRs in IRF4 ChTP experiments performed in quiescent naïve CD4⁺ T-cells and naïve CD8⁺ T-cells (n=3 individuals). Data are represented as mean % of input±s.e.m. LINE1 containing genes promoter ** P=0.0034, F=10.7, Two-way ANOVA. (f) Schematic representation of IRF4 knock down in quiescent naïve CD4⁺ T-cells. Naïve CD4⁺ T-cells were treated with IRF4 or control (Scr) ASOs for 48 hours. (g) LINE1 transcripts, Canonical transcripts and control gene (HECW1) expression levels by qRT-PCRs in quiescent naïve CD4⁺ T-cells that have been treated for 48 hours with IRF4 or control (Scr) ASOs (n=3 individuals). Data are represented as mean and ±s.e.m. LINE1 transcripts *** P=0.001, F=924 Two-way ANOVA; Canonical transcripts * P=0.04, F=22.2. Two-way ANOVA.

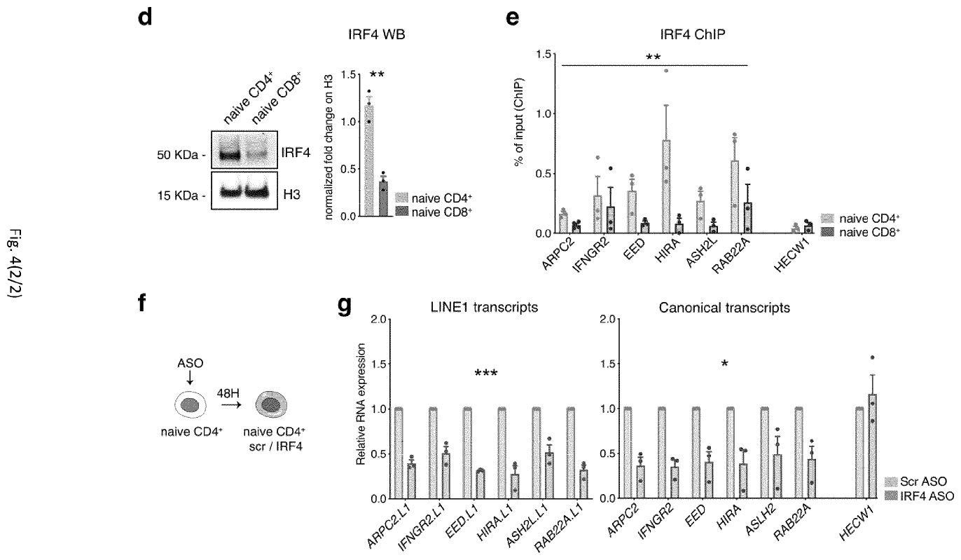

FIG. 5. Upon activation LINE1 transcripts are downregulated by the repressive splicing proteins PTBP1/MATR3 while the canonical transcripts expression is favored by the transcription factor GTF2F1.

(a-b) LINE1 transcripts and control gene (GAPDH) were amplified by qRT-PCRs in PTBP1 RIP experiments performed in quiescent naïve CD4⁺ T-cells and naïve CD4⁺ T-cells activated for 16 hours with TCR engagement and Th1 cytokines cocktail (n=3 individuals). Data are represented as mean % of input±s.e.m. *** P=0.0002, F=26.26, Two-way ANOVA. (c-d) LINE1 transcripts and control gene (GAPDH) were amplified by qRT-PCRs in GTF2F1 RIP experiments performed in quiescent naïve and naïve CD4⁺ T-cells activated for 16 hours with TCR engagement and Th1 cytokines cocktail (n=3 individuals). Data are represented as mean % of input±s.e.m. ** P=0.0014, F=16.68, Two-way ANOVA. (e) Schematic representation of the qRT-PCR assay on PTBP1 RIP (panel f) and on GTF2F1 RIP (panel g) to define whether PTBP1 or GTF2F1 is bound to canonical RAB22A mRNA, RAB22A.L1 or pre-mRNA in activated CD4⁺ T-cells. Primers are design to amplify i) LINE1 exon, ii) pre-mRNA in a region overlapping intron and nearby LINE1 exon, ii) spliced LINE1 transcript (fw primer on exon 2 and rev primer on LINE1 exon 2.1) and the iv) canonical transcript. (f) RAB22A RNA species were amplified by qRT-PCRs in PTBP1 RIP experiments performed in quiescent naïve CD4⁺ T-cells and naïve CD4⁺ T-cells activated for 16 hours with TCR engagement and Th1 cytokines cocktail (n=3 individuals). Data are represented as mean % of input±s.e.m. (g) RAB22A RNA species were amplified by qRT-PCRs in PTBP1 RIP experiments performed in quiescent naïve CD4⁺ T-cells and naïve CD4⁺ T-cells activated for 16 hours with TCR engagement and Th1 cytokines cocktail (n=3 individuals). Data are represented as mean % of input±s.e.m. (h) Schematic representation of PTBP1 and GTF2F1 knock down in activated CD4⁺ T-cells. Naïve CD4⁺ T-cells were treated with PTBP1 and GTF2F1 or control (Scr) ASOs for 48 hours and then activated for 16 hours via TCR engagement in the presence of Th1 cytokine cocktail. (i) LINE1 transcripts and Canonical transcripts expression levels by qRT-PCRs in activated CD4⁺ T-cells that have been treated for 48 hours with PTBP1 and GTF2F1 or control (Scr) ASOs and then activated for 16 hours via TCR engagement in the presence of Th1 cytokine cocktail (n=3 individuals). LINE1 transcripts, *** P=0.0006, F=14.89, Two-way ANOVA; Canonical transcripts, *** P<0.0001, F=44.52, Two-way ANOVA.

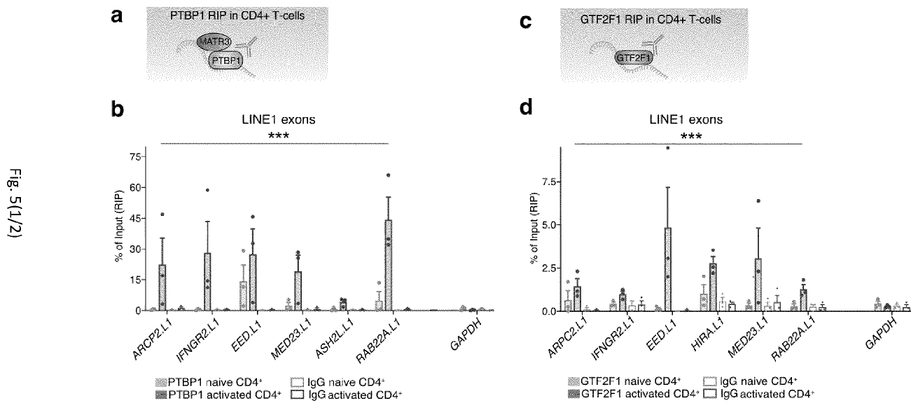

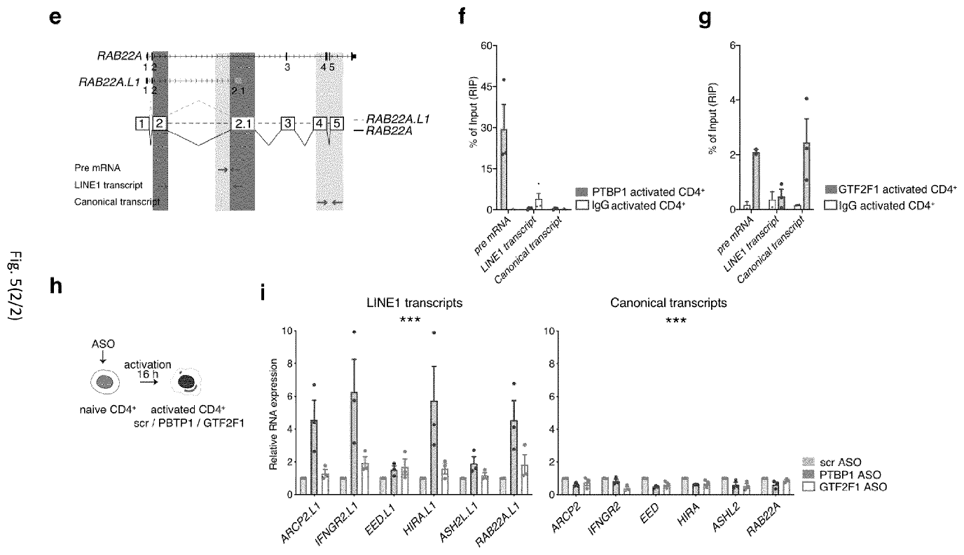

FIG. 6. LINE1 transcripts re-accumulate in dysfunctional tumor infiltrating effector lymphocytes.

(a) Top, representative confocal fluorescence microscopy images of LINE1 RNA FISH (red) performed on memory CD4⁺ and CD8⁺ T-cells infiltrating normal adjacent tissue or CRC tumor. Original magnification 63×. Scale bar 5 μm. Below, violin plot representation of LINE1 RNA FISH signal from two patients; at least 100 nuclei per group were assessed. Memory CD4⁺ T-cells *** P<0.001 Two-tailed Mann-Whitney Test; memory CD8⁺ T-cells *** P<0.001 Two-tailed Mann-Whitney Test. (b) Representative confocal fluorescence microscopy images of LINE1 RNA FISH (red) performed on memory CD4⁺ and CD8⁺-T-cells infiltrating normal adjacent tissue or NSCLC tumor. Original magnification 63×. Scale bar 5 μm. Below, violin plot representation of LINE1 RNA FISH signal from three patients for CD4⁺ T-cells and two patients for CD8⁺ T-cells; at least 84 nuclei per group were assessed. Memory CD4⁺ T-cells *** P<0.001 Two-tailed Mann-Whitney Test. (c) Left, representative confocal fluorescence microscopy images of LINE1 RNA FISH (red) performed on effector CD4⁺ and dysfunctional CD4⁺ T-cells. Original magnification 63×. Scale bar 5 μm. Right, violin plot representation of LINE1 RNA FISH signal from healthy individuals; at least 100 nuclei per group were assessed *** P<0.001 Mann Whitney t test. (d) LINE1 transcripts and Canonical transcripts expression levels by qRT-PCRs in effector CD4⁺ and dysfunctional CD4⁺ T-cells (n=3 individuals). Data are represented as mean and ±s.e.m. LINE1 transcripts ** P=0.0089, F=8.092 Two-way ANOVA; Canonical transcripts *** P<0.0001, F=38.08. Two-way ANOVA. (e) Left, representative confocal fluorescence microscopy images of LINE1 RNA FISH (red) performed on effector CD8⁺ and dysfunctional CD8⁺ T-cells. Original magnification 63×. Scale bar 5 μm. Right, violin plot representation of LINE1 RNA FISH signal from healthy individuals; at least 100 nuclei per group were assessed *** P<0.001 Mann Whitney t test. (f) LINE1 transcripts and Canonical transcripts expression levels by qRT-PCRs in effector CD8⁺ and dysfunctional CD8⁺ T-cells (n=3 individuals). Data are represented as mean and +s.e.m. LINE1 transcripts ** P=0.0039, F=17.93 Two-way ANOVA; Canonical transcripts *** P<0.0001, F=83.66. Two-way ANOVA.

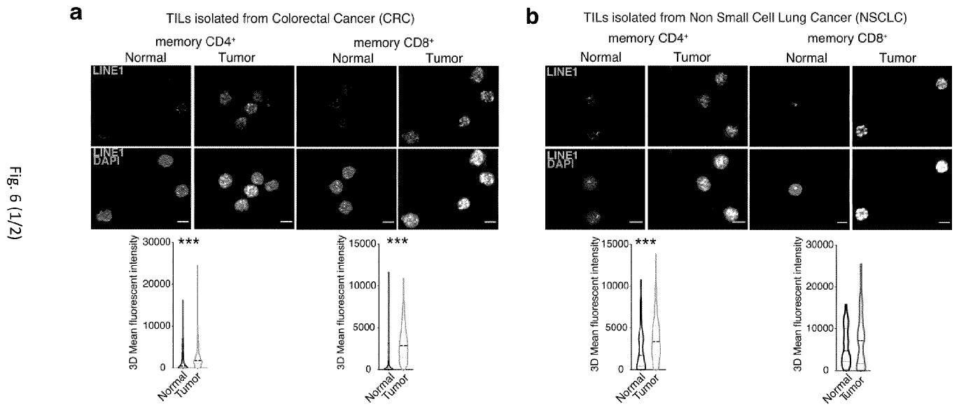

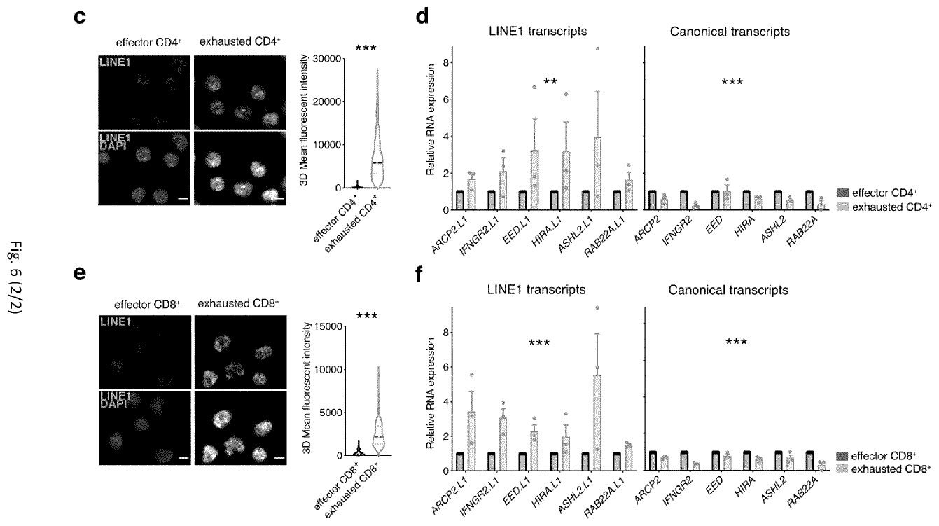

FIG. 7. LINE1 transcripts re-accumulation in dysfunctional effector lymphocytes is regulated by IRF4, stabilized at chromatin by Nucleolin and by the loss of GTF2F1/PTBP1 binding.

(a) IRF4, Nucleolin, GTF2F1 and PTBP1 levels were assessed by quantitative western blot in effector CD4⁺ and dysfunctional CD4⁺ T-cells and in effector CD8⁺ and dysfunctional CD8⁺ T-cells. H3 was used as loading control. Data are represented as mean and ±s.e.m (n=2 individuals). (b) RAB22A.L1, ARCP2.L1 and IFNGR2.L1 were amplified by qRT-PCRs in Nucleolin, PTBP1 and GTF2F1 RIP experiments performed in effector and dysfunctional CD4⁺ T-cells and effector and dysfunctional CD8⁺ T-cells (n=3 individuals). Data are represented as mean % of input+s.e.m. (c) Schematic representation of IRF4 knock down in dysfunctional CD4⁺ and CD8⁺ T-cells. Dysfunctional T-cells were treated with IRF4 or control (Scr) ASOs for 48 hours. (d) LINE1 transcripts and Canonical transcripts expression levels by qRT-PCRs in dysfunctional CD4⁺ T-cells treated for 48H with IRF4 or control (Scr) ASOs (n=3 individuals). Data are represented as mean and ±s.e.m. LINE1 transcripts *** P<0.001, F=47.6 Two-way ANOVA; Canonical transcripts *** P<0.0001, F=36.1. Two-way ANOVA. (e) LINE1 transcripts and Canonical transcripts expression by qRT-PCRs in dysfunctional CD8⁺ T-cells treated with IRF4 or control (Scr) ASOs (n=3 individuals). Data are represented as mean and ±s.e.m. LINE1 transcripts ** P=0.009, F=22.6 Two-way ANOVA; Canonical transcripts ** P=0.001, F=16.8. Two-way ANOVA.

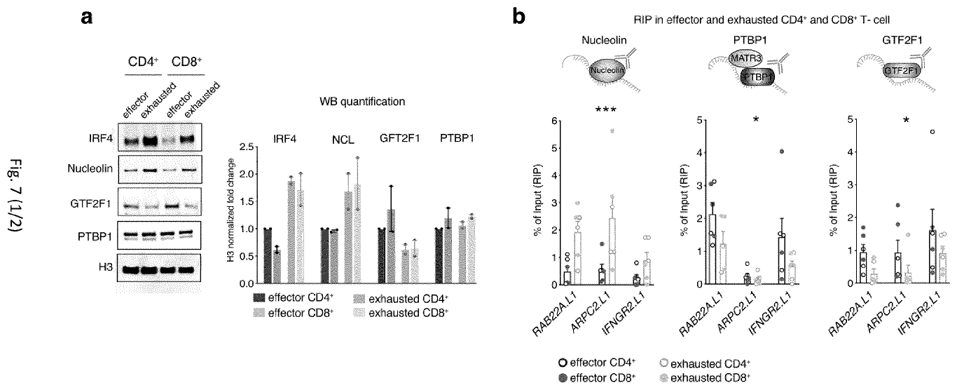

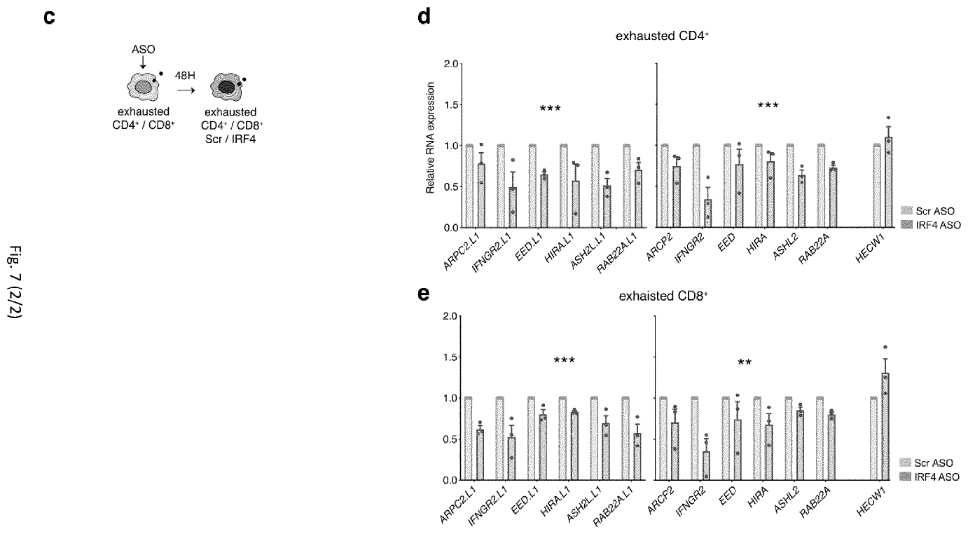

FIG. 8. LINE1 transcripts level modulates the dysfunctional phenotype of CD4+ and CD8+ memory T-cells infiltrating CRC or NSCLC.

(a) Schematic representation of the immunological assays performed on memory CD4+ and CD8+ memory T-cells infiltrating and isolated from CRC or NSCLC treated with LINE1 or control (Scr) ASO. After ASO treatment, tumor infiltrating memory CD4+ and CD8+(TILs) will be tested for Inhibitory Checkpoint staining (panel b and c), effector cytokines secretion (panel d and e) and the ability to kill heterologous antigen presenting cells with MHCII and MHCI as monocytes (panel f and g). (b) PD-1, TIM-3 or LAG-3 positive cells measured by surface markers staining in memory CD4+ T-cells isolated from CRC (black, n=3 individuals) or NSCLC (red, n=3 individuals) that have been treated with LINE1 or control (Scr) ASOs for 48 hours, PD-1 Scr ASO vs LINE ASO ** P=0.0044, Two-tailed paired t test; TIM-3 Scr ASO vs LINE ASO * P=0.017, Two-tailed paired t test; LAG-3 Scr ASO vs LINE ASO * P=0.04, Two-tailed paired t test. (c) PD-1, TIM-3 or LAG-3 positive cells measured by surface markers staining in memory CD8+ T-cells isolated from CRC (black, n=3 individuals) or NSCLC (red, n=3 individuals) that have been treated with LINE1 or control (Scr) ASOs for 48 hours, PD-1 Scr ASO vs LINE ASO * P=0.0268, Two-tailed paired t test; LAG-3 Scr ASO vs LINE ASO * P=0.03, Two-tailed paired t test. (d) IFNγ or GrzB positive cells measured by intracellular staining in memory CD4+ T-cells isolated from CRC (black, n=2 individual) or NSCLC (red, n=2 individual) that have been treated with LINE1 or control (Scr) ASOs for 48 hours and then activated by TCR engagement for additional 48 hours. Data are represented as mean and ±s.e.m. IFNγ Scr ASO vs LINE ASO * P=0.04, One-tailed paired t test; GrzB Scr ASO vs LINE ASO * P=0.02, One-tailed paired t test (e) IFNγ, GrzB or PerfA positive cells measured by intracellular staining in memory CD8+ T-cells isolated from CRC (black, n=3 individual) or NSCLC (red, n=1 individual) that have been treated with LINE1 or control (Scr) ASOs for 48 hours and then activated by TCR engagement for additional 48 hours. Data are represented as mean and ±s.e.m. IFNγ Scr ASO vs LINE ASO ** P=0.0095, Two-tailed paired t test; GrzB Scr ASO vs LINE ASO * P=0.03, One-tailed paired t test; PerfA Scr ASO vs LINE ASO * P=0.035, One-tailed paired t test. (f-g) Percentages of dead heterologous Monocytes co-cultured for 12 hours with memory CD4+ (f) or CD8+ (g) T-cells from CRC (black, n=1 individual) or NSCLC (red, n=2 individual) that have been treated with LINE1 or control (Scr) ASOs for 48 hours. Memory CD4+ T-cells * P=0.02, Two-tailed paired t test; memory CD8+ T-cells * P=0.04, Two-tailed paired t test.

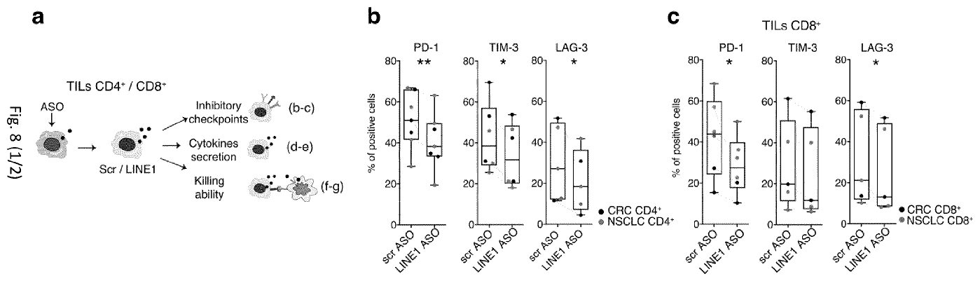

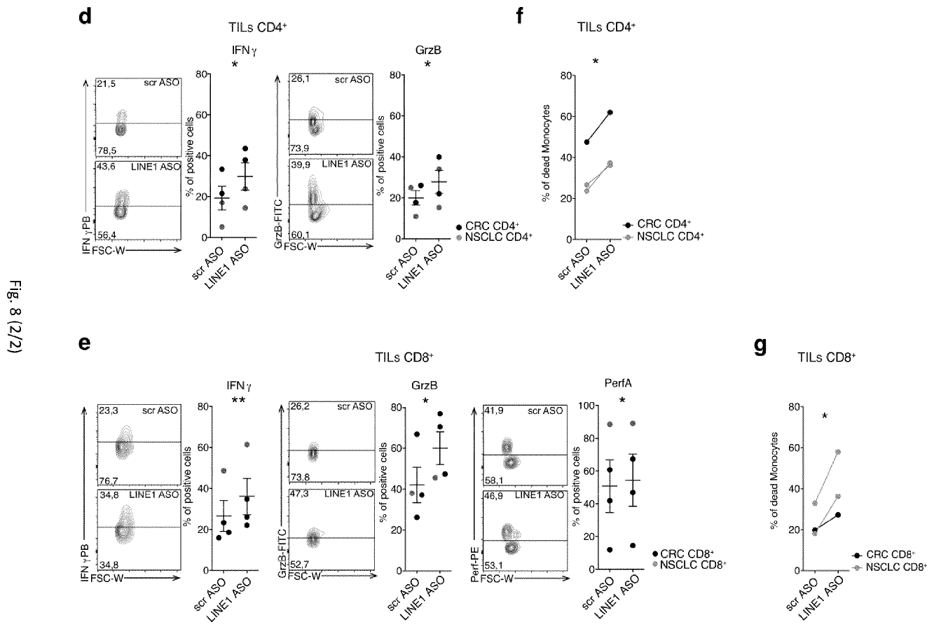

FIG. 9. LINE1 RNAs are enriched in open chromatin regions of naïve CD4⁺ T-cells.

(a) Representative confocal fluorescence microscopy images of Alu RNA FISH (red) performed on quiescent naïve and memory CD4⁺ and CD8⁺ T-cells. As control, naïve CD4⁺ T-cells were treated with RNAse. Original magnification 63×. Scale bar 5 μm. (b) Violin plot representation of Alu RNA FISH signal from four individuals; at least 220 nuclei per group were assessed. *** P<0.0001, Ordinary One-way ANOVA. (c) Alu expression levels by qRT-PCR in quiescent naïve and memory Th1, Th2, Th17 CD4⁺ T-cells and in quiescent naïve and memory CD8⁺ T-cells, each dot represents a different donor. (d) Representative confocal fluorescence microscopy images of HERV RNA FISH (red) performed on quiescent naïve and memory CD4⁺ and CD8⁺ T-cells. As control, naïve CD4⁺ T-cells were treated with RNAse. Original magnification 63×. Scale bar 5 μm. (e) Violin plot representation of HERV RNA FISH signal from three individuals; at least 164 nuclei per group were assessed. (f) HERV expression levels by qRT-PCR in quiescent naïve and memory Th1, Th2, Th17 CD4⁺ T-cells and in quiescent naïve and memory CD8⁺ T-cells, each dot represents a different donor. (g) Abundance of 18S and Xist (cytoplasmic, and chromatin-associated control transcripts), in the cytoplasm, nucleoplasm and chromatin of quiescent naïve CD4⁺ T-cells from three individuals. Data are represented as mean. (h) Representative confocal fluorescence microscopy images of LINE1 RNA FISH (red) and Immunofluorescent staining (gray) for H3K4me3 and H3K9me3 on quiescent naïve CD4⁺ T-cells. Original magnification 63×. Scale bar 5 μm. (i) Pearson correlation of colocalization between RNA FISH and Immunostaining has been measured in three individuals; at least 103 nuclei per group were assessed. *** P<0.0001, Ordinary One-way ANOVA. Quiescent naïve CD4⁺ T-cells were activated with TCR engagement and (j) Th2 or (k) Th17 cytokines cocktail. LINE1 expression levels by qRT-PCR at at 1, 3, 5, 7 days in four individuals for (a) and (b). * P=0.0209 Ordinary One-way ANOVA; ** P=0.0100 Ordinary One-way ANOVA. (1) LINE1 expression levels by qRT-PCRs in naïve CD4+ T-cells that were activated with TCR engagement and Th1 cytokines cocktail for 72 hours and then treated with different signaling pathway inhibitors for 48 hours (n=4 individuals). Control vs Rapamycin * P=0.0286 Two-tailed Mann-Whitney Test. (m) Phosphorylated S6 protein (pS6, mTORC1 target) levels were assessed by quantitative western blot in naïve CD4⁺ T-cells that were activated with TCR engagement and Th1 cytokines cocktail, for 72 hours and then treated with Rapamycin or CsA. 3 Tubulin is used as loading control. (n) Representative confocal fluorescence microscopy images of LINE1 RNA FISH (red) performed on quiescent naïve CD4⁺ T-cells that were activated with TCR engagement and Th1 cytokines cocktail, for 72 hours and then treated with Rapamycin or CsA. Original magnification 63×. Scale bar 10 μm.

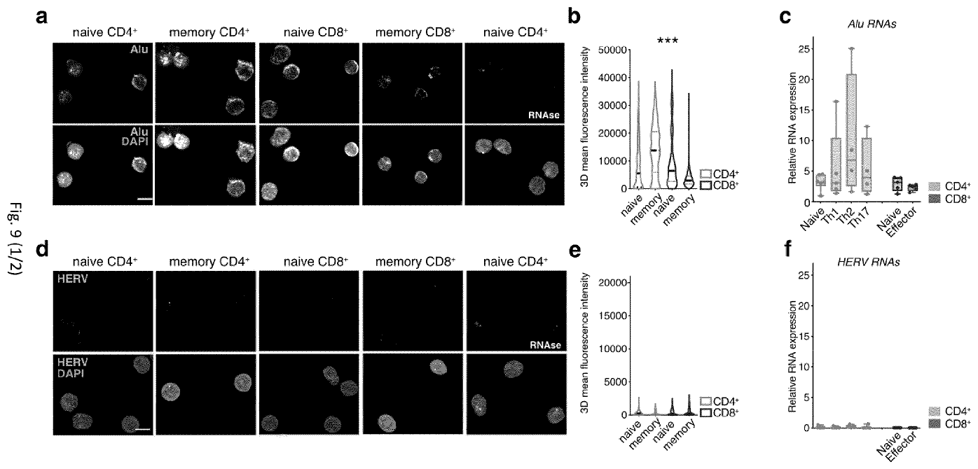

FIG. 10. Naive CD4+ T-cells express evolutionary old LINE1 elements in contrast to mESC that express evolutionary young, retrotransposition competent LINE1 elements.

(a) Heatmap of transposable elements expression at class, superfamily and subfamily level in each chromatin and nucleoplasm naïve CD4⁺ T-cells RNA-seq replicate. Z-score was computed on the log 2 transformed normalized read count using DESeq2. (b) Scatter plot of LINE1 subfamilies expressions in nucleoplasm (x-axis) and chromatin (y-axis) RNA-seq of naïve CD4⁺ T-cells. Subfamilies are color coded based on evolutive origin: mammalian-specific (LIM, orange), primate-specific (LIP, blue), human-specific (L1Hs, green), HAL (yellow). (c) Heatmap of transposable elements expression at class, superfamily and subfamily level in each mESCs RNA-seq replicate. Z-score was computed on the log 2 transformed normalized read count using DESeq2 (d and f) Pie-chart representing distribution of chimeric and pure reads for Hs LINE1 (d) and Mm LINE1 (f) reported as average percentage across the replicates (see methods) in naïve CD4⁺ T-cells. (e and g) Hs LINE1 (e) and Mm LINE1 (g) transcripts genomic distribution among protein coding, intergenic regions, lncRNAs, pseudogenes and ncRNAs transcriptional units in naïve CD4⁺ T cells

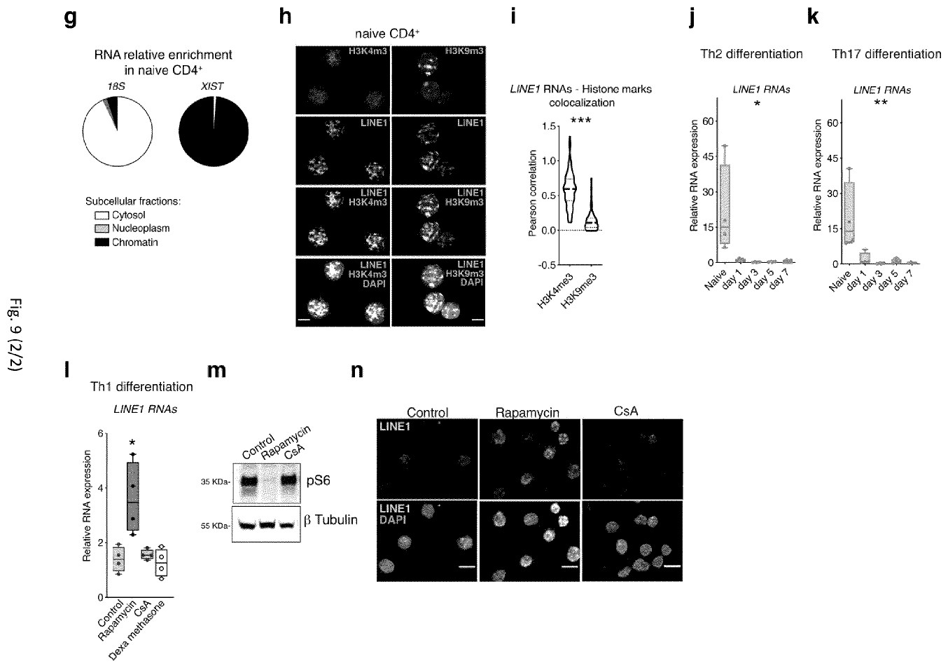

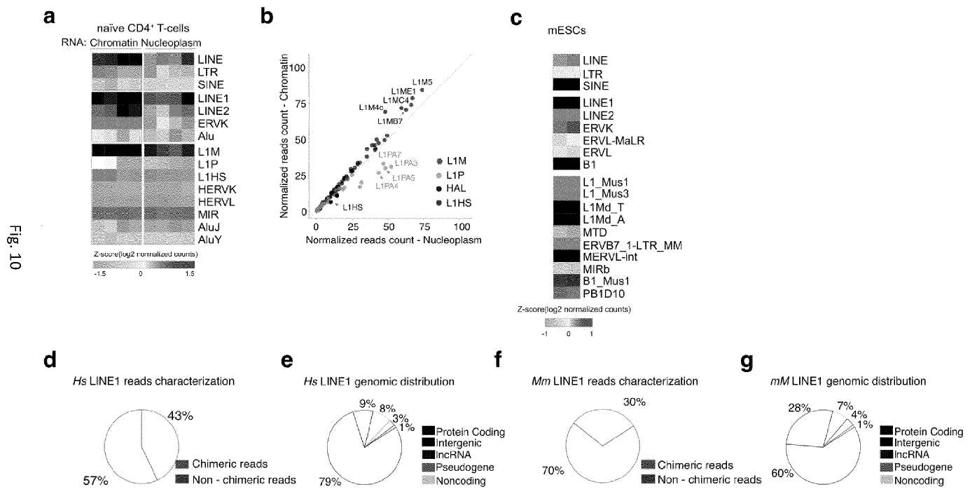

FIG. 11. Validations of the novel LINE1 transcripts.

(a) Long-read transcriptional profile of LINE1 transcripts detected by Nanopore sequencing (n=407). The line and area represent respectively the mean coverage and the standard error of the mean. (b-i) Scheme of the LINE1 containing genes mRNAs and of the novel reconstructed LINE1 transcripts; the LINE1 exon is represented in orange. In the middle, schemes of the PCR primers designed to verify the presence of the two transcriptional isoforms are reported. Below, agarose gel for the PCR results for LINE1 transcript, LINE1 containing gene mRNAs and negative control.

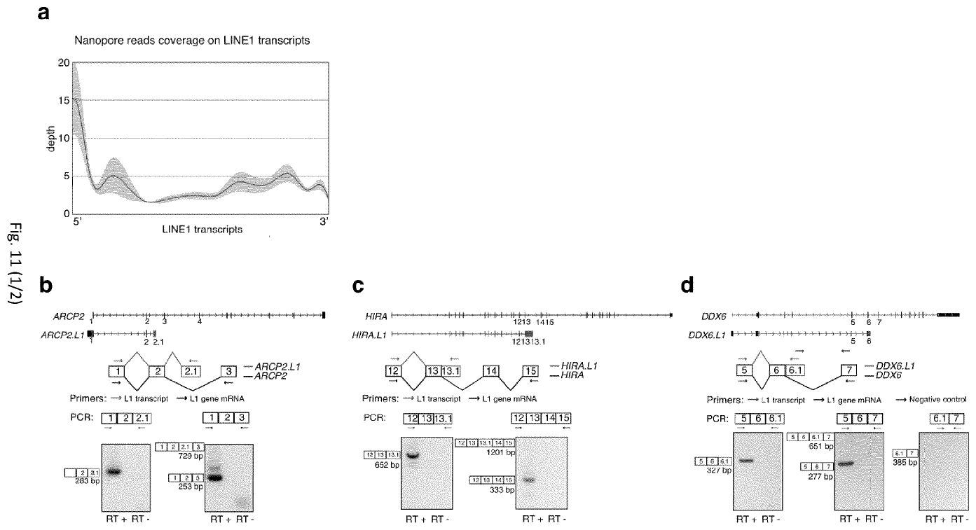

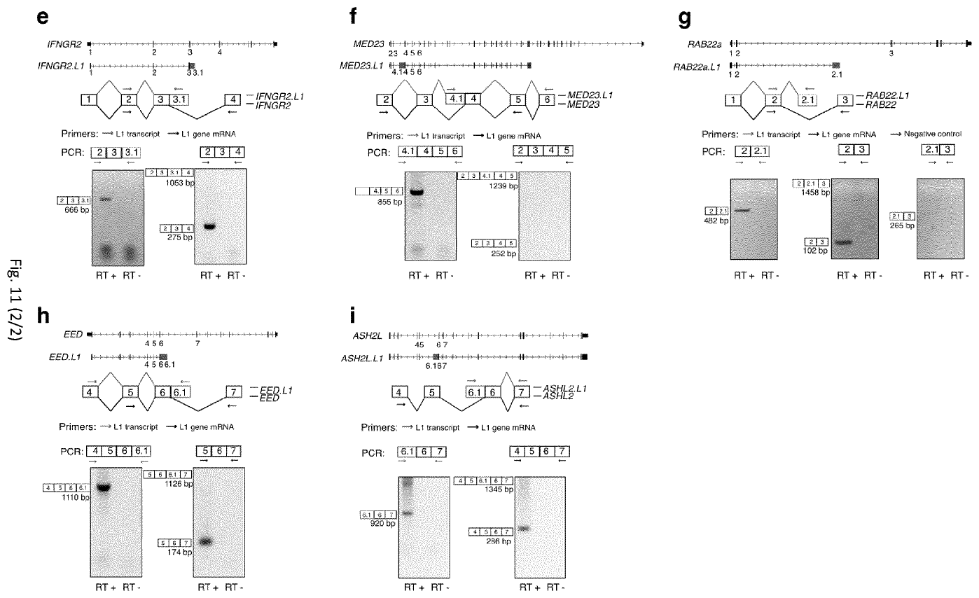

FIG. 12. Evolutionary old and intronic LINE1 elements (retrotransposition incompetent) are spliced as novel exon of non-canonical splicing variants of genes important for cellular activation.

(a) Length distribution of the LINE1 loci. The dashed line shows the average length of the LINE1 loci. (b) LINE1 loci position distribution in respect to a full length LINE1 sequence (6 kb). Primers used for qRT-PCR, probes for RNA FISH and antisense oligonucleotide (ASOs) for LINE1 knock down experiments are shown. Right, percentage of LINE1 loci within ORF1, ORF2, 5′UTR and 3′UTR of the full length LINE1 sequence. (c) Bar plot showing the percentage of the most enriched LINE1 subfamilies in the LINE1 transcripts. (d) LINE1 loci distribution among introns, exons, promoters, 5′UTR and 3′UTR of LINE1 containing protein coding genes. (e) Consensus motifs of the donor and acceptor splicing sites of the LINE1 exon. (f) LINE1 RNA FISH performed in naïve CD4+ T-cells treated with vehicle (DMSO) or 3TC retrotranscriptase inhibitor.

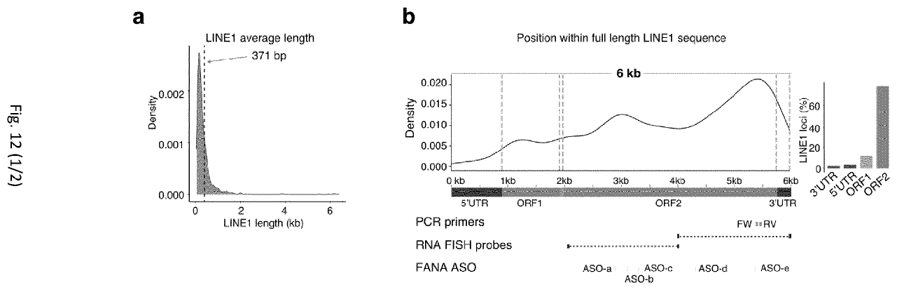

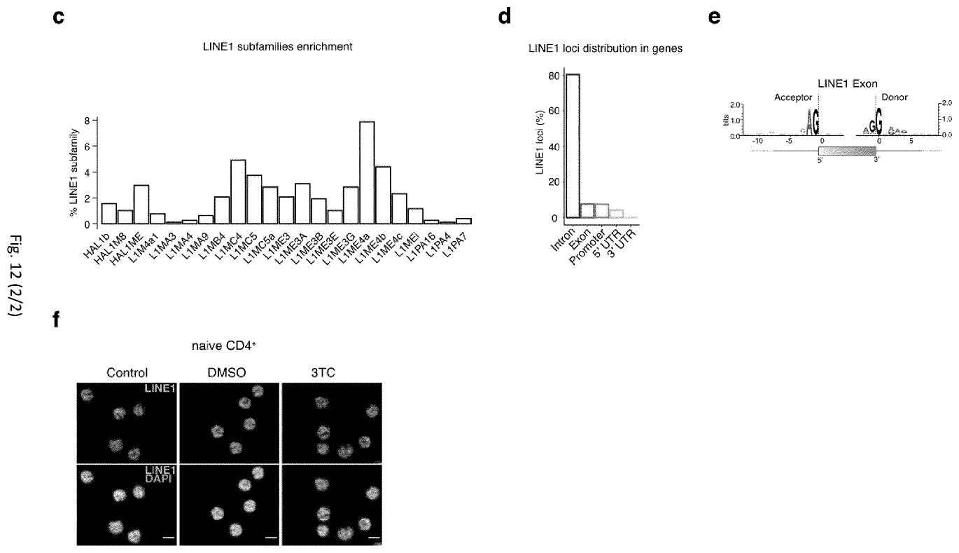

FIG. 13. LINE1 transcripts levels in quiescent naïve CD4⁺ T-cells keep paused in cis the expression of the canonical transcripts.

(a) Left, representative widefield fluorescence microscopy images of smRNA FISH for RAB22A.L1 performed on quiescent and activated naïve CD4⁺ T-cells and on naïve CD4⁺ T-cells that have been treated for 48 hours with RAB22A.L1 ASOs or control (Scr) ASOs. Original magnification 100×. Scale bar 5 μm. (b) Bar plot representing number of dots per nuclei. (c) Representative widefield fluorescence microscopy images of TSA RNA FISH on RAB22A.L1 (red) combined with DNA FISH for RAB22A genomic locus (green) performed on quiescent naïve CD4⁺ T-cells. Original magnification 100×. Scale bar 5 μm. (d) RAB22A.L1 and Canonical transcripts expression levels by qRT-PCR in quiescent naïve CD4⁺ T-cells that have been treated for 48 hours with RAB22A.L1 or control (Scr) ASOs (n=3 individuals). * P=0.04, Two-tailed paired t test. (e) Schematic representation of ARPC2 LINE1 transcripts deletion with Cas9/RNP in quiescent naïve CD4⁺ T cells. Naïve CD4⁺ T cells were nucleofected with Cas9/RNP and kept in culture for 96 hours. (f) ARPC2.L1 and Canonical transcripts expression level by qRT-PCRs in quiescent naïve CD4⁺ T cells 96 hours after nucleofection with Cas9/RNP for ARPC22.L1 or control (n=4 individuals). LINE1 transcripts, ARCP2.L1 * P=0.04 One-tailed paired t test (g) Up, schematic representation of sgRNAs (blue) and control PCR primers (black) position in IFNGR2 locus. Middle, PCR validation on 25 ng of gDNA extracted from naïve CD4⁺ T-cells 96 hours after nucleofection with Cas9/RNP targeting IFNGR2.L1 or control (n=4 individuals). The primers used are designed outer sgRNAs sequence. Bottom, schematic representation of predicted deletion loci (sgRNA in blue, PAM in red) with sanger sequencing analysis of PCR validation. (h) Up, schematic representation of sgRNAs (blue) and control PCR primers (black) position in ARCP2 locus. Middle, PCR validation on 25 ng of gDNA extracted from naïve CD4⁺ T-cells 96 hours after nucleofection with Cas9/RNP targeting ARCP2.L1 or control (n=3 individuals). The primers used are designed outer sgRNAs sequence. Bottom, schematic representation of predicted deletion loci (sgRNA in blue, PAM in red) with sanger sequencing analysis of PCR validation.

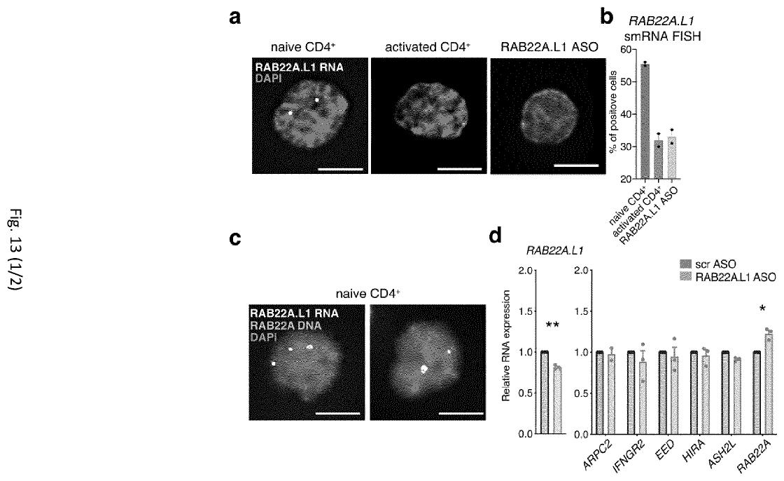

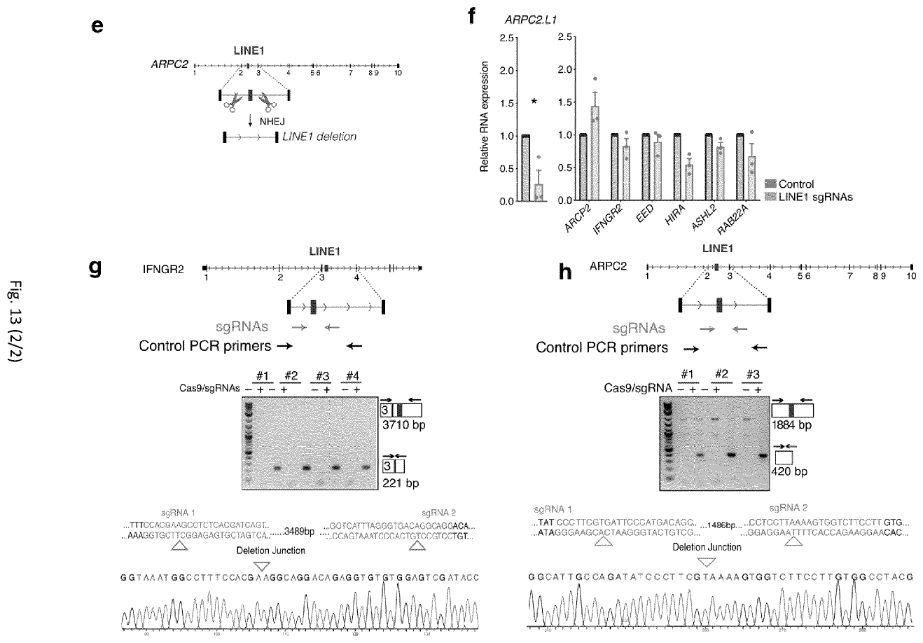

FIG. 14. LINE1 transcripts hamper H3K36me3 deposition on the LINE1 containing genes.

(a) LINE1 expression levels by qRT-PCRs in quiescent naïve CD4⁺ T-cells that have been treated for 48 hours with LINE1 or control (Scr) ASOs (n=8 individuals). Data are represented as mean and ±s.e.m. *** P<0.0001 Two-tailed paired t test. (b) Representative confocal fluorescence microscopy images of LINE1 RNA FISH (red) and Immunofluorescent staining (gray) for H3K36me3 and H3K4me3 performed on quiescent naïve CD4+ T-cells that have been treated for 48 hours with LINE1 or control (Scr) ASOs. Original magnification 63×. Scale bar 5 μm. (c) Violin plot representation of H3K36me3 and H3K4me3 signal from naïve CD4+ T-cells treated with LINE1 or control (Scr) ASOs isolated from two individuals; at least 267 nuclei per group were assessed. *** P<0.001 Two-tailed Mann-Whitney Test. (d) Positional distribution of H3K4me3 ChIP seq signal plotted on LINE1 containing genes or control genes bodies compared between naïve and activated CD4+ T-cells. The median of H3K34me3 signal (ChIP/Input fold enrichment) across the genes is plotted. (e-f) Representative ChIP-seq tracks of H3K4me3 and H3K36me3 for ERGIC2 LINE1 containing genes and FUCA2 control genes in quiescent naïve and naïve CD4+ T-cells activated for 16 hours with TCR engagement and Th1 cytokines cocktail. LINE1 transcripts and LINE1 genomic positions are represented. ChIP-seq coverage tracks are normalized to their respective input. (g) Canonical transcripts and control gene (HECW1) were amplified by qRT-PCRs in H3K36me3 ChTP experiments performed in quiescent naïve and naïve CD4+ T-cells activated for 16 hours with TCR engagement and Th1 cytokines cocktail (n=3 individuals). Data are represented as mean % of input±s.e.m. H3K36me3 ChTP naïve CD4+ T-cells vs activated CD4+ T-cells*** P<0.0001, F=69.42 Two-way ANOVA. (h) Canonical transcripts and control gene (HECW1) were amplified by qRT-PCRs in H3K36me3 ChIP experiments performed in quiescent naïve CD4+ T-cells that have been treated for 48 hours with LINE1 or control (Scr) ASOs (n=3 individuals). Data are represented as mean % of input±s.e.m. H3K36me3 ChIP Scr vs LINE1 *** P<0.0001, F=58.86, Two-way ANOVA.

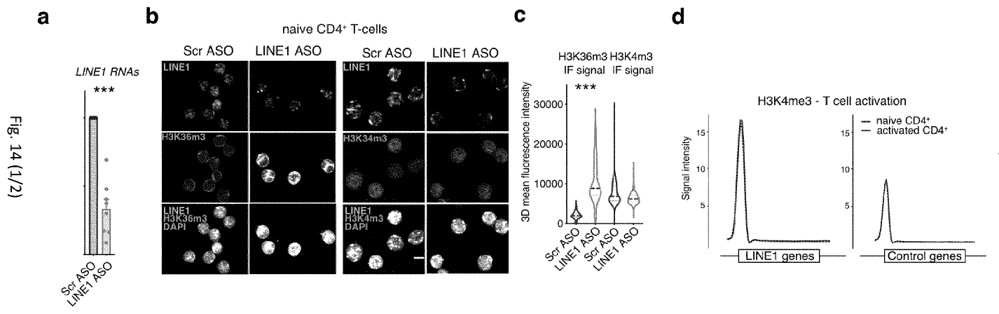

FIG. 15. LINE1 transcripts in partnership with Nucleolin interfere with the transcription of the LINE1 containing genes.

(a) Nucleolin expression levels by qRT-PCRs and (b) protein levels in quiescent naïve CD4⁺ T-cells that have been treated for 48 hours with Nucleolin or control (Scr) ASOs, four individuals were analyzed. Data are represented as mean and ±s.e.m. * P=0.0482 Two-tailed paired t test. (c) Representative confocal fluorescence microscopy images of LINE1 RNA FISH (red) and Immunofluorescent staining (gray) for H3K36me3 performed on quiescent naïve CD4⁺ T-cells that have been treated for 48 hours with Nucleolin or control (Scr) ASOs. Original magnification 63×. Scale bar 5 μm. (d) Violin plot representation of H3K36me3 signal from naïve CD4⁺ T-cells treated with Nucleolin or control (Scr) ASOs isolated from three individuals; at least 259 nuclei per group were assessed. *** P<0.001, Two-tailed Mann-Whitney Test. (e) Abundance of GAPDH and MALAT1 (cytoplasmic, and nuclear control transcripts) in the cytoplasm, nucleoplasm and chromatin of quiescent naïve CD4⁺ T-cells that have been treated for 48 hours with Nucleolin or control (Scr) ASOs (n=3 individuals). Data are represented as mean.

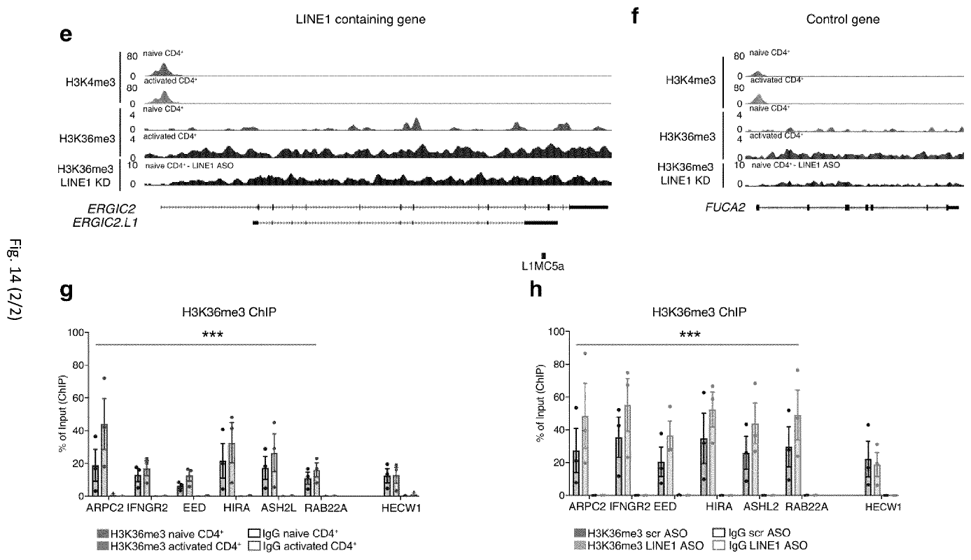

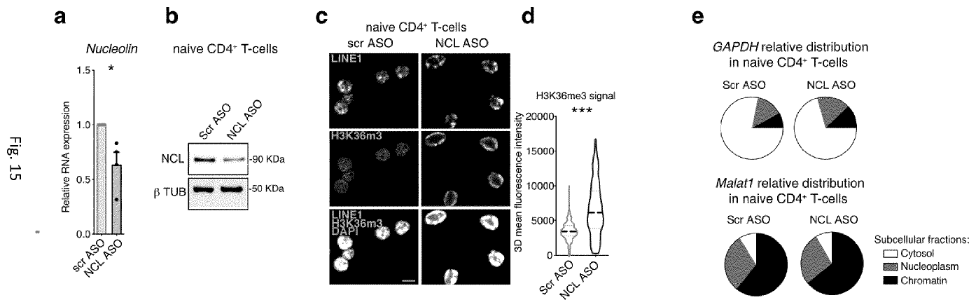

FIG. 16. LINE1 transcripts are downregulated upon T-cell activation while canonical transcripts are upregulated.

(a) Expression level of the Canonical transcripts and three random control set of control genes that do not retain genomic LINE1 elements (control genes no LINE1) and control genes that retain LINE1 elements but do not generate LINE1 transcripts (control genes with LINE1) in RNA-seq datasets of quiescent and activated naïve CD4+ T-cells. *** P<0.001 Wilcoxon matched-pairs signed rank test. (b) LINE1 transcripts and Canonical transcripts expression levels by qRT-PCRs in quiescent naïve and activated CD4+ T-cells for 16 hours with TCR engagement and Th1 cytokines cocktail (n=3 individuals). Data are represented as mean and ±s.e.m. LINE1 transcripts ** P=0.0024, F=13.65 Two-way ANOVA; Canonical transcripts *** P=0.0003, F=23.30 Two-way ANOVA. (c) Transcription factors (TFs) binding motif research was performed on the promoter regions of the LINE1 containing genes; TFs statistically upregulated in naïve CD4+ T-cells in respect to naïve CD8+ T-cells both in RNA-seq and proteomics datasets (see methods) were filtered, IRF4 is one of the most enriched. (d) IRF4 expression levels by qRT-PCRs in quiescent naïve and activated CD4+ T-cells (n=3 individuals). Data are represented as mean and ±s.e.m. * (e) IRF4 expression levels by qRT-PCRs in quiescent naïve CD4+ T-cells that have been treated for 48 hours with IRF4 or control (Scr) ASOs (n=3 individuals). Data are represented as mean and ±s.e.m. ** P=0.0042, Two-tailed paired t test. (f) IRF4 levels by FACS analysis in quiescent naïve CD4+ T-cells that have been treated for 48 hours with IRF4 or control (Scr) ASOs.

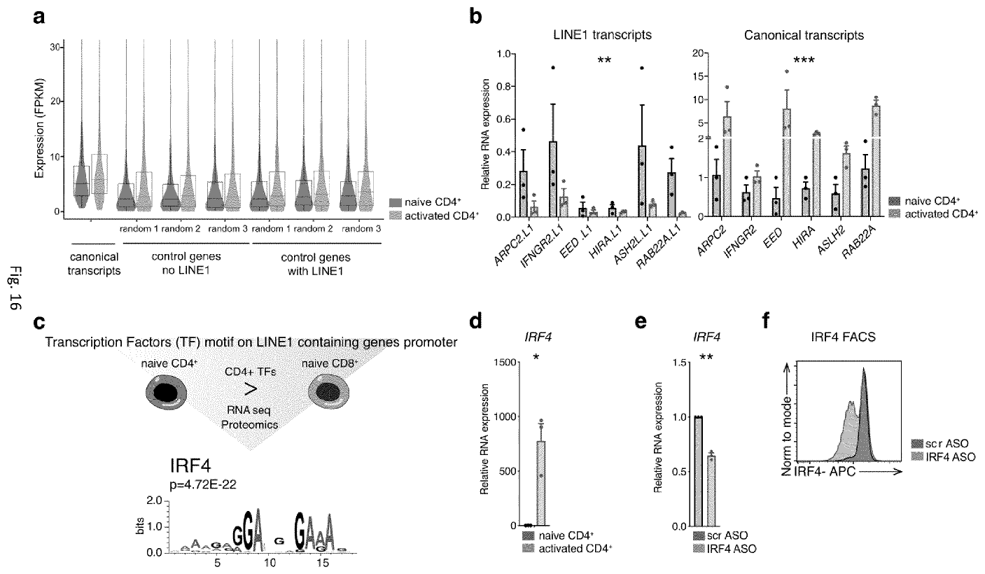

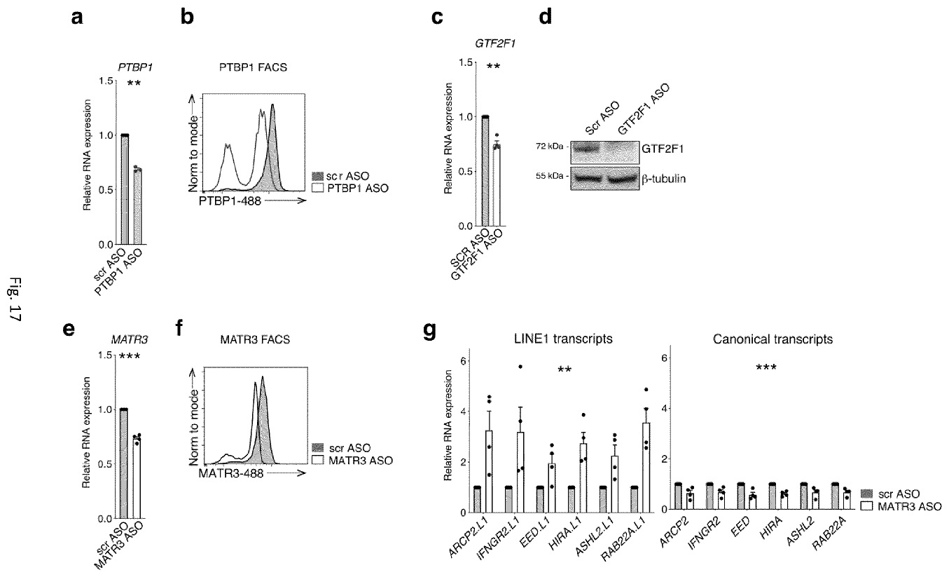

FIG. 17. MATR3/PTBP1 suppress LINE1 exons splicing in activated CD4+ T-cells.

(a) PTBP1 expression levels by qRT-PCRs in quiescent naïve CD4+ T-cells that have been treated for 48 hours with PTBP1 or control (Scr) ASOs and then activated for 16 hours via TCR engagement in the presence of Th1 cytokine cocktail. Data are represented as mean and D s.e.m. n=3 individuals **P=0.0014 Two-tailed paired t test. (b) PTBP1 levels by FACS analysis in activated CD4+ T-cells treated with PTBP1 or control (Scr) ASOs. (c) GTF2F1 expression levels by qRT-PCRs and protein levels (d) in quiescent naïve CD4+ T-cells that have been treated for 48 hours with GTF2F1 or control (Scr) ASOs, four individuals were analyzed. Data are represented as mean and ±s.e.m. ** P=0.0031, Two-tailed paired t test. (e) MATR3 expression levels by qRT-PCRs in quiescent naïve CD4+ T-cells that have been treated for 48 hours with MATR3 or control (Scr) ASOs and then activated for 16 hours via TCR engagement in the presence of Th1 cytokine cocktail. Data are represented as mean and ±s.e.m. n=3 individuals *** P=0.0006 Two-tailed paired t test. (f) MATR3 levels by FACS analysis in activated CD4+ T-cells treated with MATR3 or control (Scr) ASOs. (g) LINE1 transcripts and Canonical transcripts expression levels by qRT-PCRs in activated CD4+ T-cells that have been treated for 48 hours with MATR3 or control (Scr) ASOs and then activated for 16 hours via TCR engagement in the presence of Th1 cytokine cocktail (n=4 individuals). LINE1 transcripts, *** P<0.001, F=44.8, Two-way ANOVA; Canonical transcripts, ** P=0.006, F=29.3, Two-way ANOVA.

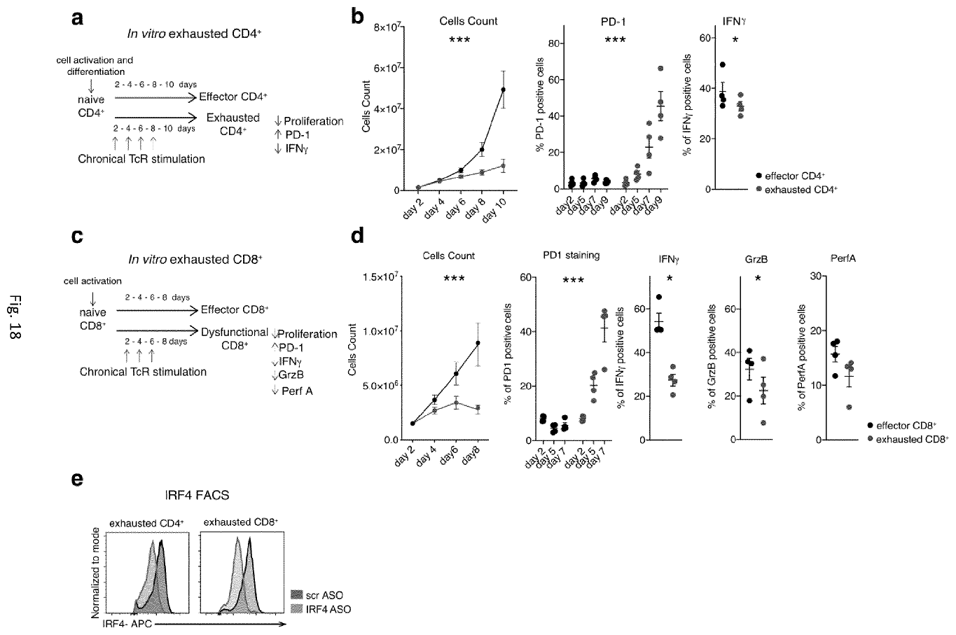

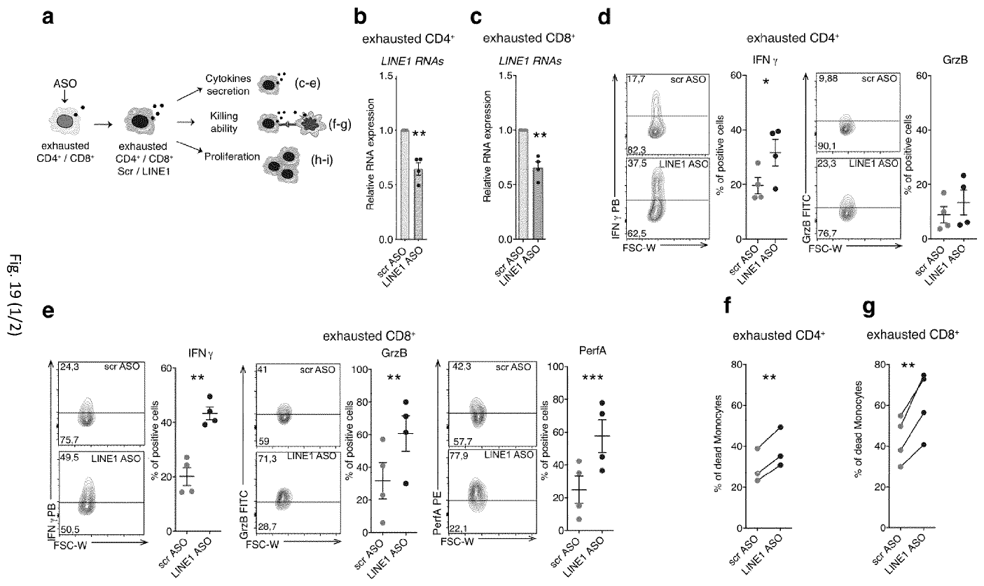

FIG. 18. LINE1 RNAs re-accumulate in dysfunctional CD4⁺ and CD8⁺ T lymphocytes in vitro.

(a) Quiescent naïve CD4⁺ T-cells were activated and differentiated to Th1 and exposed every 2 days to a stimulatory anti-CD3 mAb; chronical anti-CD3 stimulation induces growth arrest, PD-1 surface marker expression and reduction of IFNγ production. (b) On the left, cell count of effector CD4⁺ and dysfunctional CD4⁺ T-cells at 2-4-6-8-10 days (n=5 individuals). Data are represented as mean and s.e.m, *** P<0.0001, F=57.22 Two-way ANOVA. In the middle, PD-1 positive cells in effector CD4⁺ and dysfunctional CD4⁺ T-cells at 2-5-7-9 days (n=4 individuals). Data are represented as mean and ±s.e.m, *** P<0.0001, F=48.77 Two-way ANOVA. On the right, IFNγ positive cells in effector CD4⁺ and dysfunctional CD4⁺ T-cells at 9 days (n=4 individuals). Data are represented as mean and ±s.e.m. * P=0.032. One-tailed paired t test. (c) Quiescent naïve CD8⁺ T-cells were activated and exposed every 2 days to a stimulatory anti-CD3 mAb; chronical anti-CD3 stimulation induces growth arrest, PD-1 surface marker expression and reduction of IFNγ, GrzB and PerfA production. (d) On the left, cell count of effector CD8⁺ and dysfunctional CD8⁺ T-cells at 2-4-6-8 days. Data are mean and s.e.m, N=4 individuals. *** P=0.0003, F=26.05 Two-way ANOVA. In the middle, PD-1 positive cells in effector CD8⁺ and dysfunctional CD8⁺ T-cells at 2-5-7 days. Data are mean and s.e.m, N=4 individuals. *** P<0.0001, F=58 Two-way ANOVA. On the right, IFNγ, GrzB and PerfA positive cells in effector CD8⁺ and dysfunctional CD8⁺ T-cells at 9 days. Data are mean and +s.e.m, N=4 individuals. IFNγ * P=0.01; GrzB * P=0.02. Two-tailed Paired t test. (e) IRF4 levels by FACS analysis dysfunctional CD4⁺ and CD8⁺ T cells that have been treated for 48 hours with IRF4 or control (Scr) ASOs.

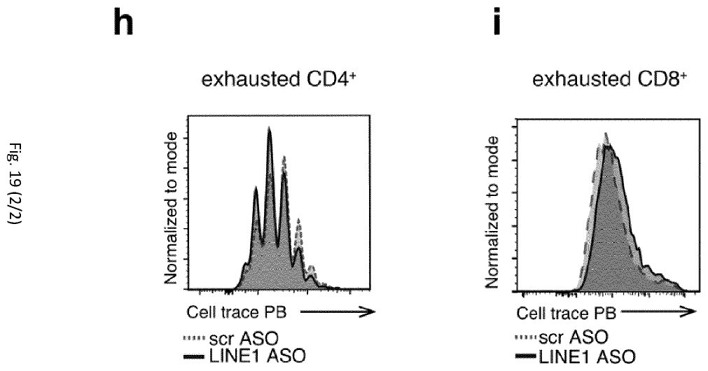

FIG. 19. LINE1 transcripts regulate the exhausted phenotype of CD4⁺ and CD8⁺ T lymphocytes in vitro.

(a) Schematic representation of the immunological assays performed on effector CD4⁺ and CD8⁺ T-cells rendered exhausted in vitro and treated with LINE1 or control (Scr) ASO. After ASO treatment, exhausted CD4⁺ and CD8⁺ T-cells will be tested for effector cytokines secretion (panel d and e), the ability to kill heterologous antigen presenting cells with MHCII and MHCI as monocytes (panel f and g) and the proliferation capacity (panel h and i) (b-c) LINE1 expression levels by qRT-PCRs in exhausted CD4⁺ (b) and CD8⁺ (c) T-cells that have been treated with LINE1 or control (Scr) ASOs (n=4 individuals). Data are represented as mean and ±s.e.m. CD4+ T-cells ** P=0.004; CD8⁺ T-cells ** P=0.007 Two-tailed paired t test. (d) Percentage of IFNγ or GrzB positive exhausted CD4⁺ T-cells that have been treated with LINE1 or control (Scr) ASOs (n=4 individuals). Data are represented as mean and ±s.e.m. * P=0.0336 One-tail paired t test. (e) Percentage of IFNγ, GrzB or PerfA positive exhausted CD8⁺ T-cells that have been treated with LINE1 or control (Scr) ASOs (n=4 individuals). Data are represented as mean and ±s.e.m. IFNγ ** P=0.002; GrzB ** P=0.004, PerfA *** P<0.001, Two-tail paired t test. (f-g) Percentages of dead heterologous Monocytes co-cultured for 12 hours with exhausted CD4⁺ (f) or CD8⁺ (g) T-cells that have been treated with LINE1 or control (Scr) ASOs. CD4⁺ T-cells ** P=0.009, CD8⁺ T-cells ** P=0.008 Two-tailed paired t test; (h-i) Proliferation assay with cell trace in exhausted CD4⁺ (h) or CD8⁺ (i) T-cells that have been treated with LINE1 or control (Scr) ASOs.

### EXAMPLE 1

Materials and Methods

Human Blood and Tissue Samples

Blood from anonymous healthy donors was provided by Fondazione Istituto di Ricovero e Cura a Carattere Scientifico (IRCCS) Ca Granda Ospedale Maggiore Policlinico in Milan. The age and the sex of healthy donors were unknown (privacy). Peripheral blood from Lymphangioleiomyomatosis (LAM) patients were obtained from Ospedale San Giuseppe-MultiMedica IRCCS in Milan. Peripheral blood from kidney transplanted patients treated with Everolimus were obtained from Fondazione IRCCS Cà Granda-Ospedale Maggiore Policlinico, Milan. Colorectal cancer (CRC) samples and Non-Small-Cell Lung cancer (NSCLC) samples were provided by European Institute of Oncology (IEO), non tumoral samples were obtained from normal adjacent tissue distal at least 10 cm from the lesion; no patients received palliative surgery or neo-adjuvant chemo- and/or radiotherapy. The ethics committees of the hospitals approved the use of human samples for research purposes and informed consent was obtained from all subjects.

T-Cells Purification and Sorting and Monocytes Purification

Human peripheral blood mononuclear cells (PBMCs) were purified from human blood samples by density gradient centrifugation with Ficoll-Paque Plus. From PBMCs, T-cells were negatively selected with magnetic separator (AutoMACS Pro Separator; Miltenyi Biotec) using Pan T cell Isolation Kit (Miltenyi Biotec) or CD4⁺ T cell Isolation Kit (Miltenyi Biotec). T-cells were stained with antibodies for surface markers and T-cells subsets were sorted by flow cytometry: naïve CD4⁺ as CD4⁺ CD25⁻CD127^{-/high}CD45RO⁻, CD4⁺ Th1 cells as CD4⁺CD25⁻CD127^{-/high}CD45RO⁺CXCR3⁺CCR6⁻, CD4⁺ Th2 cells as CD4⁺CD25⁻CD127^{-/high}CD45RO⁺CRTH2 and CD4⁺ Th17 cells as CD4⁺CD25⁻CD127^{-/high}CD45RO⁺CCR6⁺CXCR3⁻: naïve CD8⁺ as CD4⁻ CD8⁺CD45RO⁻ and memory CD8⁺ as CD4⁻CD8⁺CD45RO⁺. In order to isolate tissue infiltrating lymphocytes, tumor and normal adjacent tissues were washed several times and maintained overnight at 4° C. in Roswell Park Memorial Institute (RPMI) 1640 supplemented with 400 μg/mL gentamicin, 15 μg/mL amphotericin, 500 U/mL penicillin and 500 μg/mL streptomycin. Tissues were then weighted, smashed and treated with 5 mL/gr of tissue of EDTA chelation buffer (5 mM EDTA, 1 mM DTT and 67 μg/mL DNase I in HBSS) for 20 min at 37° C. Tissues were centrifuged at 500 g and room temperature (RT), washed with Hank's Balanced Salt Solution (HBSS) (Gibco) and digested with 5 mL/gr of digestion solution (1 mg/mL of Collagenase D and 67 μg/mL DNase diluted in HBSS, supplemented with antibiotics) for 3 h at 37° C. in agitation. Cells released were passed through a 70-μM strainer, washed two times with HBSS for 10 min at 500 g and 4° C. and stratified through Percoll gradient (100%-60%-40%-30%) for 30 min at 400 g. T-cells were recovered from the interface between 60% and 40% Percoll layers and were stained with antibodies for surface markers. T-cells subsets were then sorted by flow cytometry: memory CD4+as CD45⁺CD3⁺ CD4⁺CD25⁻CD127^{-/high}CD45RO⁺ and memory CD8⁺ as CD45⁺CD3⁺CD8⁺CD45RO³⁰. The following antibodies were used for flow cytometry-based sorting: anti-CD4-APCCy7 (BD Biosciences; clone: RPA-T4) or anti-CD4-VioGreen (Miltenyi Biotec; clone: VIT4); anti-CD8-VioGreen (Miltenyi Biotec; clone: REA-734) or anti-CD8-VioBlue (Miltenyi Biotec; clone: REA734); anti-CD25-PECy7 (Invitrogen by Life Technologies; clone: BC96); anti-CD127-PECy5 (BioLegend; clone: A019D5) or anti-CD127-PE (Miltenyi Biotec; clone: MB15-18C9); anti-CD45RO-BV605 (BioLegend; clone: UCHL1) or anti-CD45RO-APC (Miltenyi Biotec; clone: UCHL1); anti-CD3-PE (BD Biosciences; clone:UCHT1); anti-CD45-Pacific Blue (BioLegend; clone 2D1); anti-CD183-PECy5 (BD Biosciences; clone: 1C6/CXCR3); anti-CD294(CRTH2)-APC-Vio770 (Miltenyi Biotec; clone: REA598); anti-CCR6-FITC (BioLegend; clone: G034E3). Cell sorting was performed using FACSAria III (BD Bioscience). The purity of sorted cells was >97.5%. Monocytes were isolated from PBMCs by positive selection with magnetic separator (AutoMACS Pro Separator; Miltenyi Biotec) using CD14 Microbeads (Miltenyi Biotec).

CD4⁺ and CD8⁺ T-Cells In Vitro Differentiation

Quiescent naïve CD4⁺ T-cells have been plated at 1,5×10⁶/mL, stimulated with Dynabeads Human T-activator anti-CD3/anti-CD28 beads (Gibco; cat. num. 1131D) and cultured for hours (activated naïve CD4+ T-cells) or days (effector CD4+ T-cells) with the appropriate T helper medium of differentiation. T helper medium of differentiation consists in complete medium composed by RPMI 1640 with GlutaMAX-I (Gibco) supplemented with 10% (v/v) Fetal Bovine Serum (FBS) (Gibco), 1% (v/v) non-essential amino acids, 1 mM sodium pyruvate, 50 U/mL penicillin, 50 μg/mL streptomycin, plus T helper specific cytokines. Th1 cytokines: 20 IU/mL recombinant IL-2 (cat. num. 130-097-744), 10 ng/mL recombinant IL-12 (cat. num. 130-0976-704), 2 μg/mL neutralizing anti-IL-4 (cat. num. 130-095-753). Th2 cytokines: 100 IU/mL recombinant IL-2, 10 ng/mL recombinant IL-4 (cat. num. 130-093-919), 2 μg/mL neutralizing anti-IL-12 (cat. num. 130-095-755) and anti-IFN-γ (cat. num. 130-095-743). Th17 cytokines: 10 ng/mL recombinant IL-1b (cat. num. 130-095-374), 10 ng/mL IL-6 (cat. num. 130-095-365) and 10 ng/mL IL-23 (cat. num. 130-095-757), 1 ng/mL recombinant TGF-β1 (cat. num. 130-108-971), 2 μg/mL neutralizing anti-IL-12, anti-IL-4 anti-IFN-γ (Miltenyi Biotec). Quiescent naïve CD8⁺ T cells were plated at 1,5×10⁶/mL, stimulated with Dynabeads Human T-activator anti-CD3/anti-CD28 beads and cultured for days (effector CD8+ T cells) in complete medium supplemented with 20 IU/mL recombinant IL-2. Cells were maintained at 37° C. in a 5% CO₂ humidified incubator, were counted and split every 2-3 days.

In Vitro Dysfunctional CD4⁺ and CD8⁺ T-Cells

Dysfunctional chronically stimulated CD4⁺ and CD8⁺ T-cells were generated as described in⁶⁶, with minor modifications. Briefly, naïve CD4⁺ T-cells were activated and differentiate to Th1 phenotype while naïve CD8⁺ T-cells were activated with complete medium supplemented with 20 IU/mL recombinant IL-2, every 2 days T-cells were counted and exposed to stimulatory anti-CD3/anti-CD28 beads. Dysfunctional chronically stimulated T-cells were tested for proliferation reduction, for PD-1 marker increase and for T-cell effector properties assessed with intracellular staining for lineage specific cytokines. Immunosuppressed CD4⁺ T-cells were generated as described in⁶⁷, briefly naïve CD4⁺ T-cells activated to differentiate to Th1 for 4 days were cultured with 50 ng/mL TGF-β for 24-72 hours. Effector properties of immunosuppressed T-cells were tested with intracellular staining for lineage specific cytokines.

T-Cells Treatments

Quiescent naïve CD4⁺ T-cells were treated for 16 h with Actinomycin D 5 μg/mL (Merck; cat. num. A9415) as reported in⁶⁸. TCR signaling pathway inhibition was performed using the following immunosuppressive drugs: Rapamycin (100 nM; Merck; cat. num. R8781) for mTORC1, Ciclosporin A (0,5 μg/mL; Merck; cat. num. C3662) for Calcineurin pathway, Dexamethasone (1 μM; Merck; cat. num. D4902) for NF-κB pathway. Quiescent naïve CD4⁺ T-cells were pretreated for 2 hours with the overmentioned drugs, then stimulated with Dynabeads Human T-activator anti-CD3/anti-CD28 beads in Th1 medium, in the presence of the different inhibitors; T-cells were collected 2, 4, 8 h post activation. Otherwise, naïve CD4⁺ T-cells activated and cultured in Th1 medium for 72 h were treated for 48 h with the drugs. Treated cells were controlled for their vitality and treatment efficacy was assessed.

Knock Down Experiments

Knock down experiments have been performed using FANA (2′-deoxy-2′-fluoro-β-D-arabinonucleid acid, https://www.aumbiotech.com)—antisense oligonucleotides (ASOs). For GTF2F1, Nucleolin, MATR3 and PTBP1 mRNA four ASOs were used; for IRF4 mRNA two FANA-ASOs were used. For LINE1 RNAs five ASOs were designed on ORF2 region of LINE1 consensus sequence while for HIRA.L1 or RAB22A.L1 three ASOs were designed on a unique and specific sequence portion of the LINE1 transcripts. An unrelated scramble (Scr) ASO was used as control. ASOs were mixed in equimolar proportion and administered without any transfection reagent (by gymnosis) following manufacturer's instruction at a final concentration of 10 μM. Quiescent naïve CD4⁺ T-cells isolated from healthy donors were cultured for 48 h in complete medium supplemented with 200 IU/mL recombinant IL-2 and 10 μM ASOs; naïve CD4+ treated with ASOs were activated with anti-CD3/anti-CD28 beads in Th1 medium and in presence of 10 μM ASOs, T-cells were collected after 16 h (activated CD4⁺) or seven days (effector CD4⁺). Chronically stimulated CD4⁺ T-cells were treated starting from day 2 with 10 μM ASOs and collected for subsequent analysis at day 9 for LINE1 knock down, otherwise they were treated from day 6 for 48 h for IRF4 knock down. Memory CD4⁺ and CD8⁺ T-cells isolated from tumor samples were cultured for 48 h in complete medium supplemented with 200 IU/mL recombinant IL-2 and 10 μM ASOs. After 48 h of ASOs treatment the cells where subjected to surface marker staining and T-cells killing alternatively cells were activated for additional 48 h and subjected to intracellular cytokines staining. Knock down efficiency was controlled by RT-qPCR and/or RNA-FISH and by western blot or FACS analysis (described below).

T-Cells Surface and Intracellular Staining and Proliferation Assay

Surface markers' staining was performed incubating 1 μL of antibody for 5×10⁴ cells in phosphate-buffered saline (PBS) at 37° C. for 30 min. T-cells were washed in PBS and then analyzed. The following antibodies were used: anti-CD279 (PD-1)-Alexa Fluor 488 (BioLegend; clone: EH12.2H7), anti-CD366 (TIM3-1)-BV650 (BioLegend; clone: F38-2E2) and anti-CD223 (LAG-3)-BV785 (BioLegend; clone: 11C3C65). For intracellular cytokines and transcription factors staining 5×10⁴ T-cells were stimulated with 50 ng/mL phorbol 12-myristate 13-acetate (PMA) and with 0.5 μg/mL ionomycin for 2 h at 37° C., subsequently 100 μg/mL Brefeldin A (Merck) was added for additional 2 h at 37° C. Cells were washed, fixed and permeabilized for 30 min at 4° C. with Foxp3 Transcription Factor Fixation/Permeabilization kit (Invitrogen by Life Technologies) according to the manufacturer's instructions. Cytokines and transcription factors were stained incubating 1 μL of antibody for 5×10⁴ cells diluted in Permeabilization Buffer (Invitrogen by Life Technologies) for 20 min at RT. T-cells were washed in PBS and then analyzed. For the intracellular staining the following antibodies were used: anti-IFN-γ-V450 (clone: B27), anti-GrzB-FITC (clone: GB 11), anti-PerfA-APC (clone: deltaG9), anti-PerfA-PE (clone: deltaG9), anti-T-bet-V450 (clone: 04-46) (BD Biosciences). For MATR3, PTPB1 and IRF4 FACS staining T-cells were fixed and permeabilized, as above, for 30 min at 4° C. Then cells were incubated with 1 μL of primary antibody for 5×10⁴ cells diluted in Permeabilization Buffer (Invitrogen by Life Technologies) for 1 hour at RT. T-cells were washed in PBS and stained with secondary antibody for 30 min at RT. T-cells were washed in PBS and then analyzed. The following primary antibody were used: anti-MATR3 (Abcam cat. num. Ab151714), anti-PTBP1 (Abcam cat. num. Ab133734) and anti-IRF4 (BioLegend cat. num. 646412). As secondary antibodies were used: goat anti-rabbit-Alexa Fluor 488 (Invitrogen LifeTechnologies) and goat anti-Rat-Alexa Fluor 647 (Invitrogen LifeTechnologies). Proliferation assay in chronically stimulated cells was performed using cell trace (C34557), naïve CD4⁺ and CD8⁺ T-cells were incubated with 1 μL of cell trace for 1×10⁶ cells in phosphate-buffered saline (PBS) at 37° C. for 20 min. Cells were then washed with 10% FBS for 5 min at 37° C. and activated as reported above, proliferation was assessed seven days after activation. For all the above-mentioned analyses an average of 10⁴ cells was acquired with FACSCanto I (BD Biosciences) and data were analyzed using FlowJo v.10 software.

Killing Assay

Dysfunctional chronically stimulated effector CD4⁺ and CD8⁺ T-cells treated with ASOs were co-cultured for 12 hours with heterologous monocytes in 1:1 ratio. CD4⁺ and CD8⁺ memory T-cells infiltrating NSCLC or CRC treated with FANA-ASO for 48 h were co-cultured for 12 hours with heterologous monocytes in 1:1 ratio. After co-culturing, cells were stained with LIVE/DEAD Fixable Green Dead Cell Stain Kit (Invitrogen by Life Technologies; cat. num. L34969) for 20 min at RT, washed in PBS and stained with CD14-APC (clone: M5E2) to recognize monocytes. Monocytes were identified as CD14 positive and their viability was assessed as % of dead Monocytes. An average of 10⁴ cells was acquired with FACSCanto I (BD Biosciences) and data were analyzed using FlowJo v.10 software.

RNA Isolation and qRT-PCR

Total RNA was isolated using RNeasy Mini Kit (QIAGEN) plus QIAshredder (QIAGEN) according to manufacturer's instruction. During the extraction DNAse with RNase-free DNase Set (QIAGEN) was performed. Total RNA was reverse transcribed using SuperScript III First-Strand Synthesis SuperMix kit (Invitrogen by Life Technologies) following manufacturer's instructions. Real-time quantitative PCR was performed on StepOnePlus Real-Time PCR System (Applied Biosystem by Life Technologies) using Power SYBR Green PCR Master mix (Applied Biosystem by Life Technologies). All gene expression data were normalized to two independent housekeeping genes (18S, GAPDH). Normalized Ct value was calculated as 2-dCT or 2-ddCt. For Actinomycin D treatment a spike-in D. Malanogaster RNA was used for normalization.

RNA-FISH and RNA FISH Plus Immunofluorescence

RNA-FISH and combo RNA-FISH-immunofluorescence was performed as in⁶⁹. Briefly, antisense biotinylated riboprobes for LINE1, AluY and HERVK were in vitro transcribed using MAXIscript T7 transcription kit (Invitrogen) and Biotin RNA labeling mix (Roche). 50-100 ng of antisense biotinylated riboprobes per experiment were used. 3% paraformaldehyde (PFA) fixed T-cells were washed with 0.05% Triton-X-100 in PBS, permeabilized with 0.5% Triton-X-100 in PBS and maintained in 20% glycerol/PBS. The cells were frozen and thawed with dry ice and deproteinized with 0.1 M HCl. T-cells were hybridized with riboprobes at 52.5° C. for 3.5 min and incubated overnight at 37° C. in water bath. Glasses were washed with 50% formamide in 2×SSC, 2×SSC, 1×SSC and 4×SSC/0.2% Tween-20. T-cells were blocked in BSA and then incubated with Streptavidin HRP (1:1000; Perkin Elmer by Akoya Biosciences) diluted in TNT/BSA (0.1 M TrisHCl pH 8, 0.150 M NaCl, 0.1% NP-40, 4% BSA in DEPC). T-cells were washed 4 times with TNT and the signal was amplified incubating TSA working solution (1:150) in 1× amplification buffer for 3 min (TSA Plus Fluorescent kit Cy3.5 (Perkin Elmer)). T-cells were washed 4 times with TNT, nuclei were counterstained with 1 μg/mL 4,6-diamidino-2-phenylindole (DAPI). Glasses were mounted in antifade prolong Diamond mounting. Where RNA-FISH was coupled with Immunofluorescence T-cells were incubated with primary antibodies for H3K4me3 1:250 (Millipore 07-473), H3K36me3 1:250 (Abcam, cat. num. 9050) and H3K9me3 1:500 (Abcam, cat. num. Ab8898) in 2% BSA/10% goat serum/0.1% Tween/PBS overnight at 4° C. Secondary antibody conjugated with Alexa Fluor 647 was used. Images were obtained with Leica TCS SP5 Confocal microscope with an HCX PL APO 63x/1.40-NA-oil-immersion objective and acquired with with a 0.3 μm Z-stacks at randomly chosen fields.

RNA FISH Signal Quantification

To quantify mean fluorescence intensity of RNA signal in 3D reconstructed nuclei, images were analyzed with NIS-Elements Software (by Nikon). In the “General Analysis” a mask on DAPI signals was generated to identify single nuclei, and then a “3D measurement” of RNA signals in every nucleus was performed. To measure the colocalization of RNA and histone mark signals was used ImageJ Software to control the Pearson Correlation through the command “Colocalization Threshold” for every nucleus.

Single Molecule RNA FISH (smRNA FISH) on LINE1 Transcripts and Relative Quantification

Single molecule RNA FISH (smRNA-FISH) were performed using HuluFISH technology. Antisense riboprobes were designed by Pixelbio on specific and unique regions of HIRA.L1 or RAB22A.L1 LINE1 transcripts, they were synthetized as directly labelled in ATTO-568 for RAB22A.L1 and ATTO-647 for HIRA.L1. Quiescent naïve, 8 h activated CD4+ T-cells or naïve CD4+ T-cells knocked down for HIRA.L1 or RAB22A.L1 transcripts were seeded on polysinated glasses and fixed in 4% PFA, washed with 135 mM glycine and keep in 70% EtOH for overnight. T-cells were then rinse in 20% glycerol for 1 h and then treat with 0.025% pepsin in 0.01N HCl for 3.5 min. T-cells were then hybridized with probes diluted 1:40 in 20% formamide/2×SSC/10% Dextran Sulphate for RAB22A.L1 and 10% formamide/2×SSC/10% Dextran Sulphate for HIRA.L1 and incubated with riboprobes overnight at 37° C. in water bath. Glasses were washed three times for 5 min in 10% formamide/2×SSC for HIRA.L1 probes, 20% formamide/2×SSC and for 5 min with 2×SSC for RAB22A.L1 probes, nuclei were counterstained with 1 μg/mL 4,6-diamidino-2-phenylindole (DAPI). Glasses were mounted in antifade prolong glass mounting media. We examined smRNA FISH on an Eclipse Ti-E (Nikon Instruments) Plan Apo X objective microscope 100× oil (Nikon). We collected 0.3 Z-stacks at randomly chosen fields, a minimum of 90 cells per individual were analyzed and number of dots per cell were count by a person blind to the experimental sampling.

TSA RNA FISH Combined with DNA FISH

TSA RNA FISH combined with DNA FISH protocol was adapted from⁷⁰ and from^{69,71} Antisense TEG-biotinylated oligonucleotide for HIRA.L1 or RAB22A.L1 were synthesized by Eurofins Genomics, same sequence of smRNA FISH probes were used. Quiescent naïve cd4+ T-cells were fixed in 4% PFA, washed with 0.05% Triton-X-100 in PBS, permeabilized with 0.5% Triton-X-100 in PBS and maintained in 20% glycerol/PBS for over-night. The cells were frozen and thawed with dry ice, deproteinized with 0.1 M HCl and with 0.025% pepsin in 0.01N HCl. T-cells were hybridized with 1-6 ng of biotin probes suspended in 20% formamide/2×SSC/10% Dextran Sulphate for RAB22A.L1 and 10% formamide/2×SSC/10% Dextran Sulphate and incubated at 37° C. in water bath overnight. Glasses were washed with 50% formamide in 2×SSC, blocked TBN/BSA (0.1 M TrisHCl pH 8, 150 mM NaCl, 4% BSA in DEPC) in BSA and then incubated with Streptavidin HRP (1:10000; Perkin Elmer by Akoya Biosciences) diluted in TNT/BSA (100 mM TrisHCl pH 8, 150 mM NaCl, 0.2% Tween-20, 4% BSA in DEPC). T-cells were washed 3 times with TNT and the signal was amplified incubating TSA working solution (1:300) in 1× amplification buffer for 5 min (TSA Plus Fluorescent kit Cy3.5 (Perkin Elmer). T-cells were washed 3 times with TNT, post-fixed with 4% PFA for 2 min and then maintained in 50% formamide/2×SSC for at least 10 hours. DNA probe for HIRA or RAB22A were prepared by nick translation of BACs (HIRA: RP11-1057H19; RAB22A: RP11-452017, BACPAC Chori) and labelled with digoxigenin-11-dUTP as reported in⁶⁹. T-cells were hybridized at 75° C. for 5 min and incubated overnight at 37° C. in water bath. Glasses were washed with 2×SSC, 0.1×SSC and rinse in 4×SSC/0.2% Tween-20. T-cells were blocked in BSA and then incubated with anti-digoxigenin-488 (1:150; Vector Laboratories DI-7488) diluted in 4×SSC/0.2% Tween-20/4% BSA. T-cells were washed 3 times in 4×SSC/0.2% Tween-20 and then nuclei were counterstained with 1 μg/mL 4,6-diamidino-2-phenylindole (DAPI). Glasses were mounted in antifade prolong glass mounting. We examined TSA RNA FISH combined with DNA FISH on an Eclipse Ti-E (Nikon Instruments) Plan Apo X objective microscope 100× oil (Nikon), with a 0.3 μm Z-stacks at randomly chosen fields.

CRISPR-Cas9 Mediated Deletion of LINE1 Elements in Quiescent Naïve CD4+ T-Cells

For LINE1 element genomic deletion we used two different sgRNAs targeting the flanking sites of the repetite element, thus we designed two sgRNAs for each target sequence, i.e. LINE1 contained in ARCP2.L1 and IFNGR2.L1. We nucleofected Cas9-sgRNA ribonucleoprotein complexes in quiescent naïve CD4+ T-cells. For each sgRNA we prepared a Cas9-sgRNA complex in a ratio 1:3 by gently mix 40 μM of Alt-R S.p. HiFi Cas9 Nuclease V3 (IDT, catalog number 1081061) and 120 μM of sgRNA (Merck), the complexes were allowed to form for 15 minutes at 37° C. Both the Cas9-sgRNA complexes were added to 1×10⁶ naïve CD4⁺ T-cells that have been resuspended in 20 μL of primary cells nucleofection solution (P3 Primary Cells 4D Nucleofector X kit S, Lonza); quiescent naïve CD4+ T-cells have been previously sorted and maintained in culture for 24 h in complete medium supplemented with 200 IU/mL recombinant IL-2. Cas9-sgRNA complexes and naïve cD4+ T-cells were transferred to Nucleofection cuvette strips (P3 Primary Cells 4D Nucleofector X kit S, Lonza, catalog number LOV4XP3032) and electroporation was performed using a 4D Nucleofector (4D Nucleofector, Lonza) with EH115 pulse program. After nucleofection, cells were resuspended in complete medium supplemented with 200 IU/mL recombinant IL-2 and kept in culture for 4 days at 37° C. in a 5% CO₂ humidified incubator. Deletion was assessed by PCR, with GoTaq G2 Flexi DNA polymerase on genomic DNA purified from nucleofected naïve CD4⁺ T-cells. Primers were designed before and after sgRNAs positions on IFNGR2.L1 and ARCP2.L1. PCR products were subjected to TA cloning and Sanger sequencing similarly to⁷².

Proteins Extraction and Western Blotting Analysis

Histones extraction protocol and subsequent western blot analysis were performed as in⁷³. Briefly, 1,5×10⁶ T-cells were lysed in cytosolic extraction buffer (10 mM HEPES KOH pH 8, 10 mM KCl, 1 mM MgCl₂, 0.1 mM EDTA, 0.1 mM DTT, 1× protease inhibitor) and nuclei were collected at 1500 g and 4° C., washed three times with equal volume of cytosolic extraction buffer and resuspended in 0.2 N HCl overnight at 4° C. on the wheel. Histone extracts were collected by centrifugation at 16300 g for 10 min at 4° C. Nuclear protein extraction was performed as in 73. Briefly 1×10⁶ T-cells were lysed in cytosolic extraction buffer (10 mM HEPES KOH pH 8, 1.5 mM MgCl2, 10 mM NaCl, 1 mM DTT, 10% Glycerol, 1× protease inhibitor), nuclei were collected at 1200 g and 4° C., washed three times with equal volume of cytosolic extraction buffer and resuspended in nuclear buffer (10 mM HEPES KOH pH 8, 1.5 mM MgCl2, 300 mM NaCl, 1 mM DTT, 0.2% NP-40, 10% Glycerol, 1× protease inhibitor) complemented with 2 mM CaCl₂) and 20U MNase and kept at 37° C. for 30 min. Total protein extract were performed as in⁶⁸. Proteins extracts were quantified with a Qubit (Invitrogen) fluorometer and used for subsequent western blotting analysis. 1-5 μg of histones' extract was used for western blot while 20-40 μg of nuclear or total extract was used for western blot. Proteins were resolved on 4-12% Bolt Bis-Tris gel (Invitrogen) and transferred by wet transfer into a nitrocellulose membrane. The membrane was blocked and incubated overnight at 4° C. with primary antibody: H3K4me3 (Millipore, cat. num. 07-473), H3K9me3 (Abcam, cat. num. Ab8898), H3K36me3 (Abcam, cat. num. Ab9050), H3K27me3 (Millipore, cat. num. 07-449), H3 (Abcam, cat. num. Ab1791), anti-rpS6 (Cell Signaling, Cat #D68F8), anti-Nucleolin (Abcam cat. num. Ab22758), anti-PTBP1 (Abcam cat. num. Ab133734), anti-KAP1 (Abcam cat. num. Ab22353), anti IRF4 (Abcam cat. num.), anti-βtubulin (Abcam cat. num. Ab6046). The membrane was treated with the appropriate secondary antibody coupled with HRP and reveled by chemiluminescence using West Dura kit (Pierce Rockford, USA). The membrane was detected with a light-sensitive CCD (charge-coupled device) camera (Las 3000) with a linear response to the emitted light. The density of the protein band was measured with ImageJ software using the command “Analyze-Gel-Select lane-Plot lane”. The results were normalized to an internal loading control (H3) and expressed in terms of fold enrichment relative to the control.

Co-Immunopreciptitation (Co-IP)

Co-IP assay was performed on nuclear extract as described in⁷³ with minor modifications. Pellets of CD4⁺ T-cells were resuspended in cytosol extraction buffer (10 mM HEPES, 5 mM MgCl₂, 0.25 mM Sucrose, 0,1% NP-40, 1× protease inhibitor) and incubated for 5 min on ice, nuclei were collected at 300 g for 10 min and resuspended in nuclear extraction lysis buffer (10 mM HEPES, 1 mM MgCl₂, 0.1 mM EDTA, 300 mM NaCl, 0,5% Triton X-100, 25% glycerol, 1× protease inhibitor). Nuclei suspension was then sonicated (BRANSON A250 with a 3.2-mm tapered microtip; one cycles of 1 min at 20% amplitude, 30% of duty cycle) and nuclear extracts were collected by centrifugation at 16300 g for 10 min at 4° C. Proteins were precleared with Dynabeads proteins A/G, quantified with a Qubit (Invitrogen) fluorometer and used for subsequent Co-IP analysis. Immunoprecipitation was performed on nuclear extract by incubating 600 μg of protein with 4 μg of antibodies anti-Nucleolin (Abcam cat. num. Ab22758) and 8 μg anti-KAP1 (Abcam cat. num. 22353) overnight, on a rotating wheel at 4° C. The immunocomplexes were recovered with magnetic Dynabeads protein A/G (Invitrogen) for 2 h on the wheel at 4° C. The beads were washed one times with 600 μL Low Salt Buffer (10 mM HEPES, 1 mM MgCl₂, 0.1 mM EDTA, 150 mM NaCl, 0,1% Triton X-100, 5% glycerol), one time with High Salt buffer (10 mM HEPES, 1 mM MgCl₂, 0.1 mM EDTA, 300 mM NaCl, 0,1% Triton X-100, 5% glycerol) and one more time with Low Salt Buffer. Samples were eluted in Elution Buffer (5% SDS, 1× Loading Buffer, 10 mM DTT) and used for western blot analysis.

RNA Immunoprecipitation (RIP)

Quiescent and 16 h activated naïve CD4⁺ T-cells were cross-linked in 1% formaldehyde. Crosslinked cells were lysed in nuclear isolation buffer (10 mM Tris-HCl pH7.5, 5 mM MgCl₂, 320 mM Sucrose, 1% Triton X-100), homogenized with dounce and kept 10 min on ice. Nuclei were centrifugated at 2500 g for 15 min and resuspended in RIP Buffer (25 mM Tris-HCl pH 7.4, 150 mM KCl, 5 mM EDTA, 0.5 mM DTT, 0.5% NP-40, 0.5% SDS, 100 U/mL RNAse inhibitor) and sheared (BRANSON A250 with a 3.2-mm tapered microtip; one cycles of 1 min at 20% amplitude, 30% of duty cycle). Nuclear extracts were collected by centrifugation at 16300 g for 10 min at 4° C. An amount of nuclear extract correspondent to 1-3×10⁶ cells were incubated with 4 μg of anti-Nucleolin (Abcam cat. num. Ab22758), and 8 μg of anti-PTBP1 (Abcam cat. num. Ab133734) and anti-GTF2F1 (Abcam cat. num. Ab28179), overnight, on a rotating wheel at 4° C. The immunocomplexes were recovered with magnetic Dynabeads protein A/G (Invitrogen) for 2 h on the wheel at 4° C. The beads were washed three times with 600 μL RIP Buffer and 1 time with PBS. Crosslinking reversion was performed by incubating immunocomplexes 2 h at 55° C. in NT2 buffer (50 mM Tris-HCl pH 7.4, 150 mM NaCl, 1 mM MgCl₂, 0.05% NP-40, 1% SDS, 1.2 mg/mL proteinase K). RNA was purified by TRI-Reagent and during the extraction DNAse with Turbo DNA-free kit was performed (Invitrogen).

Chromatin and Nucleoplasm RNA Extraction

Cellular fractionation was performed as in⁷⁴ with minor modifications. Briefly 5-10×10⁶ of quiescent naive CD4⁺ T-cells were resuspended in 60 μL of Buffer A (10 mM HEPES pH 7.5, 10 mM KCl, 10% (v/v) glycerol, 340 mM sucrose, 4 mM MgCl₂, 1 mM DTT, 1× Protease Inhibitor Cocktail (PIC)), an equal volume of Buffer A 0.2% (v/v) Triton X-100 was added and T-cells were lysed for 12 min on ice. T-cells were centrifugated at 1200 g for 5 min at 4° C., the supernatant was collected representing cytosolic RNA fraction. The nuclear pellet was washed in 120 μL of NRB Buffer (20 mM HEPES pH 7.5, 50% (v/v) glycerol, 75 mM NaCl, 1 mM DTT, 1×PIC) at 900 g for 5 min at 4° C. and resuspended in 60 μL of NRB Buffer, an equal volume of NUN Buffer was added (20 mM HEPES pH 7.5, 300 mM NaCl, 1 M Urea, 1% (v/v) NP-40, 1 mM MgCl₂, 1 mM DTT) and T-cells were lysed for 5 min on ice. The lysate was centrifugated at 1200 g, 5 min at 4° C., the supernatant was collected representing nucleoplasmatic RNA fraction. The chromatin pellet was washed in 500 μL of Buffer A at 1200 g for 5 min at 4° C. and then the pellet was resuspended in 50 μL of Buffer A representing the chromatin RNA fraction. Total, nucleoplasm and chromatin associated RNA was extracted using Maxwell RSC miRNA Tissue kit (Promega, cat. num. AS1460) following manufacturer's instructions with minor adaptation.

RNA Library Preparation and Sequencing

The RNA integrity was checked with TapeStation (High Sensitivity RNA Screentape assay) and 15-75 ng of total RNA was used to prepare libraries. RNA was ribodepleted with RiboGone-Mammalian (TaKaRa, cat. num. 634846) following manufacturer's instruction and the libraries were prepared with the SMARTer Stranded RNA-Seq kit (TaKaRa, cat. num. 634836) according to manufacturer's instructions. The libraries were sequenced as paired 100 or 150 bp on Illumina NextSeq 500. RNA-seq libraries were prepared for: i) chromatin and nucleoplasm RNA from quiescent CD4⁺ naïve T-cells (4 individuals); ii) total RNA from quiescent naïve CD4⁺ T-cells and activated naïve CD4⁺ T-cells with anti-CD3/anti-CD28 beads in Th1 medium for 16 hours (3 individuals).

Processing and Alignment of RNA-Seq Datasets

Sequenced and publicly available RNA-seq samples were processed and aligned uniformly. CD8+Naïve (GSM3591829, GSM3591834) and activated (GSM3591826, GSM3591831) T cells were retrieved from Bediaga et al., mouse embryonic stem cells from ENCODE Project Consortium (GSM2400249, GSM2400250) and thymocytes samples from Buratin et al. (GSM4222226, GSM4222227, GSM4222228, GSM4222229, GSM4222230). Notably, these datasets were accurately chosen in order to be comparable with the datasets produced in the current work, i.e total RNA extraction and library preparation, read length of the analysed libraries. Fastq files were checked for reads quality using FastQC v0.11.3. BBDuk algorithm from BBMap v38.51 was used for removing adapters from read pairs (ktrim=r k=23 mink=11 hdist=1 tpe tbo), discarding rRNA-derived reads (k=31 hdist=1) matching the deposited human ribosomal RNA sequence (NCBI accession: U13369.1) and trimming low quality bases from read pairs. Quality-passing read pairs were used for alignment using STAR v2.5.4a (--outFilterScoreMinOverLread 0.3 --outFilterMatchNminOverLread 0.3 --outFilterMatchNmin 0 --outFilterMismatchNmax 10 --winAnchorMultimapNmax 200 --outFilterMultimapNmax 200) against the hg38 assembly of the human reference genome or the mm10 assembly of the mouse reference genome using annotations from the GENCODE version 25 human or GENCODE version M21 mouse GTF file to serve as splice junctions database.

Principal Component Analysis (PCA)

PCA analysis was performed on a comprehensive set of transcribed units composed by 50,596 genes from GENCODE version 25 and 1180 repeat subfamilies from UCSC Repeat Masker on human genome (hg38). Per gene count data were generated on aligned reads using HTSeq v0.12.4 (htseq-count -s yes --nonunique all) and per repeats subfamily counts were generated as described below (see “TE subfamilies expression quantification in RNA-seq datasets” subheading). PCA was performed using DESeq2 on variance stabilizing transformation (vst) normalized RNA-Seq data. PCA plots were generated using R package ggbiplot version 0.55.

TE Subfamilies Expression Quantification in RNA-Seq Datasets

Quantification of transposable elements was performed at the level of class (n=8), superfamily (n=112) and subfamily (n=1180) as annotated in UCSC RepeatMasker on human genome (hg38). Reads were intersected with the UCSC Repeat Masker annotation using intersectBed from BedTools 2.29.2 (with “-split” parameter) and reads with a minimum of 10 bp overlap with the repeat in strand-specific manner were used for counting. To mitigate the effect of multi-mapping reads on the counts, reads overlapping multiple repeat loci of same subfamily were counted one time, and the same was done at superfamily and class level. Additionally, read pairs were counted as single unit. This allowed for unbiased counting for long and short repeat regions. Normalized read counts were calculated using DESeq2 on all class, superfamily and subfamily.

De Novo Reconstruction of Novel LINE1 Containing Transcripts

A comprehensive catalogue of LINE1 containing transcripts in quiescent naïve CD4⁺ T-cells chromatin compartment was generated by combining two different approaches for de novo transcripts assembly. Briefly, chromatin RNA-seq reads mapped in proper pairs (sam flags 99, 147, 83 and 163) from 4 biological replicates were pooled together, amounting to a total of 113 million properly read pairs. To reconstruct transcripts containing TEs with a greater confidence, two independent algorithms were used: Trinity 2.8.4 75 in genome-guided mode (--SS_lib type FR --genome_guided_bam --genome_guided_max_intron 10000 --genome_guided_min_reads_per_partition 3) in tandem with PASA 2.3.3⁷⁶ (-C -R --AL T_SPLICE --ALIGNERS blat,gmap --CPU 1 --transcribed_is_aligned_orient), and StringTie 2.0 7⁷ (--rf -a 3). Mono-exonic transcripts were removed from further analysis as already done in 78,79 to filter out possible artefactual transcripts due to transcriptional noise or low polymerase fidelity, furthermore they are difficult to be assessed bioinformatically and need extensive manual curation. Multi-exonic transcripts intersecting with TEs (UCSC Repeatmasker) were selected. In order to obtain a new and consistent catalogue of non-redundant transcripts, only those transcripts sharing the TE-containing exon (intersectBed -f 0.8 -r -s) identified by both the assemblers were selected. A unified set of TE transcripts was obtained by merging the selected transcripts using StringTie (merge -1-f 0). TE transcripts were annotated using gffcompare 0.11.2 against transcripts from GENCODE version 25 GTF file. Finally, de novo reconstructed TE transcripts having at least 20 bp of overlap between an exon and a LINE1 locus were annotated as LINE1 containing transcripts, retrieving 3072 transcripts. Genes containing LINE1 transcripts within their genomic position are henceforth referred as “LINE1 containing genes”.

Expression Quantification of Novel LINE1 Transcripts

We estimated the expression of LINE1 transcripts using Salmon 1.1.0 (in selective alignment mode with default parameters) in order to minimize the confounding effects of similar and partially overlapping canonical transcriptional isoforms and to include multi-mapping reads. The salmon index was built using a reference transcriptome comprising both the transcripts from GENCODE version 25 and the reconstructed TE transcriptome (see “De novo reconstruction of novel LINE1 containing transcripts” subheading).

Filtering of Novel LINE1 Transcripts

Firstly, we filtered LINE1 transcripts more expressed (higher TPM values) in the chromatin fraction compared to nucleoplasm fraction of naïve CD4⁺ T-cells in at least 3 out of 4 replicates and less expressed (lower TPM values) in activated T-cells compared to CD4⁺ naïve T-cells in at least 2 out of 3 replicates, retrieving 1884 LINE1 containing transcripts specifically enriched in the chromatin of naïve CD4⁺ T-cells. The 1884 LINE1 containing transcripts were assigned to an intergenic region or to a transcriptional unit (gene) by intersecting them to known transcripts from GENCODE version 32 using intersectBed from bedtools 2.29.2. If the assignment for a unique gene or intergenic region was not possible, the gene was classified as “ambiguous”. We found that 1647 out of 1884 transcripts annotate to known transcriptional units, 81 were intergenic and 156 were ambiguous (see also “LINE1 transcripts characterization” subheading). Of the 1647 transcripts intersecting a transcriptional unit, 112 were assigned to non-coding genes and 1535 to protein coding genes, of which 1469 were in the same orientation (for LINE1 transcripts intersecting with both coding and non-coding GENCODE transcriptional units, the protein coding transcripts were used for the assignment). 1013 transcripts out of the 1469 transcripts have a LINE1 containing exon at the beginning (one of the first two exons) or at the end (one of the last two exons) of the novel LINE1 transcripts. Furthermore, among the 1013 LINE1 containing transcripts we selected those having a LINE1 containing exon with an average H3K36me3 signal two times the average H3K9me3 signal, as an evidence of transcription of that chromatin region 80,81 H3K36me3 and H3K9me3 signals were obtained by processing Roadmap Epigenomics pre-aligned ChIP-seq data (see ChIP-seq data analysis). Finally, we discarded LINE1 transcripts whose LINE1 exons elongate and overlap with host gene's UTR, as possible artefacts of already annotated transcripts. This strategy led to the identification of 461 novel LINE1 transcripts.

LINE1 Transcripts Characterization

LINE1 subfamily enrichment analysis of the newly identified 461 LINE1 transcripts was performed using Fisher's exact test against the genomic distribution of all 132 LINE1 subfamilies present in human genome (hg38) as annotated in UCSC RepeatMasker (Refer to Extended data FIG. 4 c,d). LINE1 loci were annotated for LINE1 features (5′UTR, ORF1, intergenic, ORF2 and 3′UTR) based on their alignment on L1.4 (GenBank accession L19092.1). L1.4 sequence was annotated using LIXplorer, LINE1 loci were aligned against L1.4 using blastn. In order to capture all LINE1 loci, which can be evolutionary similar or divergent to the L1.4 sequence, blastn was performed with two different parameters: one for closely related sequences (-word_size 4 -gapopen 5 -gapextend 2 -reward 2 -penalty -3 -dust no -soft_masking false) and another for divergent sequences (-word_size 4 -gapopen 8 -gapextend 6 -reward 5 -penalty -4 -dust no -soft_masking false). Blast hits with highest coverage of LINE1 loci were selected as the best hits and were annotated for LINE1 features based on their position of the alignment on L1.4 sequence (refer to Extended data FIG. 4f). LINE1 transcripts with PAS detected in at least three of the five replicates were considered as polyadenylated (refer to Extended data FIG. 4g). Splice motifs consensus sequences were searched in the 461 LINE1 transcripts. Sequence logo representation of the consensus sequence at the boundaries of the LINE1-containing exons at the start or end of the LINE1 transcripts were generated using WebLogo 3.7.4 (refer to Extended data FIG. 4h).

LINE1 Transcripts Validation by PCR

LINE1 transcripts were validated by PCR with GoTaq G2 Flexi DNA polymerase (Promega, cat. num. M7806). PCR reactions were performed on naïve CD4⁺ T-cells cDNA (RT minus was used to verify the splicing of the novel transcriptional variants). Primers were designed on IFNGR2.L1, MED23.L1, HIRA.L1, EED.L1, ASH2L.L1, ARCP2.L1, DDX6.L1, RAB22a.L1 transcripts and on the corresponding canonical mRNAs. PCR amplicons were controlled by electrophoresis on 1.6% agarose gel. All the transcripts have been validated in at least 3 different individuals.

Gene Expression Quantification and Controls in RNA-Seq Datasets

Aligned reads were used to generate read counts per gene using HTSeq v0.12.4 (htseq-count -s yes --nonunique all) against GENCODE version 25 and normalized to fragments per kilobase per million (FPKM) using as library size the total number of reads mapping within the coordinates of gene models. Expression values of LINE1 containing genes were selected among all quantified genes.

Ingenuity Pathway Analysis

The 407 LINE1 containing genes were included in network analyses performed using Ingenuity Pathway Analysis (Ingenuity® Systems, www.ingenuity.com). The list of the LINE1 containing gene identifiers was uploaded into in the application. Each gene identifier was mapped to its corresponding gene object in the Ingenuity Pathways Knowledge Base and is called focus gene. For the generation of the Networks, 70 genes per network and a significance score>40 were considered. Networks are represented in relation to the subcellular localization of the genes and the connectivity is based on direct (continuous lines) and indirect relationships (dashed lines). In the graphical representation, genes are represented as nodes and the biological relationship between two nodes is represented as an edge.

Nanopore cDNA Library Preparation and Sequencing

Libraries were prepared starting from 75 ng of chromatin RNA, using the PCR-cDNA Barcoding Kit (Oxford Nanopore Technologies, UK) and following manufacturer's guidelines. RNA from three independent samples were individually processed and barcoded, and the final libraries were pooled together for the sequencing run. Libraries quality and average size was checked by TapeStation (Agilent, Calif., USA). Sequencing was performed using a MinION platform and the R9.4.1 flowcell (Oxford Nanopore Technologies).

Nanopore Sequencing Data Analysis

Nanopore cDNA signal were processed into demultiplexed reads using Guppy basecalling software version 5.0.7 with parameters “guppy_basecaller --flowcell FLO-MIN106 --kit SQK-PCB109 --barcode_kits SQK-PCB109 -trim_barcodes”. Reads from three biological replicates were aligned on the reference transcriptome containing Gencode v25 and reconstructed TE-containing transcripts (see “De novo reconstruction of novel LINE1 containing transcripts” subheadeing) using minimap2 version 2.17-r941 with parameter “-ax map-ont”. The presence of LINE1 transcripts in the Nanopore data were tested by selecting transcripts that were uniquely aligned. The transcriptional profile of LINE1 transcripts using long reads was obtained by calculating the coverage using bedtools genomecov with parameters “-ignoreD -bg”, dividing LINE1 transcripts into 100 bins and calculating the mean and standard error of the mean per bin using R 3.6.2.

Chromatin Immunoprecipitation (ChIP)

ChIP assay was performed as described in⁸² with minor modifications. Quiescent and 16 h activated naïve CD4⁺ T-cells were cross-linked in 1% formaldehyde. Crosslinked cells were lysed in sonication buffer (10 mM TrisHCl pH 8, 2 mM EDTA, 0.25% SDS, supplemented with 1× complete EDTA-free protease inhibitor (Roche) and 1 mM PMSF (Merck)). Chromatin was sheared (BRANSON A250 with a 3.2-mm tapered microtip; five cycles of 1 min at 25% amplitude, 50% of duty cycle), checked on 0.9% agarose gel run at 70V. Immunoprecipitation was performed by incubating 25 μg of chromatin diluted in 1.5× equilibration buffer (10 mM TrisHCl pH 8, 233 mM NaCl, 0.166% Na-Deoxycholate, 1.66% Triton X-100, 1 mM EDTA, 1× complete EDTA-free protease inhibitor and 1 mM PMSF) with 1-2 μg of antibodies (H3K4me3 (Millipore, cat. num. 07-473) and H3K36me3 (Abcam, cat.num. Ab9050)), overnight, on a rotating wheel at 4° C. The immunocomplexes were recovered with magnetic Dynabeads (protein G; Invitrogen) for 2 h on the wheel at 4° C. The beads were washed two times with 600 μL RIPA Low Salt (10 mM TrisHCl pH8, 100 mM NaCl, 1 mM EDTA, 0.1% SDS, 0.1% Na-Deoxycholate, 1% Triton X-100), two times with 600 μL RIPA High Salt (10 mM TrisHCl pH8, 500 mM NaCl, 1 mM EDTA, 0.1% SDS, 0.1% Na-Deoxycholate, 1% Triton X-100), two times with 600 μL RIPA-LiCl (10 mM TrisHCl pH8, 250 mM LiCl, 1 mM EDTA, 0.5% Na-Deoxycholate, 0.5% NP-40) and finally with 600 μL of 10 mM TrisHCl pH8. All RIPA buffers were complemented with 1× complete EDTA-free protease inhibitor and 1 mM PMSF. Crosslinking reversion was performed by incubating the beads in Elution buffer (10 mM TrisHCl pH8, 2% SDS) at 65° C. overnight. Immunoprecipitated DNA was purified with AMPure XP PCR Purification beads (Beckman Coulter) following manufacturer's instruction. DNA libraries were prepared with 25 ng of purified DNA using the NEBNext Ultra I DNA Library Prep Kit for Illumina (NEB), without size selection and 8 PCR amplification cycles. The libraries were purified using AMPure XP beads, eluted in Nuclease free H₂O, and sequenced 150 pb paired on a NextSeq 500 platform (Illumina).

ChIP-Seq Data Analysis

For the LINE1 transcripts selection, ENCODE published datasets in CD4⁺ naïve T-cells for H3K36me3 (ENCFF152WXT, ENCFF3240ZH, ENCFF416GLM, ENCFF783JQO) and H3K9me3 (ENCFF197EDP, ENCFF287UWA, ENCFF338SVK, ENCFF753UAT), with their relative input samples (ENCFF044KMD, ENCFF343ILJ, ENCFF421BMD, ENCFF737YRO) were used. Fold enrichment of the ChIP was performed using macs 2.2.6, relative to its control input with non-default parameters “-f BAM -g 3049315783 -p le-2 --nomodel --extsize [average fragment size provided by ENCODE]--keep-dup all -B --SPMR --broad” for the callpeak module, and “-m FE” for the bdgcmp module. The enrichment signal of H3K36me3 compared to H3K9me3 was calculated as log 2 ratio using deeptools 3.4.1 bigWigCompare with 10 bp as bin size.

Further, H3K36me3 and H3K4me3 ChIP-seq were generated to inspect the chromatin of LINE1 containing genes in T-cell activation or LINE1 knock down (see above). Reads from technical replicates were pooled together and reads quality before and after trimming were assessed using FastQC 0.11.9. Reads were trimmed for low quality base calls using Trimmomatic 0.39 in paired-end mode with parameters: “ILLUMINACLIP:TruSeq3-PE.fa:2:30:10 LEADING:3 TRAILING:3 SLIDINGWINDOW:4:15 MINLEN:50”, or in single-end mode with adapters reference file “TruSeq3-SE.fa” and same parameters as above. Trimmed reads were aligned to the human genome assembly hg38 using Bowtie 1.2.3 with parameters “-m 1 --best --strata -v 3” and “-X 2000 --fr” for paired-end only. After alignment, paired-end reads not mapped in proper pair, as well as duplicated reads, were removed using Samtools 1.9 83. ChIP peaks were called using macs 2.2.6 callpeak module giving as input the alignment file of the ChIP target and its relative control input, with parameters “--keep-dup all -g 3049315783 -B -p 0.01” for both H3K4me3 and H3K36me3 samples and “--broad” for H3K36me3 only, paired-end specific paramterers “-f BAMPE” and single-end specific paramters “-f BAM --nomodel --extsize 200”. The coverage tracks were calculated by subtracting the background signal from the fragment pileup using macs2 bdgcmp module with parameters “-m FE”. As control for the LINE1 containing genes, a set of the same number of genes was randomly sampled from a pool of protein coding genes marked by at least one H3K36me3 peak using the GNU coreutils built-in “shuf” command. The positional distribution of H3K36me3 and H3K4me3 on LINE1 containing genes and control genes was obtained by dividing gene models into 40 bins, while -1.5 and +3 kb flanking regions were smoothed with 150 bp long bins using deeptools 3.4.1 computeMatrix with parameters “-m 6000 -b 3000 -a 3000 -bs 150”, the average between replicates and the median between genes was calculated and a cubic smoothing spline was fitted into the data using R 3.6.2 built-in “smooth.spline” function.

Motif Enrichment Analysis

Analysis of motif enrichment were performed using the AME algorithm from the MEME suite version 5.3.3 with parameters “--scoring avg --method fisher --hit-lo-fraction 0.25 --evalue-report-threshold 10.0 --control --shuffle-- --kmer 2” for both DNA and RNA binding motifs. Transcription factors binding motifs provided by the HOCOMOCO Human (v11 CORE) database were searched on putative promoter sequences of LINE1 transcripts host genes. Promoter regions were obtained by intersecting H3K4me3 and H3K27Ac peaks from two biological replicates found within 5 kb upstream and 1 kb downstream of host genes transcription starting sites using bedtools version 2.29.2. TFs with a log 2 fold change CD8⁺/CD4⁺<−2 in proteomics analysis and in <0 in RNA-seq datasets were considered as “Top ranking CD4⁺ specific TFs”. RNA binding proteins motifs provided by Ray et al. 2013 (PMID23846655) were searched on the LINE1 RNA sequences contained in the LINE1 exon of the novel transcripts.

Results

Inventors herein provide observations regarding the dynamics of LINE1 expression in human T lymphocytes from peripheral blood, in dysfunctional/anergic T cells in vitro and in TILs, and their identification in CD4+ naïve T cells.

LINE1 RNAs is Expressed at Chromatin in CD4+ Naïve T Cells and Regulated by mTORC1 Pathway Upon Activation and Differentiation.

To investigate the expression of TEs in human T-lymphocytes, we probed LINE1, Alu and HERV superfamilies with RNA FISH and qRT-PCR in quiescent naïve and memory CD4⁺ and CD8⁺ T-cells, isolated from healthy individuals. We observed that LINE1 RNAs are specifically expressed in the nuclei of quiescent naïve CD4⁺ T-cells (FIG. 1a-c). Alu RNAs rather show a broad perinuclear distribution in all T-cell subsets (FIG. 9a-c), while HERV RNAs are poorly expressed (FIG. 9d-f). LINE1 RNAs are almost exclusively present in the chromatin fraction of naïve CD4+ T-cells (FIG. 1d and FIG. 9g) and are associated with open chromatin regions as determined by colocalization with H3K4me3 histone mark (FIG. 9h,i). Prolonged actinomycin D treatments moderately affect LINE1 RNAs levels, indicating that these RNAs are not transcribed at high rate in naïve CD4+ T-cells (FIG. 1e). We then analyzed which is the LINE1 RNAs dynamics in T-cell activation and differentiation, finding that they are rapidly downregulated and remain at low level during differentiation towards effector cells (i.e. Th1, Th2 Th17, FIG. 1f and FIG. 9j,k).

We hypothesized that LINE1 RNAs levels are finely regulated by T-cell specific signaling pathways. Thus, we treated activated or differentiated CD4⁺ T-cells with different immunosuppressive drugs that target mTORC1, Calcineurin, or NF-cB pathways (FIG. 1g), finding that the mTORC1 inhibitor Rapamycin restores LINE1 RNAs levels in activated and differentiated T-cells (FIG. 1h and FIG. 9l-n). To strengthen these in vitro data, we asked whether LINE1 RNAs levels were affected by mTORC1 inhibition in vivo. We thus investigated LINE1 expression in memory CD4⁺ T-cells isolated from blood of kidney transplanted patients treated with the mTORC1 inhibitor Everolimus, and of Lymphangioleiomyomatosis (LAM) [MIM: 606690] patients 20 treated for life with the Rapamycin analog Sirolimus²¹. Consistent with the inhibitory role of mTORC1 on LINE1 RNAs expression in vitro, we found that, at variance with healthy individuals, memory CD4⁺ T-cells of these patients re-express LINE1 RNAs (FIG. 1i-k). Therefore, we demonstrate that LINE1 RNAs are enfolded at chromatin in CD4+ T cells where they are rapidly downregulated following T-cell activation in a mTORC1 dependent fashion.

The LINE1 Expressed in Naïve CD4+ T Cells are Spliced in Non-Canonical Transcript Variants of Cell Activation Genes.

In order to determine which LINE1 elements are expressed and how LINE1 transcripts are constituted, we sequenced chromatin and nucleoplasm RNA from quiescent naïve CD4⁺ T-cells. As initial analysis, we counted reads of TE classes, superfamilies and families and then compared read counts in chromatin and nucleoplasmic fractions. We confirmed that among TE classes, LINE, and in particular L1M family (evolutionary old, present in primates and widely in other mammals) is the most expressed and chromatin enriched, whereas the families L1P and L1H are expressed at low level and enriched in nucleoplasm (FIG. 10a,b). This result is different with what found in mESCs, where the evolutionary young, retrotransposition competent, L1md_T and L1md_A subfamilies are more expressed in respect to the evolutionary old L1_Mus1 and L1_Mus3 (¹¹ and FIG. 10a,b). In T-cells, almost the half of LINE1 reads are chimeric (i.e. mapping both on LINE1 and on a non-repeated region) and 79% of reads derive from LINE1 localized in protein coding genes (FIG. 10d,e), thus most likely included in a novel transcript variant. In mESCs, the majority of LINE1 reads are entirely deriving from LINE1 elements with a more broad genomic distribution (FIG. 10f,g), supporting that different LINE1 are expressed in mouse development in respect to human T-cells. In order to identify the transcript variants containing LINE1, we applied a de novo stranded genome-guided transcriptome assembly using two algorithms, Trinity²² and StringTie²³ (see methods). We identified 3072 multi-exonic transcripts containing at least one exon with LINE1. To obtain a reliable list of LINE1 transcripts we have applied several filtering criteria based on consistency of their presence among different individuals and evidence of LINE1 exon transcription at chromatin level (H3K36me3/H3K9me3 ratio^{24,25}) We retrieved 461 LINE1 transcripts that are non-canonical spicing variants originated from 407 protein coding genes. The presence of 88% of these LINE1 transcripts could be validated and accurately reconstructed with long nanopore reads performed on the chromatin fraction of naïve CD4⁺ T-cells (FIG. 11a) and several were confirmed by rt-PCR in naïve CD4⁺ T-cells isolated from 3 different individuals (FIG. 11b-i). The spliced LINE1 are short in length (371 bp on average), mainly ORF2 truncated elements and enriched in distinct L1M subfamilies (i.e. L1ME4a, L1MC4, L1ME4b) (FIG. 12a-c); in particular, 80% of these LINE1 are located within an intron (FIG. 12d) and spliced as novel exons that contain LINE1 and an intronic fragment (FIG. 12e). Of note, these are evolutionary old LINE1 elements, that represent truncated form of full length LINE1 elements that have been remodeled during evolution ending in degenerated elements that are unable to retrotranspose and do not codify for any of the protein required for retrotrasposition. Indeed, to demonstrate that the observed mechanism is independent from the retrotransposition mechanism, we treated naïve CD4+ T-cells with 3TC inhibitors and we performed RNA-FISH finding that the staining of LINE1 RNAs was completely unchanged (FIG. 12f).

FIG. 2a show as to how representative LINE1 transcripts (e.g. ARPC2) are reconstructed. HIRA.L1 presence in naïve CD4+ T-cell and their downregulation upon activation was further confirmed with single molecule RNA-FISH (smRNA-FISH), detecting the unique portion of the LINE1 exon (FIG. 2b,c and FIG. 13a).

In order to reckon the functional relevance of the 407 protein coding genes from which LINE1 transcripts derive, we used IPA (Ingenuity Pathway Analysis) networks analysis and found a direct and consistent relationship with genes required for cellular activation (e.g. gene expression, cell signaling and cell to cell interactions, cell cycle). Together, the above experiments identify a large set of previously unknown non-canonical transcripts variants of genes required for cellular activation, so suggesting these transcripts are involved in maintaining CD4+ T-cell quiescence.

Since LINE1 transcripts derive from cell-activation genes and are localized at chromatin, we asked whether they could regulate expression of the corresponding protein coding genes (hereafter named canonical transcripts). First, we observed that LINE1 transcripts are localized in cis at their genomic loci as exemplified in combo DNA-RNA FISH experiments for HIRA and RABB22A (FIG. 2d and FIG. 13c). Then, we depleted naïve CD4+ T-cells of HIRA.L1 or RABB22A.L1 transcripts with antisense oligonucleotides (ASOs) designed on LINE1 exon non repeated region and found that only the corresponding canonical transcript was upregulated (FIG. 2 e,f and FIG. 13d). Finally, we deleted the LINE1 from the intron of IFNGR2 and ARPC2 genes using Cas9 RNP complexes with sgRNAs on unique regions flanking the repeat and proved that in naïve CD4+ T-cells i) LINE1 element is necessary to originate LINE1 transcript and ii) LINE1 transcripts regulatory role is strictly in cis (FIG. 2g-i and FIG. 13e-h). Therefore, our results suggest that LINE1 transcripts keep paused at chromatin expression of the corresponding canonical transcripts.

LINE1 Transcripts Act in Complex with Nucleolin Reducing the Expression of the LINE1 Containing Genes in Cis in Naïve CD4+ T-Cells.

We asked how LINE1 transcripts epigenetically control the expression of genes from which they originate. We knocked down LINE1 transcripts by treating quiescent naïve CD4⁺ T-cells with LINE1 ASOs for 48 h (FIG. 3a,b). Interestingly, we observed that upregulation of LINE1 containing genes occurs in quiescent naïve CD4⁺ T-cells that are knocked down for LINE1 RNAs (FIG. 3c). By reason of LINE1 RNAs can regulate chromatin condensation and gene silencing^{38,49,51}, we asked whether knock down of LINE1 transcripts affected chromatin organization in quiescent T-cells. We thus assessed the level of several histone marks (i.e., H3K36me3, H3K4me3, H3K9me3, H3K27me3) by quantitative western blot of histone extracts⁷³ and immunostaining in naïve T-cells treated for 48 hours with LINE1 ASOs, and found that LINE1 RNAs depletion results in a marked increase of H3K36me3, so indicating chromatin remodeling towards active transcription, which occurs in the absence of cell activation (FIG. 3d and FIG. 14a-c). Interestingly, we performed H3K36me3 ChIP-seq of naïve T-cells depleted of LINE1 RNAs and found that the H3K36me3 increase is specific for LINE1 containing genes, the same is observed upon T-cell activation (FIG. 3e and FIG. 14 d-h), so demonstrating that the LINE1 containing genes can be freed by the LINE1 transcripts repressive activity in the absence of activation.

Since it has been reported in mESCs that Nucleolin, a LINE1 RNAs binding protein⁸⁶, is complexed with LINE1 RNAs and Kap1 to regulate cell identity and 2C stage differentiation genes⁵¹, we asked what was the relation between Nucleolin and LINE1 RNAs in T-cells. We performed RNA immune precipitation for Nucleolin finding that it is in complex with LINE1 transcripts (FIG. 3h). Interestingly, Nucleolin ASOs treatment phenocopies the effect of LINE1 ASOs in promoting the transcription of the LINE1 containing genes and the increase of H3K36me3 (FIG. 15 a,d). When we depleted Nucleolin in quiescent naïve CD4⁺ T-cells by treating them with specific ASOs for 48 hours, LINE1 RNAs levels remained unchanged, but after subcellular fractionation we observed that LINE1 RNAs association to chromatin was reduced, indicating that Nucleolin is involved in LINE1 RNAs chromatin compartmentalization. (FIG. 3i,j and FIG. 15 e). Finally, we asked whether LINE1 RNAs modulation could affect T-cell effector function. First, naïve CD4+ T-cells depleted for LINE1 RNAs were activated and differentiated for 7 days to Th1 effector cells, observing that these cells doubled the production of transcription factor Tbet and the secretion of effector cytokine IFNγ (FIG. 3k,l); importantly the same phenotype was observed for Nucleolin knock down in the same condition (FIG. 3m,n). All together, these results indicate that LINE1 RNAs modulate the transcriptional switch from quiescence to activation state in naïve CD4⁺ T-lymphocytes, acting at chromatin in complex with Nucleolin via H3K36me3 chromatin remodeling.

LINE1 Transcripts are Regulated by IRF4 Transcription Factor in CD4+ T-Cells.

As LINE1 transcripts derive from genes involved in cell activation, we asked the reason why CD8+ T-cells do not express LINE1 transcripts unlike CD4+ T-cells, which are developmentally close. Therefore, we profiled the expression of the 461 LINE1 transcripts and the canonical transcripts using RNA seq data of T-cells progenitors and naïve and activated CD4+ and CD8+ T-cells (FIG. 4a, see methods). Among all T-cell precursors, LINE1 transcripts are uniquely expressed by naïve CD4+, corroborating previous results (FIG. 4b and FIG. 1a-c). Interestingly, canonical transcripts are CD4+ T-cell specific as well: in particular, in activated CD4+ T-cells LINE1 transcripts are downregulated whereas canonical transcripts are upregulated (FIG. 4b,c and Extended data FIG. 16a,b). We then searched for transcription factors (TF) more expressed in CD4+ than CD8+ T-cells in RNA-seq (see methods) and proteomics data sets²⁶ and whose motifs are enriched in LINE1 containing gene promoters, that could account for the differential regulation of these loci. With this analysis we top ranked IRF4, that we found interesting because it is a key factor reported in CD4+ T-cell activation^{27,28} (FIG. 16c). First, we verified that IRF4 is almost absent in CD8+ T-cells (FIG. 4d) and upregulated in CD4+ T-cell activation (FIG. 16d) and second, we demonstrated by ChIP that IRF4 is bound at LINE1 containing genes promoters in naïve CD4+ and not CD8+ T-cells (FIG. 4e); then, we depleted IRF4 in naïve CD4+ T-cells using IRF4 ASOs (FIG. 4f and FIG. 16e,f) and observed that both LINE1 and canonical transcripts are strongly downregulated (FIG. 4g), indicating that this TF controls their CD4+ specific expression. Overall, these data suggest that during T-cell development, LINE1 and the corresponding canonical transcripts are CD4+ T-cell specific under the control of IRF4 transcription factor.

Upon T-Cell Activation, LINE1 Transcripts are Downregulated by Splicing Repressors PTBP1/MATR3 and Elongating Factor GTF2F1, Favoring Canonical Transcripts Expression.

Since IRF4 is a key factor in T-cell activation and is directly involved in LINE1 containing gene loci regulation, in what way are LINE1 transcripts downregulated in activated CD4+ T-cells?Several heteromeric RNA binding proteins have been already reported by Attig et al. to bind intronic LINE1 influencing their lineage-specific splicing; in particular PTBP1 and MATR3 suppress RNA splicing within and around LINEs²⁹. Moreover, since we reported that LINE1 RNAs downregulation is under the control of mTORC1 (FIG. 1), we crossed this dataset with that of Hsu et al.⁺that thoroughly described the proteins regulated by mTORC1. We identified only one protein, GTF2F1, that binds intronic LINE1 and that is also regulated by mTORC1. GTF2F1 is a transcription elongation factor that is phosphorylated upon T cell activation³¹. Thus, we have investigated the role of PTBP1, MATR3 and GTF2F1 in the regulation of LINE1 transcripts in activated CD4+ T-cells. We performed RNA immunoprecipitation experiments with PTBP1 and GTF2F1 demonstrating that these two factors bind LINE1 exons specifically in activated CD4+ T-cells (FIG. 5a-d). In details, as exemplified for RABB22A, PTBP1 is binding only the pre-mRNA, in line with its splicing suppressive role, whereas GTF2F1 is binding both the pre-mRNA and the spliced canonical transcript, as expected for an transcriptional elongating factor (FIG. 5e-g). Indeed, when we depleted PTBP1, MATR3 and GTF2F1 with ASOs in naïve CD4+ T-cells and then activated them, we found that LINE1 transcripts are more expressed while canonical transcripts are less (FIG. 5h and FIG. 17), so indicating a causal inverse relation between LINE1 and canonical transcripts. We deduce that LINE1 transcripts are non-canonical splicing variants that are suppressed by PTBP1/MATR3/GTF2F1 to favor the expression of canonical transcripts in cell activation.

LINE1 Transcripts Re-Accumulate in TILs Via IRF4 and Nucleolin and by the Loss of PTPB1/GTF2F1 Mediated Mechanism.

We analyzed LINE1 RNAs dynamics in T-cells isolated from tumor microenvironments where effector T-cells are often rendered dysfunctional. Although recent reports have described the contribution of transcription factors and epigenetic modifications to the dysfunctional state, the underlying mechanisms are not well-defined⁹¹-93 We thus evaluated LINE1 RNAs content in CD4⁺ and CD8⁺ T-cells isolated from several Colorectal Cancers (CRC), Non-Small-Cell Lung Cancers (NSCLC), and the corresponding non-tumoral adjacent tissues. Notably, we observed LINE1 RNAs signal in intratumoral memory CD4⁺ T-cells and, surprisingly, also in intratumoral memory CD8⁺ T-cells of all tumor samples, while in the non-tumoral adjacent tissues, similarly to what is observed in peripheral blood, no signal was detected in memory T-cells (FIG. 6a,b). Then, we explored LINE1 RNAs expression in vitro in exhausted-dysfunctional CD4+ and CD8+ T cells by exposing CD4+ and CD8+ T cells every 2 days to stimulatory anti-CD3 mAb^{66 67} Repetitive anti-CD3 stimulation induces an expected growth arrest, PD-1 upregulation and reduction of effector cytokines secretion, both in CD4⁺ and CD8⁺ T cells (FIG. 18). Interestingly, we observed a consistent re-accumulation of LINE1 RNAs in the nuclei of these dysfunctional and CD4⁺CD8⁺ T-cells: more in detailed we observed a specific increase of LINE1 transcripts with a concomitant decrease of LINE1 containing genes expression (FIG. 6c-f). We found that exhausted CD4+ and CD8+ T cells, in line with LINE1 transcripts accumulation, have higher amount of the transcription factors IRF4 protein level and Nucleolin, while reduce the abundance of GTF2F1 in exhausted CD4+ and CD8+ T-cells (FIG. 7a). In particular, by mean of RIP assay we discovered that LINE1 transcripts are more bound to Nucleolin while they lost PTBP1 and GTF2F1 binding (FIG. 7b). IRF4 knock down ascertain that this transcription factor is responsible for LINE1 transcription also in exhausted context (FIG. 7c,d and FIG. 18e). Overall these set of data demonstrate that in exhausted T-cells and in TILs LINE1 transcripts re-accumulate, and this is due by IRF4—nucleolin mediated LINE1 transcripts generation and stabilization at chromatin, while the suppressive mediated mechanism through PTBP1/GTF2F1 is lost.

LINE1 Transcripts Level Controls the Effector Response of TILs.

In order to assess whether the dysfunctional behavior observed in intratumoral T-cells could be at least in part ascribed to LINE1 RNAs accumulation and thus be modulated by LINE1 targeting, we isolated intratumoral CD4⁺ and CD8⁺ T-cells and knocked down their LINE1 transcripts with LINE1 ASOs. Cells depleted for LINE1 were then measured for Inhibitor Checkpoint expression, effector cytokines production and target cell killing ability (FIG. 8a). We found that PD-1, LAG3 and TIM3 percentage of positive cells was reduced upon LINE1 targeting (FIG. 8b,c) and in line with these results we observed also an increase of effector cytokines secretion (IFNγ and Granzyme B for CD4⁺ and CD8⁺ T cells, PerforinA for CD8⁺ T cells) in memory TILs depleted for LINE1 (FIG. 8d,e). These data suggest an increased functionality of TILs and to corroborate this, we also measured target cell killing abilities of memory CD4⁺ and CD8⁺ TILs treated with LINE1 ASO compared to the intratumoral T-cells treated with irrelevant ASOs, verifying that killing ability of TILs is almost doubled upon LINE1 knock down (FIG. 8f,g).

As a corollary to this, LINE1 RNAs knockdown in T-cells that were previously rendered exhausted in vitro results restore effector cytokines secretion (IFNγ and Granzyme B for CD4⁺ and CD8⁺ T cells, PerforinA for CD8⁺ T cells) (FIG. 19a-e) and of killing abilities (FIG. 19f,g) while proliferation is not modified by LINE1 ASOs treatment (FIG. 19h,i). Collectively, our findings demonstrate that LINE1 RNAs levels regulate T-cell effector response and their accumulation in intratumoral T-cells associates to a dysfunctional behavior that could be partially reversed with LINE1 RNAs ASOs.

Sequences

Consensus sequence of LINE1 sequences that are mostly represented in the LINE1 transcripts reconstructed in T-cells.

>LIME4A L1
Homo sapiens
(SEQ ID NO: 1)
cttgtatccagaatatataaagaacgcctacaact
caacaataaaaaaacgaatttcccaacaaaaaaac
ggacaaaggacacgaanagaccgtttacaaaagaa
gaaatggaaataactancgaacatgaaaaatgttc
aacctcactaataatcaaagaaatgcaaattaaaa
caacaatgagatnccgttcttcntcgtctancaaa
ctggcanagatataaaaagataatakccagtgttg
gtgaggatgtggagaaacgggcactctcatacact
gctggtgggagtataaattggtacaacctttctgg
aaggcaatttggcaatatntatcaaaagccttaaa
aatgttcataccctttgacccagcaattccacttc
taggaatctatcctaaggaaataatcagaaatgtg
nacaaagatttacgtacaaagatgttcaccgcagt
attatttataatagcaaaaaattggaaacaaccta
aatgtccaataataggggantggttaaataaatta
tggtacatccatacaatggaatattatgcagccat
taaaaatnatgttttcgaagaatatttaatgacat
gggaaaatgctcatgatataatgttaagtgaaaaa
agcaggntacaaaactgtatatacagtatgatctc
aactttgttataaaattacatatataaatgtatac
gtatntacatagaaaaaagactggaaggaaataca
ccaaaatgttaacagtggttatctctgggtggtgg
gattatgggtgatttttattttcttttttctttgt
attttctgtattttccaaattttctacaatgaaca
tgtattacttttataatcagaaaaaaaa
>L1M4B L1
Homo sapiens
(SEQ ID NO: 2)
aaggagtttcacttctggaatggcagcatgaggag
ctccgnagacccnctccccagcgaaacaancataa
ctggtgaaaattatttttaaaaaaacaaccattta
aagtctctggaaattgtcctaagggcatacagcaa
atgaagaaacatttattcaagaaaatctactaaat
ctcagtaagaacagtgagagtctgtggcacttgag
ccacgacccgctcccaccctcccccctccccagct
cagcntgacagaagctccactccgggcgggtgcgg
ccaagaagacggggctccctctcccctcagctccc
agtcaagggntacggtatctcnccgggaggggcag
gccgccagcatttctcatcccctccagctccgngt
tgcagaggctaaattccaggtgagtgtagctgaga
ggtcgggggctcccttcctccacccagcccccact
catagggoggaggctctaccccaggcgcggcaggc
cgagaatactggggccctgattgccctcaccccag
ctcgctcatagggcggaggttccacgccgggagag
gcaagccgagaagaccagaggctaccgcccccgcc
cagcgccctgctcataaagcaggggtgtcactccg
agagaagcgggccactgtccccgcccccagctccg
gagcagtggctcagagattttgcccagggggagag
gcagnccataagaacagagagctccgaagctctcc
ccaaaggaactgactttatttgaaacagagtgtgg
ggaagttcaagcctaagggtactctcgaaaacaat
ggagattttggtggtaagcaattaagaggaggctg
gtagctccatgagagcaacaagctaaaccataggc
cagctagtttaccagagagaaccagggaaagagac
agctaagaagagccctcctggggtcagaacaaacc
tcaaagactggcctcaaaaactacccctrcaaagg
ggcccgaatttaattggatcagactgtggagcaat
ttatgccccagggcattgtcgaaaacaatagagca
atcagccggcaattagtggagcctaacagctgggt
gtgataccaanngaggcagacagettaacagagag
atcagggaaagagacagtcaaagagagccctgcta
aaaccactgtcatcccagggtgactgtgcgcatgc
ccaaggctgcgccctctgaggagcgacatcagagg
cttcacactgngggggaaatagacttcactaaaat
agtccagccaagtcactaaacaaataaacaagcaa
aaacaancacnangagccgggggnggggaatcagt
atccagagttgctacaatatattacctaaaatgtc
cagttttcaacaaaaaattatgagacatgcaaaga
aacaggaaagtgtgacccatacacaggaaaaaaag
caggcaacagaaactgcctgtgagagggcccagat
gtcggatttagcagacaaagacttcaaagcagcca
ttataaatatgttcaaagaactaaaggaaaccatg
cttaaagaagtaaaggaaggtatgatgacaatgtc
tcatcaaatagagantatcaataaagagatagaaa
ttataanaaaaaaccaaatggaaattctggagttg
aaaagtacaataactgaaatgaaaaattcactaga
ggggctcaacagtagatttganctggcagaagaaa
agaatcagtraacttgaagatagatcaatagagat
tatgcaatctgaagaacagaaagaaaaaaaagaat
gaagaaaaatgaacagagcctcagagaaatgtggg
acaccatyaagcataccaacatatacatacatgga
cagacaaacaacatatacataatgggagtaccaga
aggagaggagaagagagagaaaggagcagaaaaaa
tatttgaagaaataatggctaaaaacttcccaaat
ttgatgaaaaacattaatattaatctacacatcca
agaagctcaataaactccaagtaggataaactcaa
agagatccacacctagacacatcatagtcaaaatg
ttgaaagacaaagacaaagagaaaatcttgaaagc
agcaagagaaaaatgactcatcacatacaagggaa
nnnacctcaataagattaacagctgacttctcatc
agaaacaatggaggccagaaggcagtgggatgaca
tattcaaagtgctgaaagaaaaaaaaaaaaaaaaa
aaaacaaaaaaahacanaaacaaatacaacnytac
ctgtcaaccaagaattctatatccagcaaaactat
ctttcaaaaatgaaggtgaaataaagacattccca
gataaacaaaaactgagagaatttgttgctagcag
acctaccttacaagaaatactaaaggaagagttct
tcaggctgaaaggcaagtgacaccagatagtaatt
caaatccacataaaaaaataaagagacacacacta
agtaaagnncactagtaaaggtaattatgtagnaa
gacagtaanttaattatnaaagrcatgtakgtaat
tataaaagacagtataaatgcatatttcttctttc
ttctcttaactgatttaaaaagcaattgtataaaa
caatatgtatataattgtattgttgggcctataac
atatagaaatgtaatatatttgacaataacagcac
aaaggaggtgggtgggagcaaagctgtattggagt
aaggaaatgacaccagatggtaacttgaatccaca
ggaacaaatgaagagaaccagaaatggtaaataag
aaggttaatataacaaactctataaatatatactt
gttctcctttcttctcttctttaaaagacataaaa
ttatataaagtaataattataacaaatgtatttnt
nnnataataatgttgggtttgtaacatatatagat
gtatatatattnntattgtaatatgtataacaata
atagcacaaaaaaggagaaaaaggaatagagctat
ataggagtaacatttctatatctcactggaattaa
gttagtataaatctgaagtagattctgataangtt
aagatgtatatggtaagccctagagcaaccactaa
gaaaataacttaaaaaaatatagtaaaaaaaatca
ttaaagaaattaaaatgttacactagaaaatattc
acttaatgcaaaagaaagcagtaaaggaggaatag
aggaacaaaaaagacatgagacatatnacatatag
aaaacaaaaagtaaaatggcagatataaatccaac
tatatcaatataacattaaatgtgattatggatta
aryaaaatggcaraagctgtcagnctngagattta
ntntatataaatccaantnnntngttnanatgntn
agacngntaatncaaatatcaataataacattaaa
tgtgaatggattaaacaatccaatcaaaaggcaga
gattgtcagactggataaaaaaaaaaaaacaagat
ccaactatatgctgtctacaggagacacactttag
attcaaagatacaaatagrttgaaagtaaaaggat
ggaaaaagatatatcatgcaaacagcaaccataag
aaagctggagtggctatactaatatcagacaaaat
agactttaaaacaaaaaatgttactagagataaag
agggacattttattatataatgataaaagggtcaa
aagggtcaatccatcaggaagatataacaattata
aacatatatgcatatanatatatgcacctaacaac
agagcccccaaaatacatgaagcaaaaactgacag
aaatgaagggagaaatagacaattcaacaataata
gttggagacttcaataycccactttcaataatgga
tagaacaactaggcagaagnnaatangatcaacaa
ggaaatagaagacttgaacaacactataaaccaac
tagacctaacagacatctatagaacatttatagaa
cactcyatccaacaacagcagaatatacattcttc
tcaagtgcacatggaacattctccaggatagacca
tatgctaggccataaaacaagyctcaataaattta
tttaaaggattgaaataatacaaagtatgttctct
gaccacaatggaatgaaattagaaatcaataacaa
aaaatttgggaaatttacaaatatgtggaaattaa
acaacacactcctaaataaccaatgggtcaaagaa
gaaatcacaagagaaattagaaaatactttgagat
gaatgaaaatgaagacacaacataccaaaatttat
gggatgcagctaaagcagtgyttagaggaaaattt
atagctgtaaatgcctatattaaaaaagaagaaag
atctcaaatcaataacctaaccttctaccttaaga
cactaaaaaaagaagagcaaactaaacctaaagca
agcagaaggaaggaaataataaagattagagcaga
aattaatgaaatagaagaaaaacaatagagaaaat
caatgaaaccaaaagctggttctttgaaaagatca
acaaaattgacaaacctttagctagactgaccaag
aaaaagagaagactcaaattactaaaatcagaaat
gaaagagggaacattactactaaccttacagaaat
aaaaaggattataaaggaatactatgaacaattgt
atgccaataaattnagataacttagatgaaatgga
caaattcctagaaanyaagacacacaaactacyaa
aactgactcaagaagaaataganaatctgaataga
cctataaaantnaagagattgaattagtaatntaa
aaactnccyacaaaaaaagcccagncccagatggc
ttcactggtgaattctccaaanatttaaaanagaa
ttaataccaattattcacctnttccaaaaaataga
agaggaggnaayactnccnaactnattctatgagg
ccagtattatcctgataccaaaaccagncaaagac
atnacaaaagaaaagaaaa
>L1MC4 L1
Homo sapiens
(SEQ ID NO: 3)
ctaatatacctaatatacaaaaaactcttaaaatt
gaaggataaaaagncaaaaaccnaatannaaaatg
ggnaaaagacatgaacagacaattcacnaaaaatn
ataaaatggcccttaagcatataaaaagatgttca
ncctcacntataattagagaaacgcaaattaaaac
tacaccgagataccatttctcacccancagategg
caaaaattaaaaagtatggcaatatannctgttgg
cgaggctgtggggnaacnggnactctcatacactg
ctggtgggagtgcaaattggtacaactnctttgga
aganaatttggcagtntctaataaaactacacntg
cntttacactttgacccattagtcccacttctaga
aatttaccctanagaaatacttctaacagntcaaa
aatacacatgtacagggatgttcatagcagtntta
ttnntaatngtaaaanattggaaacaatcnaaatg
tccatcagcaggagaatggntgaataaactatggt
ncatccacacaatggaatactatncagctgtaaaa
aagaatgaggaagatctctgtaataatgtggagng
atttcggaacatnntnttnagttgaaaaagcnang
cgcaaaagagtatatatantatgctacccttcata
taagaaagaaggggatatgagaaaatatacatata
tctgctcatttgtgcaaaaagaaacacagaaaaga
taaancaganactaatgagattggttacccacagg
gaannggtgggaatggggaggaaaggacggaagga
atggggggcagtgacacttttctgagtataccttt
ttgtatagttctaacttttgnaaccatgttaatgt
ttcacatactcaagaaatgaataantaaaatcaac
aaggatggggganaactcaaaatgaaatacaaaca
gaaacaaatgaaccwaactgtatttcaaatgaata
acataaccacactgaagggggtnaggaagaaaaga
actaacccaagtaacttttgaacacagtattttga
ctatatgccctcaggctaaagacaaaaagaactnt
aaacaaatattgaactctagttagtaggcttattt
tccgcagnggcatgggttagcaattctgaaactac
tttctgtatattctaggactgagcaaataagtaaa
tatattgnggataatgggagccaggtttctcactg
tcggagaagggagttacaaatatggaaagggggaa
gactagaatgaaccctgtggtgttggattggaatt
ggaggtatcagtgtgaactcatggtttttaatata
natagatatacagacagacagatatagaaatagat
atagatatatatgtgtntgtgtatatgtgtatgta
tatacgtacatatatttcctagctctgtccactga
gagggcctagaagcaatgacaccccagtagcaatg
agcacacctagcgcccagatcttggtttctaaata
ccattctccactaaaaggaaccagggctccttgga
gaaatggctgattccagggctggggcagggaaagt
acaagatgagcctggaacatcttgttgtgccagaa
agtaaggaagtgctcaaagaatgatggggacatgt
caaaaggacacaggagccagcttgaaggggctccc
actggccaaatctgggacaatttgagcatcaaaat
aaataatgatagtaatggattataacccattgaat
aaaataagaatccatgagtccatactgatataaat
aaataaataaataaatgggggagaagggaaagctc
ttccttacagtagaatgccaactaataaatgtaga
aggaatgatggaattagaaaatcaccatttggcaa
ccatcatagtaataattgattcaggcaagaatcat
caatggatgctaaaactagtgggtgaaagtttgat
gagnaacaggatatttacatagtctcaaagtatct
ccccacaaaatacttattaattacaaaggggaaaa
tagtaactttacagtggagaaacctggcagacacc
accttaaccaagtgatcaaagttaacatcaccagt
aatgggacaaatcgacatcatgtgcctcctgatat
gatgcactgagaaggacacaacatcacttctgtgg
tattcctgccaaaaatgcataacctgaatctaatc
atgaggaaacatcagacaaacccaaattgagggac
attctacaaaataactggcctgtactcttcaaaaa
tgtcaaggtcatgaaagacaaagaaagactgagga
actgttccagattaaaggagactaaagagacatga
caactaaatgcaacgcgtgatcctggattggatcc
tggaccaganttttttttgctataaaggacattat
tgggacaactggcgaaatttgaataaggtctgtag
attagataatagtattgtatcaatgttaatttcct
gattttgatnattgtactgtggttatgtaagagaa
tgtccttgtttttaggaaatacacactgaagtatt
taggggtaanggggcatcatgtctgcaacttactc
tcaaatggttcagaaaaaaaaatatgtatatgnan
acagagaatgataaagcaaatgtggcaaaatgtta
acatttggggaatctgggtgaagggtatacgggaa
ttctttgtactattcttgcaacttttctgtaagtc
tgaaattatttcaaaataaaaagttaaaaaa

LINE1 ASOs are able to target these LINE1 elements, that are those specifically expressed in naïve CD4+ T-cells (see results).

The nomenclature for the above sequences is as follows (below the IUPAC nucleotide code and the corresponding Base):

A: Adenine

C: Cytosine

G: Guanine

T (or U): Thymine (or Uracil)

R: A or G

Y: C or T

S: G or C

W: A or T

K: G or T

M: A or C

B: C or G or T

D: A or G or T

H: A or C or T

V: A or C or G

N: any base

.or-:gap

### REFERENCES

1 de Koning, A. P., Gu, W., Castoe, T. A., Batzer, M. A. & Pollock, D. D. Repetitive elements may comprise over two-thirds of the human genome. PLoS Genet 7, e1002384, doi:10.1371/joural.pgen.1002384 (2011).
2 Lander, E. S. et al. Initial sequencing and analysis of the human genome. Nature 409, 860-921, doi:10.1038/35057062 (2001).
3 Chuong, E. B., Elde, N. C. & Feschotte, C. Regulatory activities of transposable elements: from conflicts to benefits. Nat Rev Genet 18, 71-86, doi:10.1038/nrg.2016.139 (2017).
4 Feschotte, C. Transposable elements and the evolution of regulatory networks. Nat Rev Genet 9, 397-405, doi:10.1038/nrg2337 (2008).
Percharde, M., Sultana, T. & Ramalho-Santos, M. What Doesn't Kill You Makes You Stronger: Transposons as Dual Players in Chromatin Regulation and Genomic Variation. Bioessays 42, e1900232, doi:10.1002/bies.201900232 (2020).
6 Kazazian, H. H., Jr. & Moran, J. V. The impact of L1 retrotransposons on the human genome. Nat Genet 19, 19-24, doi:10.1038/ng0598-19 (1998).
7 Viollet, S., Monot, C. & Cristofari, G. L1 retrotransposition: The snap-velcro model and its consequences. Mob Genet Elements 4, e28907, doi:10.4161/mge.28907 (2014).
8 Okada, N., Hamada, M., Ogiwara, I. & Ohshima, K. SINEs and LINEs share common 3′ sequences: a review. Gene 205, 229-243, doi:10.1016/s0378-1119(97)00409-5 (1997).
9 Malik, H. S., Henikoff, S. & Eickbush, T. H. Poised for contagion: evolutionary origins of the infectious abilities of invertebrate retroviruses. Genome Res 10, 1307-1318, doi:10.1101/gr.145000 (2000).
Canapa, A., Barucca, M., Biscotti, M. A., Forconi, M. & Olmo, E. Transposons, Genome Size, and Evolutionary Insights in Animals. Cytogenet Genome Res 147, 217-239, doi:10.1159/000444429 (2015).
11 Kazazian, H. H., Jr. Mobile elements: drivers of genome evolution. Science 303, 1626-1632, doi:10.1126/science.1089670 (2004).
12 Belancio, V. P., Hedges, D. J. & Deininger, P. Mammalian non-LTR retrotransposons: for better or worse, in sickness and in health. Genome Res 18, 343-358, doi:10.1101/gr.5558208 (2008).
13 Muotri, A. R. et al. Somatic mosaicism in neuronal precursor cells mediated by L1 retrotransposition. Nature 435, 903-910, doi:10.1038/nature03663 (2005).
14 Beck, C. R. et al. LINE-1 retrotransposition activity in human genomes. Cell 141, 1159-1170, doi:10.1016/j.cell.2010.05.021 (2010).
Ewing, A. D. & Kazazian, H. H., Jr. High-throughput sequencing reveals extensive variation in human-specific L1 content in individual human genomes. Genome Res 20, 1262-1270, doi:10.1101/gr.106419.110 (2010).
16 Iskow, R. C. et al. Natural mutagenesis of human genomes by endogenous retrotransposons. Cell 141, 1253-1261, doi:10.1016/j.cell.2010.05.020 (2010).
17 Baillie, J. K. et al. Somatic retrotransposition alters the genetic landscape of the human brain. Nature 479, 534-537, doi:10.1038/nature10531 (2011).
18 Perrat, P. N. et al. Transposition-driven genomic heterogeneity in the Drosophila brain. Science 340, 91-95, doi:10.1126/science.1231965 (2013).
19 Upton, K. R. et al. Ubiquitous L1 mosaicism in hippocampal neurons. Cell 161, 228-239, 50 doi:10.1016/j.cell.2015.03.026 (2015).
20 Coufal, N. G. et al. L1 retrotransposition in human neural progenitor cells. Nature 460, 1127-1131, doi:10.1038/nature08248 (2009).
21 Reilly, M. T., Faulkner, G. J., Dubnau, J., Ponomarev, I. & Gage, F. H. The role of transposable elements in health and diseases of the central nervous system. J Neurosci 33, 17577-17586, 55 doi:10.1523/JNEUROSCL3369-13.2013 (2013).
22 Saleh, A., Macia, A. & Muotri, A. R. Transposable Elements, Inflammation, and Neurological Disease. Front Neurol 10, 894, doi:10.3389/fneur.2019.00894 (2019).
23 Payer, L. M. & Burns, K. H. Transposable elements in human genetic disease. Nat Rev Genet 20, 760-772, doi:10.1038/s41576-019-0165-8 (2019).
24 Deniz, O., Frost, J. M. & Branco, M. R. Regulation of transposable elements by DNA modifications. Nat Rev Genet 20, 417-431, doi:10.1038/s41576-019-0106-6 (2019).
25 Mc, C. B. The origin and behavior of mutable loci in maize. Proc Natl Acad Sci USA 36, 344-355, doi:10.1073/pnas.36.6.344 (1950).
26 McClintock, B. Controlling elements and the gene. Cold Spring Harb Symp Quant Biol 21, 197-216, doi:10.1101/sqb.1956.021.01.017 (1956).
27 Sundaram, V. et al. Widespread contribution of transposable elements to the innovation of gene regulatory networks. Genome Res 24, 1963-1976, doi:10.1101/gr.168872.113 (2014).
28 Bodega, B. & Orlando, V. Repetitive elements dynamics in cell identity programming, maintenance and disease. Curr Opin Cell Biol 31, 67-73, doi:10.1016/j.ceb.2014.09.002 (2014).
29 Pennisi, E. Genomics. ENCODE project writes eulogy for junk DNA. Science 337, 1159, 1161, doi:10.1126/science.337.6099.1159 (2012).
30 Bourque, G. et al. Evolution of the mammalian transcription factor binding repertoire via transposable elements. Genome Res 18, 1752-1762, doi:10.1101/gr.080663.108 (2008).
31 Imbeault, M., Helleboid, P. Y. & Trono, D. KRAB zinc-finger proteins contribute to the evolution of gene regulatory networks. Nature 543, 550-554, doi:10.1038/nature21683 (2017).
32 Morgan, H. D., Sutherland, H. G., Martin, D. I. & Whitelaw, E. Epigenetic inheritance at the agouti locus in the mouse. Nat Genet 23, 314-318, doi:10.1038/15490 (1999).
33 Ferrari, R. et al. TFIIIC Binding to Alu Elements Controls Gene Expression via Chromatin Looping and Histone Acetylation. Mol Cell 77, 475-487 e411, doi:10.1016/j.molcel.2019.10.020 (2020).
34 Schmidt, D. et al. Waves of retrotransposon expansion remodel genome organization and CTCF binding in multiple mammalian lineages. Cell 148, 335-348, doi:10.1016/j.cell.2011.11.058 (2012).
35 Zhang, Y. et al. Transcriptionally active HERV-H retrotransposons demarcate topologically associating domains in human pluripotent stem cells. Nat Genet 51, 1380-1388, doi:10.1038/s41588-019-0479-7 (2019).
36 Faulkner, G. J. et al. The regulated retrotransposon transcriptome of mammalian cells. Nat Genet 41, 563-571, doi:10.1038/ng.368 (2009).
37 Rodriguez-Terrones, D. et al. A distinct metabolic state arises during the emergence of 2-cell-like cells. EMBO Rep 21, e48354, doi:10.15252/embr.201948354 (2020).
38 Lu, J. Y. et al. Genomic Repeats Categorize Genes with Distinct Functions for Orchestrated Regulation. Cell Rep 30, 3296-3311 e3295, doi:10.1016/j.celrep.2020.02.048 (2020).
39 Attig, J. et al. Heteromeric RNP Assembly at LINEs Controls Lineage-Specific RNA Processing. Cell 174, 1067-1081 e1017, doi:10.1016/j.cell.2018.07.001 (2018).
40 Nekrutenko, A. & Li, W. H. Transposable elements are found in a large number of human protein-coding genes. Trends Genet 17, 619-621, doi:10.1016/s0168-9525(01)02445-3 (2001).
41 Perepelitsa-Belancio, V. & Deininger, P. RNA truncation by premature polyadenylation attenuates human mobile element activity. Nat Genet 35, 363-366, doi:10.1038/ng1269 (2003).
42 Roy-Engel, A. M. et al. Human retroelements may introduce intragenic polyadenylation signals. Cytogenet Genome Res 110, 365-371, doi:10.1159/000084968 (2005).
43 Gong, C. & Maquat, L. E. lncRNAs transactivate STAU1-mediated mRNA decay by duplexing with 3′ UTRs via Alu elements. Nature 470, 284-288, doi:10.1038/nature09701 (2011).
44 Kapusta, A. et al. Transposable elements are major contributors to the origin, diversification, and regulation of vertebrate long noncoding RNAs. PLoS Genet 9, e1003470, doi:10.1371/joural.pgen.1003470 (2013).
45 Kelley, D. & Rinn, J. Transposable elements reveal a stem cell-specific class of long noncoding RNAs. Genome Biol 13, R107, doi:10.1186/gb-2012-13-11-r107 (2012).
46 Fort, A. et al. Deep transcriptome profiling of mammalian stem cells supports a regulatory role for retrotransposons in pluripotency maintenance. Nat Genet 46, 558-566, doi:10.1038/ng.2965 (2014).
47 Lu, X. et al. The retrovirus HERVH is a long noncoding RNA required for human embryonic stem cell identity. Nat Struct Mol Biol 21, 423-425, doi:10.1038/nsmb.2799 (2014).
48 Hall, L. L. et al. Stable COT-1 repeat RNA is abundant and is associated with euchromatic interphase chromosomes. Cell 156, 907-919, doi:10.1016/j.cell.2014.01.042 (2014).
49 Jachowicz, J. W. et al. LINE-1 activation after fertilization regulates global chromatin accessibility in the early mouse embryo. Nat Genet 49, 1502-1510, doi:10.1038/ng.3945 (2017).
50 Fadloun, A. et al. Chromatin signatures and retrotransposon profiling in mouse embryos reveal regulation of LINE-1 by RNA. Nat Struct Mol Biol 20, 332-338, doi:10.1038/nsmb.2495 (2013).
51 Percharde, M. et al. A LINE1-Nucleolin Partnership Regulates Early Development and ESC Identity. Cell 60 174, 391-405 e319, doi:10.1016/j.cell.2018.05.043 (2018).
52 Quezada, S. A. & Peggs, K. S. Tumor-reactive CD4+ T cells: plasticity beyond helper and regulatory activities. Immunotherapy 3, 915-917, doi:10.2217/imt.11.83 (2011).
53 Catalano, I., Grassi, E., Bertotti, A. & Trusolino, L. Immunogenomics of Colorectal Tumors: Facts and Hypotheses on an Evolving Saga. Trends Cancer 5, 779-788, doi:10.1016/j.trecan.2019.10.006 (2019).
54 Jerby-Arnon, L. et al. A Cancer Cell Program Promotes T Cell Exclusion and Resistance to Checkpoint Blockade. Cell 175, 984-997 e924, doi:10.1016/j.cell.2018.09.006 (2018).
55 Jamal-Hanjani, M., Thanopoulou, E., Peggs, K. S., Quezada, S. A. & Swanton, C. Tumour heterogeneity and immune-modulation. Curr Opin Pharmacol 13, 497-503, doi:10.1016/j.coph.2013.04.006 (2013).
56 Munn, D. H. & Bronte, V. Immune suppressive mechanisms in the tumor microenvironment. Curr Opin Immunol 39, 1-6, doi:10.1016/j.coi.2015.10.009 (2016).
57 Yang, Y. Cancer immunotherapy: harnessing the immune system to battle cancer. J Clin Invest 125, 3335-3337, doi:10.1172/JC183871 (2015).
58 Galon, J. & Bruni, D. Approaches to treat immune hot, altered and cold tumours with combination immunotherapies. Nat Rev Drug Discov 18, 197-218, doi:10.1038/s41573-018-0007-y (2019).
59 Guo, X. et al. Global characterization of T cells in non-small-cell lung cancer by single-cell sequencing. Nat Med 24, 978-985, doi:10.1038/s41591-018-0045-3 (2018).
60 Lavin, Y. et al. Innate Immune Landscape in Early Lung Adenocarcinoma by Paired Single-Cell Analyses. Cell 169, 750-765 e717, doi:10.1016/j.cell.2017.04.014 (2017).
61 Lei Zhang, X. Y., Liangtao Zheng, Yuanyuan Zhang, Yansen Li, Qiao Fang, Ranran Gao, Boxi Kang, Qiming Zhang, Julie Y. Huang, Hiroyasu Konno, Xinyi Guo, Yingjiang Ye, Songyuan Gao, Shan Wang, Xueda Hu, Xianwen Ren, Zhanlong Shen, Wenjun Ouyang & Zemin Zhang. Lineage tracking reveals dynamic relationships of t cells in colorectal cancer. Nature, doi:10.1038/s41586-018-0694-x (2018). (2018).
62 Savas, P. et al. Single-cell profiling of breast cancer T cells reveals a tissue-resident memory subset associated with improved prognosis. Nat Med 24, 986-993, doi:10.1038/s41591-018-0078-7 (2018).
63 Bray, F. et al. Global cancer statistics 2018: GLOBOCAN estimates of incidence and mortality worldwide for 36 cancers in 185 countries. CA Cancer J Clin 68, 394-424, doi:10.3322/caac.21492 (2018).
64 Travis, W. D. Pathology of lung cancer. Clin Chest Med 23, 65-81, viii, doi:10.1016/s0272-5231(03)00061-3 (2002).
65 Siegel, R. L., Miller, K. D. & Jemal, A. Cancer statistics, 2018. CA Cancer J Clin 68, 7-30, doi:10.3322/caac.21442 (2018).
66 Dunsford L. S., T. R. H., Rathbone E., Patakas A. A Human In Vitro T Cell Exhaustion Model for Assessing Immuno-Oncology Therapies. (Springer, 2020).
67 Yang, Z. Z. et al. TGF-beta upregulates CD70 expression and induces exhaustion of effector memory T cells in B-cell non-Hodgkin's lymphoma. Leukemia 28, 1872-1884, doi:10.1038/leu.2014.84 (2014).
68 Ricciardi, S. et al. The Translational Machinery of Human CD4(+) T Cells Is Poised for Activation and Controls the Switch from Quiescence to Metabolic Remodeling. Cell Metab 28, 961, doi:10.1016/j.cmet.2018.09.010 (2018).
69 Marasca, F., Cortesi, A. & Bodega, B. 3D COMBO chrRNA-DNA-ImmunoFISH. Methods Mol Biol 2157, 281-297, doi:10.1007/978-1-0716-0664-3_16 (2021).
70 Shibayama, Y., Fanucchi, S. & Mhlanga, M. M. Visualization of Enhancer-Derived Noncoding RNA. Methods Mol Biol 1468, 19-32, doi:10.1007/978-1-4939-4035-6_3 (2017).
71 Cortesi, A. et al. 4q-D4Z4 chromatin architecture regulates the transcription of muscle atrophic genes in facioscapulohumeral muscular dystrophy. Genome Res 29, 883-895, doi:10.1101/gr.233288.117 (2019).
72 Fontana, C. et al. Early maternal care restores LINE-1 methylation and enhances neurodevelopment in preterm infants. BMC Med 19, 42, doi:10.1186/s12916-020-01896-0 (2021).
73 Bodega, B. et al. A cytosolic Ezhl isoform modulates a PRC2-Ezhl epigenetic adaptive response in postmitotic cells. Nat Struct Mol Biol 24, 444-452, doi:10.1038/nsmb.3392 (2017).
74 Werner, M. S. & Ruthenburg, A. J. Nuclear Fractionation Reveals Thousands of Chromatin-Tethered Noncoding RNAs Adjacent to Active Genes. Cell Rep 12, 1089-1098, doi:10.1016/j.celrep.2015.07.033 (2015).
75 Grabherr, M. G. et al. Full-length transcriptome assembly from RNA-Seq data without a reference genome. Nat Biotechnol 29, 644-652, doi:10.1038/nbt.1883 (2011).
76 Haas, B. J. et al. Improving the Arabidopsis genome annotation using maximal transcript alignment assemblies. Nucleic Acids Res 31, 5654-5666, doi:10.1093/nar/gkg770 (2003).
77 Pertea, M. et al. StringTie enables improved reconstruction of a transcriptome from RNA-seq reads. Nat Biotechnol 33, 290-295, doi:10.1038/nbt.3122 (2015).
78 Ramsay, L. et al. Conserved expression of transposon-derived non-coding transcripts in primate stem cells. BMC Genomics 18, 214, doi:10.1186/s12864-017-3568-y (2017).
79 Ranzani, V. et al. The long intergenic noncoding RNA landscape of human lymphocytes highlights the regulation of T cell differentiation by linc-MAF-4. Nat Immunol 16, 318-325, doi:10.1038/ni.3093 (2015).
80 Kolasinska-Zwierz, P. et al. Differential chromatin marking of introns and expressed exons by H3K36me3. Nat Genet 41, 376-381, doi:10.1038/ng.322 (2009).
81 Spies, N., Nielsen, C. B., Padgett, R. A. & Burge, C. B. Biased chromatin signatures around polyadenylation sites and exons. Mol Cell 36, 245-254, doi:10.1016/j.molcel.2009.10.008 (2009).
82 Schmidl, C., Rendeiro, A. F., Sheffield, N. C. & Bock, C. ChIPmentation: fast, robust, low-input ChIP-seq for histones and transcription factors. Nat Methods 12, 963-965, doi:10.1038/nmeth.3542 (2015).
83 Li, H. et al. The Sequence Alignment/Map format and SAMtools. Bioinformatics 25, 2078-2079, doi:10.1093/bioinformatics/btp352 (2009).
84 Han, J. S., Szak, S. T. & Boeke, J. D. Transcriptional disruption by the L1 retrotransposon and implications for mammalian transcriptomes. Nature 429, 268-274, doi:10.1038/nature02536 (2004).
85 Ustyugova, S. V., Lebedev, Y. B. & Sverdlov, E. D. Long L1 insertions in human gene introns specifically reduce the content of corresponding primary transcripts. Genetica 128, 261-272, doi:10.1007/s10709-005-5967-2 (2006).
86 Peddigari, S., Li, P. W., Rabe, J. L. & Martin, S. L. hnRNPL and nucleolin bind LINE-1 RNA and function as host factors to modulate retrotransposition. Nucleic Acids Res 41, 575-585, doi:10.1093/nar/gks1075 (2013).
87 Hsu, P. P. et al. The mTOR-regulated phosphoproteome reveals a mechanism of mTORC1-mediated inhibition of growth factor signaling. Science 332, 1317-1322, doi:10.1126/science.1199498 (2011).
88 Tan, H. et al. Integrative Proteomics and Phosphoproteomics Profiling Reveals Dynamic Signaling Networks and Bioenergetics Pathways Underlying T Cell Activation. Immunity 46, 488-503, doi:10.1016/j.immuni.2017.02.010 (2017).
89 Moir, L. M. Lymphangioleiomyomatosis: Current understanding and potential treatments. Pharmacol Ther 158, 114-124, doi:10.1016/j.pharmthera.2015.12.008 (2016).
90 Sehgal, S. N. Sirolimus: its discovery, biological properties, and mechanism of action. Transplant Proc 35, 7S-14S, doi:10.1016/s0041-1345(03)00211-2 (2003).
91 Martinez, G. J. et al. The transcription factor NFAT promotes exhaustion of activated CD8(+) T cells. Immunity 42, 265-278, doi:10.1016/j.immuni.2015.01.006 (2015).
92 Philip, M. et al. Chromatin states define tumour-specific T cell dysfunction and reprogramming. Nature 545, 452-456, doi:10.1038/nature22367 (2017).
93 Yue, X., Lio, C. J., Samaniego-Castruita, D., Li, X. & Rao, A. Loss of TET2 and TET3 in regulatory T cells unleashes effector function. Nat Commun 10, 2011, doi:10.1038/s41467-019-09541-y (2019).

1. An isolated human T cell, B cell, NK cell or Tumor cell, or a composition comprising said cell, wherein said cell is stably or transiently affected in the expression of (long interspersed element 1) LINE1 (L1), wherein L1 comprises a sequence having 100, 99, 98, 97, 96, 95, 90, 85, or 80% identity with SEQ ID NO: 1 or 2 or 3.

2. (canceled)

3. (canceled)

4. A method for treating a primary or secondary immunodeficiency, or a pathology that displays an immunosuppressed phenotype, comprising at least one step of administering to an individual in need a suppressor or inhibitor of (long interspersed element 1) LINE1 (L1) expression or a cell as defined in claim 1, wherein LI comprises a sequence having 100, 99, 98, 97, 96, 95, 90, 85, or 80% identity with SEQ ID NO: 1 or 2 or 3, wherein the suppressor or inhibitor is at least one molecule selected from the group consisting of:
a) a polynucleotide, selected from the group consisting of: antisense construct, antisense oligonucleotide, RNA interference construct or siRNA or a polynucleotide coding for it,
b) a vector comprising or expressing the polynucleotide as defined in a),
c) a CRISPR/Cas9 component,
d) a host cell genetically engineered expressing a polypeptide or antibody or comprising the polynucleotide as defined in a) or at least one component of c.

5. The method according to claim 4, wherein the polynucleotide is an isolated inhibitory nucleic acid targeting LINE1.

6. The method according to claim 5, wherein the inhibitory nucleic acid comprises a sequence of nucleotides that are complementary to 10 to 50 consecutive nucleotides of SEQ ID NO: 1 or 2 or 3.

7. The method according to claim 5, wherein said inhibitory nucleic acid is at least one RNA inhibitor from the group consisting of: antisense oligo (ASO), gapmer, mixmer, shRNA, siRNA, stRNA, and snRNA.

8. The method according to claim 7, wherein the ASO comprises a sequence capable of hybridizing or complementary to a sequence comprising: SEQ ID NO: 1 or 2 or 3.

9. (canceled)

10. The method according to claim 4, wherein the cell or the inhibitor or suppressor of LINE1 is administered in combination with an immunotherapy or with a radiotherapy or chemotherapeutic agent or with targeted therapies which promote raising of new antigens and immunity response or with immunity system adjuvants.

11. The method according to claim 4, being performed in Adoptive cell transfer (ACT), cell therapy treatment, mismatched bone marrow transplantation, mismatched NK cell infusion or cytokine-induced killer (CIK) cell infusion, or wherein said suppressor or inhibitor or the cell is injected in the tumour site, or delivered by nanoparticles specifically to the site of interest.

12. (canceled)

13. A method to modulate the commitment of naive CD4+ T naive cells towards any effector lineage and to modulate the effector response in dysfunctional T cells comprising the step of inhibiting LINE1 expression in said cells, wherein the step of inhibiting LINE1 expression in said cells is performed by means of at least one suppressor or inhibitor of L1, wherein L1 comprises a sequence having 100, 99, 98, 97, 96, 95, 90, 85, or 80% identity with SEQ ID NO: 1 or 2 or 3, wherein the suppressor or inhibitor is at least one molecule selected from the group consisting of:
a) a polynucleotide, selected from the group consisting of: antisense construct, antisense oligonucleotide, RNA interference construct or siRNA or a polynucleotide coding for it,
b) a vector comprising or expressing the polynucleotide as defined in a),
c) a CRISPR/Cas9 component, and
d) a host cell genetically engineered expressing said polypeptide or comprising the polynucleotide as defined in a) or at least one component of c).

14. (canceled)

15. (canceled)

16. (canceled)

17. The isolated cell according to claim 1, wherein said cell is a CD4+ T naive cell or a CD8+ T cell, or a dysfunctional T cell.

18. The isolated cell according to claim 17, wherein said dysfunctional T cell is a Tumor Infiltrating Lymphocyte (TIL).

19. The method according to claim 4, wherein the method is performed by immunotherapy.

20. The method according to claim 4, wherein the pathology that displays an immunosuppressed phenotype is a cancer or a metastasis.

21. The method according to claim 20, wherein the cancer is lung cancer or colorectal cancer (CRC).

22. The method according to claim 7, wherein said inhibitory nucleic acid is a 2′-deoxy-2′-fluoro-D-arabinonucleid acid (FANA) ASO, and comprises one or more modified bonds or bases, or said inhibitory nucleic acid comprises one or more modified bonds or bases.

23. The method according to claim 5, wherein said inhibitory nucleic acid is at least one RNA inhibitor which is an antisense oligo (ASO) comprising a nucleic acid sequence that targets one of the following sequences or the corresponding RNA sequence:
LINE1-b
(SEQ ID NO: 5)
GGACCTCTTCAAGGAGAACTA
LINE1-c
(SEQ ID NO: 6)
GAAGTTGAATCTCTGAATAGA
LINE1-d
(SEQ ID NO: 7)
GGACCTCTTCAAGGAGAACTA
LINE1-e
(SEQ ID NO: 8)
GGAGAGGATGCGGAGAAATAG,
and wherein the CRISPR/Cas9 component is a sgRNA comprising a nucleic acid sequence that targets or is complementary to one of the following sequences:
IFNGR2-F
(SEQ ID NO: 9)
ACTGATCGTGAGAGGCTTCGTGG
IFNGR2-R
(SEQ ID NO: 10)
GGTCATTTAGGGTGACAGGCAGG
ARCP2-F
(SEQ ID NO: 11)
GCTGTCATGGGAATCACGAAGGG
ARCP2-R
(SEQ ID NO: 12)
AAGGAAGACCACTTTTAAGGAGG
or to the corresponding RNA sequence.

24. The method according to claim 10, wherein said immunotherapy comprises administration of an immune checkpoint inhibitor, chimeric antigen receptor (CAR)-expressing immune effector cells, or both, wherein said immune checkpoint inhibitor is an or comprises one or more anti-CD137 antibodies; anti-PD-1 (programmed cell death 1) antibodies; anti-PDLI (programmed cell death ligand 1) antibodies; anti-PDL2 antibodies; or anti-CTLA-4 antibodies.

25. A method for treating a viral disease, comprising at least one step of administering to an individual in need a suppressor or inhibitor of (long interspersed element 1) LINE1 (L1) expression or a cell as defined in claim 1, wherein L1 comprises a sequence having 100, 99, 98, 97, 96, 95, 90, 85, or 80% identity with SEQ ID NO: 2, or wherein L1 comprises a sequence having 100, 99, 98, 97, 96, 95, or 90% identity with SEQ ID NO: i or 3, wherein the suppressor or inhibitor is at least one molecule selected from the group consisting of:
a) a polynucleotide, selected from the group consisting of: antisense construct, antisense oligonucleotide, RNA interference construct or siRNA or a polynucleotide coding for it,
b) a vector comprising or expressing the polynucleotide as defined in a),
c) a CRISPR/Cas9 component, and
d) a host cell genetically engineered expressing said polypeptide or comprising the polynucleotide as defined in a) or at least one component of c).

26. The method according to claim 25, wherein said viral disease is an immunodeficiency due to Human Immunodeficiency Virus (HIV) or Lymphocytic choriomeningitis virus (LCMV).

27. The method according to claim 13, wherein said polynucleotide is at least one RNA inhibitor which is an antisense oligo (ASO) comprising a nucleic acid sequence that targets one of the following sequences or the corresponding RNA sequence:
LINE1-a
(SEQ ID NO: 4)
GCACTAAATGCCTACAAGAGA.
LINE1-b
(SEQ ID NO: 5)
GGACCTCTTCAAGGAGAACTA
LINE1-c
(SEQ ID NO: 6)
GAAGTTGAATCTCTGAATAGA
LINE1-d
(SEQ ID NO: 7)
GGACCTCTTCAAGGAGAACTA
LINE1-e
(SEQ ID NO: 8)
GGAGAGGATGCGGAGAAATAG,
and wherein the CRISPR/Cas9 component is a sgRNA comprising a nucleic acid sequence that targets or is complementary to one of the following sequences:
IFNGR2-F
(SEQ ID NO: 9)
ACTGATCGTGAGAGGCTTCGTGG
IFNGR2-R
(SEQ ID NO: 10)
GGTCATTTAGGGTGACAGGCAGG
ARCP2-F
(SEQ ID NO: 11)
GCTGTCATGGGAATCACGAAGGG
ARCP2-R
(SEQ ID NO: 12)
AAGGAAGACCACTTTTAAGGAGG
or to the corresponding RNA sequence.
